(19) United States
(12) Patent Application Publication
(10) Pub. No.: US 2003/0040496 A1
(43) Pub. Date: Feb. 27, 2003

(54) Methods for halting unwanted cell growth, such as using ligand-directed nucleic acid delivery vehicles

(76) Inventors: Lois Ann Chandler, Encinitas, CA (US); Barbara A. Sosnowski, Coronado, CA (US); Andrew Baird, San Diego, CA (US); Glenn Pierce, Rancho Sante Fe, CA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 09/861,257

(22) Filed: May 17, 2001

Related U.S. Application Data
(63) Continuation of application No. 08/805,383, filed on Feb. 24, 1997, now abandoned.
Application No. 08/805,383 is a continuation-in-part of application No. 08/718,904, filed on Sep. 24, 1996.
Application No. 08/718,904 is a continuation-in-part of application No. 08/441,979, filed on May 16, 1995, now abandoned.
Application No. 08/441,979 is a continuation-in-part of application No. 08/213,446, filed on Mar. 15, 1994, now abandoned.
Application No. 08/441,979 is a continuation-in-part of application No. 08/213,447, filed on Mar. 15, 1994, now abandoned.
Application No. 08/441,979 is a continuation-in-part of application No. 08/297,961, filed on Aug. 29, 1994, now abandoned.
Application No. 08/441,979 is a continuation-in-part of application No. 08/305,771, filed on Sep. 13, 1994, now abandoned.

(51) Int. Cl. A61K 48/00; A61K 39/395

(52) U.S. Cl. 514/44; 424/143.1; 514/8

(57) ABSTRACT

Methods of treating tumors with preparations of conjugates of a receptor-binding internalized ligand and a cytocide-encoding agent are provided. The conjugates contain a polypeptide that is reactive with an FGF receptor, such as FGF2, or other ligand coupled to a nucleic acid binding domain. One or more linkers may be used in the conjugation. The linker is selected to increase the specificity, toxicity, solubility, serum stability, or intracellular availability, and promote nucleic acid condensation of the targeted moiety. The conjugates are complexed with a cytocide-encoding agent, such as DNA encoding saporin or a prodrug-encoding agent. Conjugates of a receptor-binding internalized ligand to a nucleic acid molecule are also provided.

### CROSS-REFERENCE TO RELATED APPLICATIONS

The application is a continuation-in-part of U.S. application Ser. No. 08/718,904, filed Sep. 24, 1996; which is a continuation-in-part of U.S. application Ser. No. 08/441,979, filed May 16, 1995; which is a continuation-in-parts of U.S. application Ser. No. 08/213,446, filed Mar. 15, 1994; Ser. No. 08/213,447, filed Mar. 15, 1994; Ser. No. 08/297,961, filed Aug. 29, 1994; and Ser. No. 08/305,771, filed Sep. 13, 1994.

### TECHNICAL FIELD

The present invention relates generally to the treatment of tunors, and more specifically, to the preparation and use of complexes containing receptor-binding internalized ligands, nucleic acid binding domains and cytocide-encoding agents to alter the function, gene expression, or viability of a cell in a therapeutic manner.

### BACKGROUND OF THE INVENTION

A major goal of treatment of neoplastic diseases and hyperproliferative disorders is to ablate the abnormally growing cells while leaving normal cells untouched. Various methods are under development for providing treatment, but none provide the requisite degree of specificity.

One treatment method is the administration of toxins. Immunotoxins and cytotoxins are protein conjugates of toxin molecules with either antibodies or factors which bind to receptors on target cells. The usefulness of immunotoxins and cytotoxins is limited however due to toxicity being effected prior to delivery and internalization or in non-targeted cells because of some level of non-specificity of the antibody or factor. Another limitation in the therapeutic use of immunotoxins and cytotoxins is the relatively low ratio of therapeutic to toxic dosage. Additionally, it may be difficult to direct sufficient concentrations of the toxin into the cytoplasm and intracellular compartments in which the agent can exert its desired activity.

Given these limitations, cytotoxic therapy has been attempted using viral vectors to deliver DNA encoding the toxins into cells. If eukaryotic viruses are used, such as the retroviruses currently in use, they may recombine with host DNA to produce infectious virus. Moreover, because retroviral vectors are often inactivated by the complement system, use in vivo is limited. Retroviral vectors also lack specificity in delivery; receptors for most viral vectors are present on a large fraction, if not all, cells. Thus, infection with such a viral vector will infect normal as well as abnormal cells. Because of this general infection mechanism, it is not desirable for the viral vector to directly encode a cytotoxic molecule.

While delivery of nucleic acids offers advantages over delivery of cytotoxic proteins such as reduced toxicity prior to internalization, there is a need for high specificity of delivery, which is currently unavailable with the present systems.

In view of the problems associated with gene therapy, there is a compelling need for improved treatments which are more effective and are not associated with such disadvantages. The present invention exploits the use of conjugates which have increased specificity and deliver higher amounts of nucleic acids to targeted cells, while providing other related advantages.

### SUMMARY OF THE INVENTION

The present invention generally provides methods for treating tumors. In related aspects, a pharmaceutical composition is administered to a patient, in which the composition has the formula:

receptor-binding internalized ligand—nucleic acid binding domain—cytocide-encoding agent,

receptor-binding internalized ligand—nucleic acid binding domain—prodrug-encoding agent, or

receptor-binding internalized ligand—nucleic acid binding domain—cytokine-encoding agent, wherein:

the receptor-binding internalized ligand is a polypeptide reactive with a cell surface receptor; the nucleic acid binding domain binds to a nucleic acid, the domain being chemically conjugated or fused to the receptor-binding internalized ligand; the cytocide-encoding agent, prodrug-encoding agent, or cytokine-encoding agent is a nucleic acid molecule encoding a cytocide, prodrug, or cytokine, the agent being bound to the nucleic acid binding domain; and wherein the composition binds to the cell surface receptor and is internalized.

In preferred embodiments, the receptor-binding internalized ligand is a polypeptide reactive with an FGF receptor. In other preferred embodiments, the cytocide-encoding agent encodes a protein that inhibits protein synthesis (e.g., a ribosome inactivating protein, such as saporin, gelonin, or Pseudomonas exotoxin) or that inhibits elongation factor 2, such as diphtheria toxin. In other preferred embodiments, the prodrug-encoding agent encodes HSV-thymidine kinase or cytosine deaminase. In other preferred embodiments, the cytokine-encoding agent encodes IL-2, IL-10, IL-12, or IFN-γ; or B7 and a cytokine selected from the group consisting of IL-2, IL-10, IL-12 and IFN-γ.

In yet other preferred embodiments, the nucleic acid binding domain is selected from the group consisting of helix-turn-helix motif proteins, homeodomain proteins, zinc finger motif proteins, steroid receptor proteins, leucine zipper motif proteins, helix-loop-helix motif proteins, and β-sheet motif proteins. In other preferred embodiments, the nucleic acid binding domain is a polycation, such as poly-L-lysine, poly-D-lysine, protamine, histone and spermine. Alternatively, the nucleic acid binding domain binds a DNA molecule that encodes a ribosome inactivating protein.

In preferred embodiments, the cytocide-encoding agent, prodrug-encoding agent or cytokine-encoding agent further comprises a tumor-specific promoter, such as a tyrosinase promoter, MAGE promoter, IL-2 receptor promoter, PSA-1 promoter, FGF receptor promoter, erbB2 promoter, erbB3 promoter, erbB4 promoter, MUC-1 promoter, HSP-27 promoter, CEA promoter and EGF receptor promoter, prostate specific antigen-1 promoter, probasin promoter, VEGF receptor promoter, int-1 promoter; int-2 promoter, IL-2 promoter, alpha-fetoprotein promoter, prostatic acid phosphatase promoter, prostate specific membrane antigen promoter, alpha-crystallin promoter and tie-2 promoter.

In certain embodiments, the composition further comprises a linker between the ligand and nucleic acid binding domain that increases the serum stability, intracellular availability, or condensing ability of the nucleic acid binding domain.

These and other aspects of the present invention will become evident upon reference to the following detailed description and attached drawings. In addition, various references are set forth below which describe in more detail certain procedures or compositions (e.g., plasmids, etc.), and are therefore incorporated by reference in their entirety.

### BRIEF DESCRIPTION OF THE DRAWINGS

FIG. 1 is a photograph of an SDS-PAGE of FGF2-K152 under non-reducing (left) and reducing (right) conditions. Lane 1, FGF2-K152; lane 2, FGF2; lane 3, FGF2-K152: lane 4, FGF2. The open arrow identifies material unable to enter the gel. The closed arrow identifies a protein band corresponding to FGF2.

FIG. 2 is a graph depicting the proliferation of bovine aortic endothelial cells in response to FGF2 (closed box) and FGF2-K152 (open circle) conjugate.

FIG. 3 is a photograph of a gel showing the effects of various lengths of poly-L-lysine on the ability to interact with DNA. Thirty-five ng of labeled DNA were added to increasing concentrations of either FGF2 or FGF2-K: lanes 1, 0 ng; lanes 2, 0.1 ng; lanes 3, 1 ng; lanes 4, 10 ng; lanes 5, 20 ng; lanes 6, 35 ng; lanes 7, 100 ng. Panel A: FGF2; panel B, FGF2-K152; panel C, FGF2-K13; panel D, FGF2-K84; panel E, FGF2-K267; panel F, FGF2-K39. The lengths of the digested DNA are indicated.

FIG. 4 is a chart depicting the activity of β-gal following transfection of FGF2/poly-L-lysine/DNAβ-gal into COS cells. Lane 1, 10:1 (w/w) ratio of FGF2/poly-L-lysine conjugate to DNA; lane 2, 5:1 ratio; lane 3, 2:1 ratio; lane 4, 1:1 ratio; lane 5, 0.5:1 ratio. The five bars, from left to right, are FGF2, FGF2-K13, FGF2-K39, FGF2-K84, and FGF2-K152.

FIG. 5 are photographs of toroid formation observed by electron microscopy. The upper panel shows an example of a toroid; the lower panel shows an incomplete toroid.

FIG. 6 is a graph depicting proliferation of bovine aortic-endothelial cells. In the upper panel, cells were treated with FGF2-K 52-DNA; in the lower panel, cells were treated with a mixture of FGF2, K152, and DNA.

FIG. 7A is a graph displaying P-gal activity after transfection of FGF2/poly-L-lysine/pSVβ-gal into COS cells (lane 2), B16 cells (lane 3), NIH 3T3 cells (lane 4), and DNA alone (lane 1).

FIG. 7B is a graph depicting β-gal expression in COS cells, pSVβ-gal (lanes 1, 3) or pNASSβ-gal (lanes 2, 4) were incubated with (lanes 1, 2) or without (lanes 3, 4) FGF2-K84 and the complexes incubated on COS cells for 48 hrs.

FIG. 7C is a graph showing activity of β-gal activity at various times following transfection with either plasmid alone or with complexes of FGF2/K84/pSV β-gal. -Δ-, DNA alone; -▪-, FGF2-K84-DNA.

FIG. 7D is a graph showing β-gal activity after transfection of various concentrations of FGF2/K84/pSVβ-gal. Lane 1, 0 μg; lane 2, 0.1 μg; lane 3, 1 μg; lane 4, 5 g; lane 5, 10 g.

FIG. 8A is a graph showing β-gal activity in COS cells following transfection of FGF2-K84-pSVβ-gal (lane 1), FGF2+K84+pSVβ-gal (lane 2), FGF2+pSVβ-gal (lane 3), K84+pSVβ-gal (lane 4); pSVβ-gal (lane 5), FGF2-K84 (lane 6), FGF2 (lane 7) and K84 (lane 8).

FIG. 8B is a graph showing competition for cell binding. Lane 1, FGF2-K84-pSVβ-gal complex transfected into COS cells; lane 2, FGF2-K84-pSVβ-gal plus 100 μg FGF2; lane 3, no complex.

FIG. 8C is a graph showing the attenuation of β-gal activity upon the addition of heparin during transfection. Lane 1, FGF2-K84-pSVβ-gal+10 μg heparin; lane 2, FGF2-K84-pSVβ-gal; lane 3, heparin alone; lane 4, pSVβ-gal alone.

FIG. 8D is a graph showing ligand targeting of DNA, pSVβ-gal DNA alone (lane 1), FGF2-K84 (lane 2), histone H1-K84 (lane 3) and cytochrome C-K84 (lane 4) were condensed with pSVβ-gal DNA and added to BHK cells. β-gal activity was measured 48 hr later.

FIG. 9A is a graph showing the effect of chIoroquine on β-gal expression. pSVβ-gal and FGF2-K84 were mixed in the absence (lane 1) or presence (lane 2) of 100 μM chloroquine and incubated for 1 hr at room temperature prior to addition of the complexes to COS cells. Lane 3, chloroquine alone; lane 4, DNA alone.

FIG. 9B is a graph showing the effect of endosome disruptive peptide on β-gal expression. Lane 1, control; lane 2, FGF2-K84-pSVβ-gal; lane 3, FGF2-K84-pSVβ-gal+EDP.

FIG. 9C are photographs of cells stained for β-gal activity following transfection of COS cells with (right panel) or without (left panel) endosome disruptive peptide and FGF2-K84-pSVβ-gal.

FIG. 10 is a photograph of a fluorograph analyzing cell-free translation products. Lane 1, no RNA; lane 2, saporin RNA; lane 3, luciferase RNA; lane 4, saporin RNA and luciferase RNA; lane 5, saporin RNA followed 30 min later with luciferase RNA.

FIG. 11 is a graph depicting direct cytotoxicity of cells transfected by CaPO₄ with an expression vector encoding saporin. Lane 1, mock transfection; lane 2, transfection with pSVβ-gal; lane 3, transfection with saporin-containing vector.

FIG. 12 is a pair of graphs showing cytotoxicity of cells transfected with FGF2-K84-pSVSAP. Left panel, BHK21 cells; right panel, NIH 3T3 cells. Lane 1, FGF2-K84-pSVβ-gal; lane 2, FGF2-K84-pSVSAP.

FIG. 13A is a graph showing β-gal activity with an endosome disruptive peptide in the complex.

FIG. 13B is a graph showing β-gal activity with an endosome disruptive peptide in the complex.

FIG. 13C is a graph showing β-gal activity with an endosome disruptive peptide in the complex.

FIG. 14 is a schematic showing the features of inverse-PCR strategy to create FGF-2 mutants.

FIG. 15 is a graph illustrating the expression levels of β-gal activity in rabbit injured iliac artery.

FIG. 16 is an X-gal stain of arterial segments after delivery of β-gal under control of smooth muscle cell a-actin promoter or no promoter.

FIG. 17 is an X-gal stain of arterial tissue after delivery of P-gal under control of α-actin promoter or no promoter.

FIG. 18 is an antibody stain of arterial tissue after delivery of β-gal under control of a-actin promoter or no promoter.

FIG. 19 is an X-gal stain of arterial segments after delivery of β-gal under control of α-actin promoter or no promoter.

FIG. 20 is a graph illustrating cell death after delivery of DNA by transient transfection encoding either saporin or β-galactosidase under control of SV40 promoter.

FIG. 21 shows tumor weight at 48 hours (upper panel) and 72 hours (lower panel) after delivery of a complex containing DNA encoding β-gal or saporin under control of SV40 promoter or saporin without a promoter or excipient.

FIG. 22 shows tumor weight after two deliveries of a complex containing DNA encoding β-gal or saporin under control of SV40 promoter or saporin without a promoter or excipient.

FIG. 23 shows SDS-PAGE analysis (top) and Western analysis (bottom) of FGF mutants. Upper lanes: lane 1, size markers; lane 2, FGF2; lane 3, lysate of pZ150; lane 4, lysate of R116I; lane 5, lysate of R118K/K119E; lane 6, lysate of Y120A; lane 7, pZ150; lane 8, R1161; lane 9, R118K/K119E; lane 10, Y120A. Lower lanes: lane 1, size markers; lanes 2, 7, 12, R1161; lanes 3, 8, 13, R118K/K119E; lanes 4, 9, 14, Y120A; lanes 5, 10, 15, PZ150.

FIG. 24 is a graph showing the proliferative ability of wild type FGF (FGF150) and FGF mutants on ABAE cells.

### DETAILED DESCRIPTION OF THE INVENTION

Prior to setting forth the invention, it will be helpful to an understanding thereof to define certain terms used herein. The “amino acids,” which occur in the various amino acid sequences appearing herein, are identified according to their well known three letter or one letter abbreviations. The nucleotides, which occur in the various DNA fragments, are designated with the standard single letter designations used routinely in the art.

As used herein, to “bind to a receptor” refers to the ability of a ligand to specifically recognize and detectably bind to a receptor, as assayed by standard in vitro assays. For example, as used herein, binding measures the capacity of an FGF or FGF conjugate to recognize an FGF receptor on a cell, such as a fibroblast, using a procedure substantially as described in Moscatelli, J. Cell Physiol. 131:123-130, 1987.

As used herein, “biological activity” refers to the activity of a compound or a physiological response that results upon in vivo administration of a compound, composition or other mixture. Biological activity thus encompasses therapeutic effects and pharmaceutical activity of such compounds, compositions, complexes, and mixtures. Biological activity may be determined with reference to particular in vitro activities as measured in a defined assay. For example, within the context of this invention, a biological activity of FGF, or fragments of FGF, is the ability of FGF to bind to cells bearing FGF receptors and internalize a linked agent. This activity may be assessed in vitro by conjugating FGF to a cytotoxic agent, such as saporin, contacting cells bearing FGF receptors (e.g., fibroblasts), with the conjugate, and assessing cell proliferation or growth. In vivo activity may be determined using recognized animal models, such as the mouse xenograft model for anti-tumor activity (see, e.g., Beitz et al., Cancer Research 52:227-230, 1992; Houghton et al., Cancer Res. 42:535-539, 1982; Bogden et al., Cancer (Philadelphia) 48:10-20, 1981; Hoogenhout et al., Int. J. Radiat. Oncol, Biol. Phys. 9:871-879, 1983; Stastny et al., Cancer Res. 53:5740-5744, 1993).

As used herein, a “conjugate” refers two or more molecules that are covalently linked together. The molecules may be conjugated directly or through a linker, such as a peptide. A conjugate may be produced by chemical coupling methods or by recombinant expression of chimeric DNA molecules to produce fusion proteins.

A “cytocide-encoding agent” is a nucleic acid molecule that encodes a product that results in cell death and generally acts by inhibiting protein synthesis. Such a product may act by cleaving rRNA or ribonucloprotein, inhibiting an elongation factor, cleaving mRNA, or other mechanism that reduces protein synthesis to a level such that the cell cannot survive. The product may be a protein, ribozyme, antisense, and the like. The nucleic acid molecule may contain additional elements besides the cytocide gene. Such elements include a promoter, enhancer, splice site, transcription terminator, poly(A) signal sequence, bacterial or mammalian origin of replication, selection marker, and the like. The biological activity of a cytocide-encoding agent, may be assayed by any method known to those of skill in the art including, but not limited to, in vitro assays that measure protein synthesis and in vivo assays that assess cytotoxicity by measuring cell proliferation or protein synthesis. Assays that measure cytotoxicity in targeted cells are particularly preferred.

As used herein, the term “cytotoxic agent” refers to a molecule capable of inhibiting cell function. For example, the agent may inhibit proliferation or may be toxic to cells. A variety of cytotoxic agents can be used and include those that inhibit protein synthesis and those that inhibit expression of certain genes essential for cellular growth or survival. Cytotoxic agents include those that result in cell death or that inhibit cell growth, proliferation and/or differentiation. Suitable cytotoxic agents include cytotoxic molecules that inhibit cellular metabolic processes, including transcription, translation, biosynthetic or degradative pathways, DNA synthesis, and other such processes that kill cells, inhibit cell proliferation or make cells recognizable as foreign to the host.

Examples of cytotoxic agents include, but are not limited to, saporin, the ricins, abrin and other ribosome inactivating proteins (RIPs), aquatic-derived cytotoxins, Pseudomonas exotoxin, inhibitors of DNA, RNA or protein synthesis, antisense nucleic acids, other metabolic inhibitors (e.g., DNA cleaving molecules), prodrugs (e.g., thymidine kinase from HSV and bacterial cytosine deaminase), and light-activated porphyrin. While saporin is the preferred RIP, other suitable cytotoxic molecules include ricin, ricin A chain, maize RIP, gelonin, diphtheria toxin, diphtheria toxin A chain, trichosanthin, tritin, pokeweed antiviral protein (PAP), mirabilis antiviral protein (MAP), Dianthins 32 and 30, abrin, monordin, bryodin, shiga, a catalytic inhibitor of protein biosynthesis from cucumber seeds (see, e.g., WO 93/24620), Pseudomonas exotoxin, biologically active fragments of cytotoxins and others known to those of skill in this art.

“Heparin-binding growth factor” refers to any member of a family of heparin-binding growth factor proteins, in which at least one member of the family binds heparin. Preferred growth factors in this regard include FGF, VEGF, and HBEGF. Such growth factors encompass isoforms, peptide fragments derived from a family member, splice variants, and single or multiple exons, some forms of which may not bind heparin.

As used herein, to “hybridize” under conditions of a specified stringency is used to describe the stability of hybrids formed between two single-stranded nucleic acid molecules. Stringency of hybridization is typically expressed in conditions of ionic strength and temperature at which such hybrids are annealed and washed. Typically high, medium and low stringency encompass the following conditions or equivalent conditions thereto: high stringency: 0.1× SSPE or SSC, 0.1% SDS, 65° C.; medium stringency: 0.2× SSPE or SSC, 0.1% SDS, 50° C.; and low stringency: 1.0× SSPE or SSC, 0.1% SDS, 50° C.

“Nucleic acid binding domain” (NABD) refers to a molecule, usually a protein, or peptide (but may also be a polycation) that binds nucleic acids, such as DNA or RNA. An NABD may bind to single or double strands of RNA or DNA or mixed RNA/DNA hybrids. The nucleic acid binding domain may bind to a specific sequence or bind irrespective of the sequence.

As used herein, “nucleic acid” refers to RNA or DNA that is intended for internalization into a cell and includes, but is not limited to, DNA encoding a therapeutic protein, a cytotoxic protein, a prodrug, a ribozyme, or antisense, the complement of these DNAs, an antisense nucleic acid, and other such molecules. Reference to nucleic acids includes duplex DNA, single-stranded DNA, RNA in any form, including triplex, duplex or single-stranded RNA, anti-sense RNA, polynucleotides, oligonucleotides, single nucleotides, chimeras, and derivatives thereof. Nucleic acids may be composed of the well-known deoxyribonucleotides and ribonucleotides (i.e, the bases adenosine, cytosine, guanine, thymidine, and uridine). As well, various other nucleotide derivatives, non-phosphate backbones or phosphate-derived backbones may be used. For example, because normal phosphodiester oligonucleotides (referred to as PO oligonucleotides) are sensitive to DNA- and RNA-specific nucleases, oligonucleotides resistant to cleavage, such as those in which the phosphate group has been altered to a phosphotriester, methylphosphonate, or phosphorothioate may be used (see U.S. Pat. No. 5,218,088).

As used herein, “operative linkage” or operative association of two nucleotide sequences refers to the functional relationship between such sequences. Nucleotide sequences include, but are not limited to, DNA encoding a product, DNA encoding a signal sequence, promoters, enhancers, transcriptional and translational stop sites, and polyadenylation signals. For example, operative linkage of DNA encoding a cytocide to a promoter refers to the physical and functional relationship between the DNA and the promoter such that transcription of the DNA is initiated from the promoter by an RNA polymerase that specifically recognizes, binds to, and transcribes the DNA.

As used herein, the phrase “polypeptide reactive with an FGF receptor” refers to any polypeptide that specifically interacts with an FGF receptor, preferably the high affiity FGF receptor, and is transported into the cell by virtue of its interaction with the FF receptor.

As used herein, a “prodrug” is a compound that metabolizes or otherwise converts an inactive, nontoxic compound to a biologically, pharmaceutically, therapeutically, or toxic active form of the compound. A prodrug may also be a pharmaceutically inactive compound that is modified upon administration to yield an active compound through metabolic or other processes. By virtue of knowledge of pharmacodynamic processes and drug metabolism in vivo, once a pharmaceutically active compound is known inactive forms of the compound may be synthesized or isolated (see, e.g., Nogrady, Medicinal Chemistry A Biochemical Approach, Oxford University Press, New York, pages 388-392, 1985).

As used herein, “receptor-binding internalized ligand” or “ligand” refers to any peptide, polypeptide, protein or non-protein, such as a peptidomimetic, that is capable of binding to a cell-surface molecule and internalization. Within the context of this invention, the receptor-binding internalized ligand is conjugated to a nucleic acid binding domain, either as a fusion protein or through chemical conjugation, and is used to deliver a cytocide-encoding or prodrug encoding agent to a cell. In one aspect, the ligand is directly conjugated to a nucleic acid molecule, which may be further complexed with a nucleic acid binding domain. Such ligands include growth factors, cytokines, antibodies, hormones, and the like.

As used herein, “saporin” (abbreviated herein as SAP) refers to a polypeptide isolated from the leaves or seeds of Saponaria officinalis, as well as modified forms that have amino acid substitutions, deletions, insertions or additions, which express substantial ribosome inactivating activity. Purified preparations of saporin are frequently observed to include several molecular isoforms of the protein. It is understood that differences in amino acid sequences can occur in saporin from different species as well as between saporin molecules from individual organisms of the same species. Saporin for use herein may be purified from leaves, chemically synthesized, or synthesized by expression of DNA encoding a saporin polypeptide.

As used herein, a “targeted agent” is a nucleic acid molecule that is internally delivered to a cell by a receptor-binding internalized ligand, and upon internalization alters or affects cellular metabolism, growth, activity, viability or other property or characteristic of the cell.

As used herein, a “therapeutic nucleic acid” describes any nucleic acid molecule used in the context of the invention that effects a treatment, generally by modifying gene transcription or translation. It includes, but is not limited to, the following types of nucleic acids: nucleic acids encoding a protein, antisense RNA, DNA intended to form triplex molecules, protein binding nucleic acids, and small nucleotide molecules. A therapeutic nucleic acid may be used to effect genetic therapy by serving as a replacement for a defective gene, by encoding a therapeutic product or by encoding a cytotoxic molecule, especially an enzyme, such as saporin. The therapeutic nucleic acid may encode all or a portion of a gene, and may function by recombining with DNA already present in a cell, thereby replacing a defective portion of a gene. It may also encode a portion of a protein and exert its effect by virtue of co-suppression of a gene product.

Preparation of Ligand-directed Nucleic Acid Delivery Vehicles

As noted above, the present invention provides nucleic acids, such as cytocide-encoding or prodrug-encoding agents, (a) complexed with a conjugate of a receptor-binding internalized ligand and a nucleic acid binding domain, or (b) conjugated to a ligand. Upon binding to an appropriate receptor, the nucleic acid-ligand complex or conjugate is internalized and trafficked through the cell via the endosomal compartment, where at least a portion of the complex may be cleaved.

I. Ligands

A. Ligands that Bind Tumor Cells

As noted above, receptor-binding internalized ligands are used to deliver nucleic acids, including a cytocide-encoding agent, to a cell expressing an appropriate receptor on its cell surface. Numerous molecules that bind specific receptors on tumor cells have been identified and are suitable for use in the present invention. For example, the following table sets forth some of the better known ligands and cell surface molecules on various tumors.

| Tumor | Ligand | Receptor |
|---|---|---|
| T cell lymphomas | IL-2 | IL-2 receptor |
| B cell lymphomas | Antibody | Immunoglobulin idiotypes |
| Melanomas | FGF | MAGE; FGF receptor |
| Prostate tumors | | Prostate specific antigen-1; |
| | | probasin |
| Angiogenic tumors | FGF; VEGF; PDGF | FGF receptor; VEGF |
| | | receptor; PDGF receptor |
| Breast tumors | heregulin; FGF | erb B2; erb B3; erb B4; |
| | | MUC-1; HSP-27; int-1; int-2 |
| Colon, lung tumors | Antibody; FGF; | CEA; FGF receptor; VEGF |
| | VEGF | receptor |
| Bladder tumors | HBEGF; EGF/TGF; | EGF receptor, FGF receptor |
| | FGF | |
| Pancreatic tumors | FGF | FGF receptor |
| Myeloid luekemias | FGF; CD antibodies | FGF receptor; CD molecules |
| Endometrial | VEGF | VEGF receptor |
| carcinoma; cervical | | |
| carcinoma | | |

In addition, other receptors, such as transferrin receptor, are preferentially expressed on most all tumor cells. Antibodies that are specific to cell surface molecules on tumors are readily generated as monoclonals or polyclonal antisera. Many such antibodies are available (e.g., from American Type Culture Collection, Rockville, Md.).

Fragments of these ligands may be used within the present invention, so long as the fragment retains the ability to bind to the appropriate cell surface molecule. Likewise, ligands with substitutions or other alterations, but which retain binding ability, may also be used. As well, a particular ligand refers to a polypeptide(s) having an amino acid sequence of the native ligand, as well as modified sequences, (eg., having amino acid substitutions, deletions, insertions or additions compared to the native protein) as long as the ligand retains the ability to bind to its receptor on a tumor cell and be internalized.

Ligands also encompass muteins that possess the ability to bind to its receptor expressing cells and be internalized. Such muteins include, but are not limited to, those produced by replacing one or more of the cysteines with serine as described herein. Typically, such muteins will have conservative amino acid changes. DNA encoding such muteins will, unless modified by replacement of degenerate codons, hybridize under conditions of at least low stringency to native DNA sequence encoding the wild-type ligand.

DNA encoding a ligand may be prepared synthetically based on known amino acid or DNA sequence, isolated using methods known to those of skill in the art (e.g., PCR amplification), or obtained from commercial or other sources. DNA encoding a ligand may differ from the above sequences by substitution of degenerate codons or by encoding different amino acids. Differences in amino acid sequences, such as those occurring among the homologous ligand of different species as well as among individual organisms or species, are tolerated as long as the ligand binds to its receptor. Ligands may be isolated from natural sources or made synthetically, such as by recombinant means or chemical synthesis.

1. Fibroblast Growth Factors

One family of factors that may be used within the context of the present invention is the fibroblast growth factor (FGF) family. The members of the FGF family have a high degree of amino acid sequence similarities and common physical and biological properties, including the ability to bind to one or more FGF receptors.

This family of proteins includes FGFs designated FGF-1 (acidic FGF (aFGF)), FGF-2 (basic FGF (bFGF)), FGF-3 (int-2) (see, e.g., Moore et al., EMBO J. 5:919-924, 1986), FGF-4 (hst-1/K-FGF) (see, e.g., Sakamoto et al., Proc. Natl. Acad Sci. USA 86:1836-1840, 1986; U.S. Pat. No. 5,126,323), FGF-5 (see, e.g., U.S. Pat. No. 5,155,217), FGF-6 (hst-2) (see, e.g., published European Application EP 0 488 196 A2; Uda et al., Oncogene 7:303-309, 1992), FGF-7 (keratinocyte growth factor) (KGF) (see, e.g., Finch et al., Science 245:752-755, 1985; Rubin et al., Proc. Natl. Acad Sci USA 86:802-806, 1989; and International Application WO 90/08771), FGF-8 (see, e.g., Tanaka et al., Proc Natl. Acad. Sci. USA 89:8528-8532, 1992); FGF-9 (see, Miyamoto et al., Mol. Cell. Biol. 13:4251-4259, 1993); FGF-11, FGF-13; FGF14; and FGF15. The members of the FGF family have a high degree of amino acid sequence similarities and common physical and biological properties, including the ability to bind to one or more FGF receptors.

DNA encoding FGF peptides and/or the amino acid sequences of FGFs are well known. For example, DNA encoding human FGF-1 (Jaye et al., Science 233:541-545, 1986; U.S. Pat. No. 5,223,483), bovine FGF-2 (Abraham et al., Science 233:545-548, 1986; Esch et al., Proc. Natl. Acad. Sci. USA 82:6507-6511, 1985; and U.S. Pat. No. 4,956,455), human FGF-2 (Abraham et al., EMBO J. 5:2523-2528, 1986; U.S. Pat. No. 4,994,559; U.S. Pat. No. 5,155,214; EP 470,183B; and Abraham et al., Quant. Biol. 51:657-668, 1986) rat FGF-2 (see Shimasaki et al., Biochem. Biophys. Res. Comm., 1988, and Kurokawa et al., Nucleic Acids Res. 16:520 1, 1988), FGF-3, FGF-6, FGF-7 and FGF-9 are known (see also U.S. Pat. No. 5,155,214; U.S. Pat. No.4,956,455; U.S. Pat. No. 5,026,839; U.S. Pat. No. 4,994,559, EP 0,488,196 A2, EMBL or GenBank databases, and references discussed herein) as well as FGF-11 (WO 96/39507); FGF-13 (WO 96/39508); FGF-14 (WO 96/39506); and FGF-15 (WO 96/39509).

FGFs exhibit a mitogenic effect on a wide variety of mesenchymal, endocrine and neural cells and stimulate collateral vascularization and angiogenesis. In some instances, FGF-induced mitogenic stimulation may be detrimental. For example, cell proliferation and angiogenesis are an integral aspect of tumor growth. Members of the FGF family, including FGF-2, are thought to play a pathophysiological role in many diseases. To reduce or eliminate mitogenesis, muteins of FGF are constructed as described below. Such muteins retain the ability to bind to high and low affinty receptors.

The effects of FGFs are mediated by high affinity receptor tyrosine kinases present on the cell surface of FGF-responsive cells (see, e.g., PCT WO 91/00916, WO 90/05522, PCT WO 92/12948; Imamura et al., Biochem. Biophys. Res. Comm. 155:583-590, 1988; Huang et al., J. Biol. Chem. 261:9568-9571, 1986; Partanen et al., EMBO J. 10:1347, 1991; and Moscatelli, J. Cell. Physiol. 131:123, 1987). Low affinity receptors also appear to play a role in mediating FGF activities. The high affinity receptor proteins are single chain polypeptides with molecular weights ranging from 110 to 150 kD, depending on cell type that constitute a family of structurally related FGF receptors. Four FGF receptor genes have been identified, three of which generate multiple mRNA transcripts via alternative splicing of the primary transcript. Some receptor specificity has been uncovered. For example, FGF-9 binds specifically to FGFR3, which is expressed in epithelial cells and cartilage rib bone, epithelial cells exclusively express FGFR3IIIb, while mesenchymal cells express FGFR3IIIb and FGFR3IIIc.

2. Vascular Endothelial Growth Factors

Vascular endothelial growth factors (VEGF) can directly stimulate endothelial cell growth, enhance angiogenesis, enhance glucose transport, as well as cause a rapid and reversible increase in blood vessel permeability. VEGF is expressed during normal development and in certain normal adult organs. Purified VEGF is a basic, heparin-binding, homodimeric glycoprotein that is heat-stable, acid-stable and may be inactivated by reducing agents.

The members of this family have been referred to variously as vascular endothelial growth factor (VEGF), vascular permeability factor (VPF) and vasculotropin (see, e.g., Plouet et al., EMBO J. 8:3801-3806, 1989). Herein, they are collectively referred to as VEGF.

DNA sequences encoding VEGF may be found in U.S. Pat. No. 5,240,848, U.S. Pat. No. 5,332,671, U.S. Pat. No. 5,219,739, U.S. Pat. No. 5,194,596, Houch et al., Mol. Endocrin. 5:180, 1991; and GenBank nucleotide database.

Four molecular species of VEGF result from alternative splicing of mRNA and contain 121, 165, 189 and 206 amino acids. The predominant isoform secreted by a variety of normal and transformed cells is VEGF₁₆₅. The secreted isoforms, VEGF₁₂₁ and VEGF₁₆₅ are preferred VEGF proteins. The longer isoforms, VEGF₁₈₉ and VEGF₂₀₆, bind to the extracellular matrix and need to be released by an agent, such as suramin, heparin or heparinase, or plasmin. Other preferred VEGF proteins contain various combinations of VEGF exons, such that the protein still binds VEGF receptor and is internalized.

It is not necessary that a VEGF protein used in the context of this invention either retain any of its in vivo biological activities, such as stimulating endothelial cell growth, or bind heparin other than bind a VEGF receptor on a cell and be internalized. However, it may be desirable in certain contexts for VEGF to manifest certain of its biological activities. For example, if VEGF is used as a carrier for DNA encoding a molecule useful in wound healing, it would be desirable that VEGF exhibit vessel permeability activity and promotion of fibroblast migration and angiogenesis. It will be apparent from the teachings provided within the subject application which of the activities of VEGF are desirable to maintain.

Quiescent and proliferating endothelial cells bind VEGF with high-affinity, and endothelial cell responses to VEGF appear to be mediated by high affinity cell surface receptors (see, e.g., PCT Application WO 92/14748, U.S. application Ser. No. 08/657,236, de Vries et al., Science 255:989-91, 1992; Terman et al., Biochem. Biophys. Res. Commun. 187:1579-1586, 1992; Kendall et al., Proc. Natl. Acad. Sci. USA90:10705-10709, 1993; and Peters et al., Proc. Natl. Acad. Sci USA 90:8915-8919, 1993). Two tyrosine kinases have been identified as VEGF receptors. The first, known as fms-like tyrosine kinase or FLT, is a receptor tyrosine kinase that is specific for VEGF. In adult and embryonic tissues, expression of FLT mRNA is localized to the endothelium and to populations of cells that give rise to endothelium. The second receptor, KDR (human kinase insert domain-containing receptor), and its mouse homologue FLK-1, are closely related to FLT. The KDR/FLK-1 receptor is expressed in endothelium during the fetal growth stage, during earlier stages of embryonic development, and in adult tissues. In addition, messenger RNA encoding FLT and KDR have been identified in tumor blood vessels and specifically by endothelial cells of blood vessels supplying glioblastomas. Similarly, FLT and KDR mRNAs are upregulated in tumor blood vessels in invasive human colon adenocarcinoma, but not in the blood vessels of adjacent normal tissues.

3. Heparin-binding Epidermal Growth Factors

HBEGF interacts with the same high affinity receptors as EGF on bovine aortic smooth muscle cells and human A43 1 epidermoid carcinoma cells (Higashiyama, Science 251:936-939, 1991). HBEGFs exhibit a mitogenic effect on a wide variety of cells including BALB/c 3T3 fibroblast cells and smooth muscle cells, but are not mitogenic for endothelial cells (Higashiyama et al., Science 251:936-939, 1991). However, HBEGF has a stimulatory effect on collateral vascularization and angiogenesis. Members of the HBEGF family are thought to play a pathophysiological role, for example, in a variety of tumors, such as bladder carcinomas, breast tumors and non-small cell lung tumors. Thus, these cell types are likely candidates for delivery of cytocide-encoded agents.

HBEGF isolated from U-937 cells is heterogeneous in structure and contains at least 86 amino acids and two sites of O-linked glycosyl groups (Higashiyama et al., J. Biol. Chem. 267:6205-6212, 1992). The carboxyl-terminal half of the secreted HBEGF shares approximately 35% sequence identity with human EGF, including six cysteines spaced in the pattern characteristic of members of the EGF protein family. In contrast, the amino-terminal portion of the mature factor is characterized by stretches of hydrophilic residues and has no structural equivalent in EGF. Site-directed mutagenesis of HBEGF and studies with peptide fragments have indicated that the heparin-binding sequences of HBEGF reside primarily in a 21 amino acid stretch upstream of and slightly overlapping the EGF-like domain.

For the purposes of this invention, HBEGF need only bind a specific HBEGF receptor and be internalized. Members of the HBEGF family are those that have sufficient nucleotide identity to hybridize under normal stringency conditions (typically greater than 75% nucleotide identity). Subfragments or subportions of a full-length HBEGF may also be desirable. One skilled in the art may find from the teachings provided within that certain biological activities are more or less desirable, depending upon the application.

DNA encoding an HBEGF peptide or polypeptide refers to any DNA fragment encoding an HBEGF, HBEGF fragment or HBEGF mutein that binds an EGF receptor and internalizes. Such DNA sequences encoding HBEGF fragments are available from publicly accessible databases, such as: EMBL, GenBank (Accession Nos. M93012 (monkey) and M60278 (human)); the plasmid pMTN-HBEGF (ATCC #40900) and pAX-HBEGF (ATCC #40899) (described in PCT Application WO/92106705); and Abraham et al., Biochem. Biophys. Res. Comm. 190:125-133, 1993).

The effects of HBEGFs are mediated by EGF receptor tyrosine kinases expressed on cell surfaces of HBEGF-responsive cells (see, e.g., U.S. Pat. Nos. 5,183,884 and 5,218,090; and Ullrich et al., Nature 309:4113-425, 1984). The EGF receptor proteins, which are single chain polypeptides with molecular weights 170 kD, constitute a family of structurally related EGF receptors. Cells known to express the EGF receptors include smooth muscle cells, fibroblasts, keratinocytes, and numerous human cancer cell lines, such as the: A431 (epidermoid); KB3-1 (epidermoid); COLO 205 (colon); CRL 1739 (gastric); HEP G2 (hepatoma); LNCAP (prostate); MCF-7 (breast); MDA-MB-468 (breast); NCI 417D (lung); MG63 (osteosarcoma); U-251 (glioblastoma); D-54MB (glioma); and SW-13 (adrenal).

4. Antibodies to Molecules on Tumor Cells

Antibodies to molecules expressed on the surface of tumor cells are useful within the context of the present invention as long as the antibody is internalized following binding. Such antibodies include but are not limited to antibodies to FGF receptors, VEGF receptors, and the receptors set forth above.

Antibodies may be polyclonal or monoclonal. Commercially available antibodies to some tumor cell surface molecules may be used if they internalize. Briefly, antibodies are raised by immunization of mice, rats, rabbits or other animals with tumor cells. Various immunization protocols may be found in for example, Harlow and Lane (Antibodies: A Laboratory Manual, Cold Spring Harbor Laboratory, 1988) and Coligan et al. (Current Protocols in Immunology, Greene Publishing, 1995). Following immunization, spleen or lymph nodes are collected for generating hybridomas or serum is collected for polyclonal antibodies. Hybridomas are preferred. Cells from spleen or lymph node are fused to a myeloma cell line (see, Harlow and Lane, supra; and Coligan et al., supra; for protocols). Antibody-secreting hybridomas are grown, and the antibodies are tested for binding to tumor cells by ELISA, section staining, flow cytometry, confocal microscopy and the like. Preferably the antibody does not bind or binds much less to counterpart normal cells. Positive antibodies are further tested for internalization. One assay that is used is a test for an antibody to kill tumor cells. Briefly, the test hybridoma antibody and tumor cells are incubated. Unbound antibody is washed away. A second stage antibody, such as an anti-IgG antibody, conjugated to saporin is incubated with the tumor cells. Cell killing is assessed by any known assay, including trypan blue exclusion, MTT uptake, fluorescein diacetate staining, and the like.

5. Other Ligands that Bind to Tumor Cells

Other receptor-binding ligands may be used in the present invention. Any protein, polypeptide, analogue, or fragment that binds to a cell-surface receptor and is internalized may be used. These ligands may be produced by recombinant or other means in preparation for conjugation to the nucleic acid binding domain. The DNA sequences and methods to obtain the sequences of these receptor-binding internalized ligands are well known. For example, these ligands include CSF-1 (GenBank Accession Nos. M11038, M37435; Kawasaki et al., Science 230:291-296, 1985; Wong et al., Science 235:1504-1508, 1987); GM-CSF (GenBank Accession No. X03021; Miyatake et al., EMBO J. 4:2561-2568, 1985); IFN-α (interferon) (GenBank Accession No. A02076; Patent No. WO 8502862-A, Jul. 4, 1985); IFN-γ (GenBank Accession No. A02137; Patent No. WO 8502624-A, Jun. 20, 1985); IL-1-α (interleukin 1 alpha) (GenBank Accession No. X02531, M15329; March et al., Nature 315:641-647, 1985; Nishida et al., Biochem. Biophys. Res. Commun. 143:345-352, 1987); IL-1-β (interleukin 1 beta) (GenBank Accession No. X02532, M15330, M15840; March et al., Nature 315:641-647, 1985; Nishida et al., Biochem. Biophys. Res. Commun. 143:345-352, 1987; Bensi et al., Gene 52:95-101, 1987); IL-I (GenBank Accession No. K02770, M54933, M38756; Auron et al., Proc. Natl. Acad. Sci. USA 81:7907-7911, 1984; Webb et al., Adv. Gene Technol. 22:339-340, 1985); IL-2 (GenBank Accession No. A14844, A21785, X00695, X00200, X00201, X00202; Lupker et al., Patent No. EP 0307285-A, March 15, 1989; Perez et al., Patent No. EP 0416673-A, March 13, 1991; Holbrook et al., Nucleic Acids Res. 12:5005-5013, 1984; Degrave et al., EMBO J. 2:2349-2353, 1983; Taniguchi et al., Nature 302:305-310, 1983); IL-3 (GenBank Accession No. M14743, M20137; Yang et al., Cell 47:3-10, 1986; Otsuka et al., J. Immunol. 140:2288-2295, 1988); IL-4 (GenBank Accession No. M13982; Yokota et al., Proc. Natl. Acad Sci. USA 83:5894-5898, 1986); IL-5 (GenBank Accession No. X04688, J03478; Azuma et al., Nucleic Acids Res. 14:9149-9158, 1986; Tanabe et al., J. Biol. Chem. 262:16580-16584, 1987); IL-6 (GenBank Accession No. Y00081, X04602, M54894, M38669, M14584; Yasukawa et al., EMBO J. 6:2939-2945, 1987; Hirano et al., Nature 324:73-76, 1986; Wong et al., Behring Inst. Mitt. 83:4047, 1988; May et al., Proc. Natl. Acad. Sci. USA 83:8957-8961, 1986); IL-7 (GenBank Accession No. J04156; Goodwin et al., Proc. Natl. Acad. Sci. USA 86:302-306, 1989); IL-8 (GenBank Accession No. Z11686; Kusner et al., Kidney Int. 39:1240-1248, 1991); IL-10 (GenBank Accession No. X78437, M57627; Vieira et al., Proc. Natl. Acad Sci. USA 88:1172-1176, 1991); IL-11 (GenBank Accession No. M57765 M37006; Paul et al., Proc. Natl. Acad. Sci. USA 87:7512-7516, 1990); IL-13 (GenBank Accession No. X69079, U10307; Minty et al., Nature 362:248-250, 1993; Smimov, Shemyakin and Ovchinnikov Institute of Bioorganic Chemistry, Jun. 2, 1994); TNF-α (Tumor necrosis factor) (GenBank Accession No. A21522; Patent No. GB 2246569-A1, Feb. 5, 1992); TNF-β (GenBank Accession No. D12614; Matsuyama et al., FEBS LETTERS 302:141-144, 1992); urokinaselurokinase receptor (GenBank Accession Nos. X02760/X74309); α-1,3 fucosyl transferase, α1-antitrypsin/E-selectin (GenBank Accession Nos. M98825, D382571M87862); P-selectin glycoprotein ligand, P-selectin ligand/P-selectin (GenBank Accession Nos. U25955, U02297/ L01574); VCAM1/VLA-4 integrin receptor (GenBank Accession Nos. X53051/X16983 and L12002); E9 (Blann et al., Atherosclerosis 120:221, 1996)/TGFβ receptor; Fibronectin (GenBank Accession No. X02761);type I^{α1} collagen (GenBank Accession No. Z74615), type I β2-collagen (GenBank Accession No. Z74616), hyaluronic acid/CD44 (GenBank Accession No. M59040); CD40 ligand (GenBank Accession No. L07414)/CD40 (GenBank Accession No. M83312); EFL-3, LERTK-2 ligands (GenBank Accession Nos. L37361, U09304) for elk-i (GenBank Accession No. M25269); VE-cadherin (GenBank Accession No. X7998 1) ligand for catenins; ICAM-3 (GenBank Accession No. X69819) ligand for LFA-1, and von Willebrand Factor (GenBank Accession No. X04385), fibrinogen and fibronectin (GenBank Accession No. X92461 ligands for αᵥβ₃ integrin (GenBank Accession Nos. U07375, L28832) and GP30 ligand (S68256) for erbB2. DNA sequences of other suitable receptor-binding internalized ligands may be obtained from GenBank or EMBL DNA databases, reverse-synthesized from protein sequence obtained from PIR database or isolated by standard methods (Sambrook et al., supra) from cDNA or genomic libraries.

6. Selection of Other Ligands that Bind Tumor Cell Surface Molecules

Ligands for use in the present invention may also be selected by a method such as phage display (see, for example, U.S. Pat. No. 5,223,409.) Briefly, in this method, DNA sequences are inserted into the gene III or gene VIII gene of a filamentous phage, such as Ml 3. Several vectors with multicloning sites have been developed for insertion (McLafferty et al., Gene 128:29-36, 1993; Scott and Smith, Science 249:386-390, 1990; Smith and Scott, Methods Enzymol. 217:228-257, 1993). The inserted DNA sequences may be randomly generated or be variants of a known binding domain for binding tumor cells. Single chain antibodies may readily be generated using this method. Generally, the inserts encode from 6 to 20 amino acids. The peptide encoded by the inserted sequence is displayed on the surface of the bacteriophage. Bacteriophage expressing a binding domain for tumor cells are selected for by binding to tumor cells. Unbound phage are removed by a wash, typically containing 10 mM Tris, 1 mM EDTA, and without salt or with a low salt concentration. Bound phage are eluted with a salt containing buffer, for example. The NaCl concentration is increased in a step-wise fashion until all the phage are eluted. Typically, phage binding with higher affinity will be released by higher salt concentrations. Eluted phage are propagated in the bacteria host. Further rounds of selection may be performed to select for a few phage binding with high affinity. The DNA sequence of the insert in the binding phage is then determined. Once the predicted amino acid sequence of the binding peptide is known, sufficient peptide for use herein as an nucleic acid binding domain may be made either by recombinant means or synthetically. Recombinant means is used when the receptor-binding internalized ligand/nucleic acid binding domain is produced as a fusion protein. The peptide may be generated as a tandem array of two or more peptides, in order to maximize affity or binding.

B. Modifications of Receptor-binding Internalized Ligands

The ligands for use herein may be customized for a particular application. Means for modifying proteins is provided below. Briefly, additions, substitutions and deletions of amino acids may be produced by any commonly employed recombinant DNA method. Modified peptides, especially those lacking proliferative function, and chimeric peptides, which retain the specific binding and internalizing activities are also contemplated for use herein.

As noted above, any ligand that binds to a tumor cell surface receptor and is internalized may be used within the context of this invention. Such ligands may be polypeptides or peptide analogues, including peptidomimetics. Ligands also include fragments thereof, or constrained analogues of such peptides that bind to the receptor and internalize a linked targeted agent. Members of the FGF family, including FGF-1 to FGF-9, are preferred.

A modification that is effected substantially near the N-terminus of a polypeptide is generally effected within the first about ten to twenty residues of the protein. Such modifications include the addition or deletion of residues, such as the addition of a cysteine to facilitate conjugation and to form conjugates that contain a defined molar ratio of 1:1 of the polypeptides. An amino acid residue of a receptor-binding internalized ligand is non-essential if the polypeptide that has been modified by deletion or alteration of the residue possesses substantially the same ability to bind to its receptor and internalize a linked agent as the unmodified polypeptide.

Modification of the polypeptide may be effected by any means known to those of skill in this art. The preferred methods herein rely on modification of DNA encoding the polypeptide and expression of the modified DNA. DNA encoding one of the receptor-binding internalized ligands discussed above may be mutagenized using standard methodologies. For example, cysteine residues that are responsible for aggregate formation may be deleted or replaced. If necessary, the identity of cysteine residues that contribute to aggregate formation may be determined empirically, by deleting and/or replacing a cysteine residue and ascertaining whether the resulting protein aggregates in solutions containing physiologically acceptable buffers and salts. In addition, fragments of these receptor-binding internalized ligands may be constructed and used. The binding region of many of these ligands have been delineated. The receptor binding region of FGF2 has been identified by mutation analysis and FGF peptide agonists/antagonists to reside between residues 33-77 and between 102-129 of the 155 amino acid form (Baird et al., PNAS 85:2324; Erickson et al., Biochem. 88:3441). Exons 1-4 of VEGF are required for receptor binding. The C-terminal portion of HBEGF has been predicted to be involved in receptor binding. However, in addition to the C-terminal portion, loop A and loop C are required for binding. Mutation of either residue 42 (Arg) or residue 48 (Leu) of HBEGF abolish receptor binding. These two residues and the loop structure are conserved between EGF family members.

Mutations may be made by any method known to those of skill in the art, including site-specific or site-directed mutagenesis of DNA encoding the protein and the use of DNA amplification methods using primers to introduce and amplify alterations in the DNA template, such as PCR splicing by overlap extension (SOE). Site-directed mutagenesis is typically effected using a phage vector that has single- and double-stranded forms, such as M13 phage vectors, which are well-known and commercially available. Other suitable vectors that contain a single-stranded phage origin of replication may be used (see, e.g., Veira et al., Meth. Enzymol. 15:3, 1987). In general, site-directed mutagenesis is performed by preparing a single-stranded vector that encodes the protein of interest (i.e., a member of the FGF family or a cytotoxic molecule, such as a saporin). An oligonucleotide primer that contains the desired mutation within a region of homology to the DNA in the single-stranded vector is annealed to the vector followed by addition of a DNA polymerase, such as E. coli DNA polymerase I (Klenow fragment), which uses the double stranded region as a primer to produce a heteroduplex in which one strand encodes the altered sequence and the other the original sequence. The heteroduplex is introduced into appropriate bacterial cells and clones that include the desired mutation are selected. The resulting altered DNA molecules may be expressed recombinantly in appropriate host cells to produce the modified protein.

Suitable conservative substitutions of amino acids are well-known and may be made generally without altering the biological activity of the resulting molecule. For example, such substitutions are generally made by interchanging within the groups of polar residues, charged residues, hydrophobic residues, small residues, and the like. If necessary, such substitutions may be determined empirically merely by testing the resulting modified protein for the ability to bind to and internalize upon binding to the appropriate receptors. Those that retain this ability are suitable for use in the conjugates and methods herein. In addition, muteins of the FGFs are known to those of skill in the art (see, e.g., U.S. Pat. No. 5,175,147; PCT Application No. WO 89/00198, U.S. Ser. No. 07/070,797; PCT Application No. WO 91/15229; and U.S. Ser. No. 07/505,124).

Binding to a receptor followed by internalization are the only activities required for a ligand to be suitable for use herein. However, some of the ligands are growth factors and cause mitogenesis. For example, all of the FGF proteins induce mitogenic activity in a wide variety of normal diploid mesoderm-derived and neural crest-derived cells. A test of such “FGF mitogenic activity”, which reflects the ability to bind to FGF receptors and to be internalized, is the ability to stimulate proliferation of cultured bovine aortic endothelial cells (see, e.g., Gospodarowicz et al., J. Biol. Chem. 257:12266-12278, 1982; Gospodarowicz et al., Proc. Natl. Acad. Sci. USA 73:4120-4124, 1976). Muteins with reduced mitogenic activity are made by the methods described herein. In the Examples, FGF muteins with reduced mitogenic activity have been constructed by site-directed mutagenesis. Non- or reduced-mitogenic proteins can also be constructed by swapping the receptor-binding domain with the receptor-binding domain of a related protein. By way of example, the domain of FGF2 may be swapped with the receptor-binding domain of FGF7 to create an FGF that does not cause proliferation and may alter the binding profile.

If the FGF or other ligand has been modified so as to lack mitogenic activity or other biological activities, binding and internalization may still be readily assayed by any one of the following tests or other equivalent tests. Generally, these tests involve labeling the ligand, incubating it with target cells, and visualizing or measuring intracellular label. For example, briefly, FGF may be fluorescently labeled with FITC or radiolabeled with 1251. Fluorescein-conjugated FGF is incubated with cells and examined microscopically by fluorescence microscopy or confocal microscopy for internalization. When FGF is labeled with ¹²⁵I, the labeled FGF is incubated with cells at 4° C. Cells are temperature shifted to 37° C. and washed with 2 M NaCl at low pH to remove any cell-bound FGF. Label is then counted and thereby measuring internalization of FGF. Alternatively, the ligand can be conjugated with an nucleic acid binding domain by any of the methods described herein and complexed with a plasmid encoding saporin or conjugated with saporin or other cytotoxic molecule and assessed for cytotoxicity. As discussed below, the complex may be used to transfect cells and cytotoxicity measured.

II. Nucleic Acid Binding Domains

As previously noted, nucleic acid binding domains (NABD) interact with the target nucleic acid either in either a sequence-specific manner or a sequence-nonspecific manner. When the interaction is non-specific, the nucleic acid binding domain binds nucleic acid regardless of its sequence. For example, poly-L-lysine or poly-D lysine is a basic polypeptide that binds to oppositely charged DNA. Other highly basic proteins or polycationic compounds, such as histones, protamines, and spermine and spermidine, also bind to nucleic acids in a nonspecific manner. In addition, MnCl₂ and cobalt hexamine also bind DNA and may serve to condense nucleic acid.

Many proteins have been identified that bind specific sequences of DNA. These proteins are responsible for genome replication, transcription and repair of damaged DNA. The transcription factors regulate gene expression and are a diverse group of proteins. These factors are especially well suited for purposes of the subject invention because of their sequence-specific recognition. Host transcription factors have been grouped into seven well-established classes based upon the structural motif used for recognition. The major families include helix-turn-helix (HTH) proteins, homeodomains, zinc finger proteins, steroid receptors, leucine zipper proteins, the helix-loop-helix (HLH) proteins, and β-sheets. Other classes or subclasses may eventually be delineated as more factors are discovered and defined. Proteins from those classes or proteins that do not fit within one of these classes but bind nucleic acid in a sequence-specific manner, such as SV40 T antigen and p53 may also be used.

These families of transcription factors are generally well-known (see GenBank; Pabo and Sauer, Ann. Rev. Biochem. 61:1053-1095, 1992; and references below). Many of these factors are cloned and the precise DNA-binding region delineated in certain instances. When the sequence of the DNA-binding domain is known, a gene encoding it may be synthesized if the region is short Alternatively, the genes may be cloned from the host genomic libraries or from CDNA libraries using oligonucleotides as probes or from genomic DNA or cDNA by polymerase chain reaction methods. Such methods may be found in Sambrook et al., supra.

Helix-turn-helix proteins include the well studied λ Cro protein, λcI, and E. coli CAP proteins (see Steitz et al., Proc. Natl. Acad. Sci. USA 79:3097-3100, 1982; Ohlendorf et al., J. Mol. Biol. 169:757-769, 1983). In addition, the lac repressor (Kaptein et al., J. Mol. Biol. 182:179-182, 1985) and Trp repressor (Scheritz et al., Nature 317:782-786, 1985) belong to this family. Members of the homeodomain family include the Drosophila protein Antennapaedia (Qian et al., Cell. 59:573-580, 1989) and yeast MATα2 (Wolberger et al., Cell. 67:517-528, 1991). Zinc finger proteins include TFIIIA (Miller et al., EMBO J. 4:1609-1614, 1985), Sp-1, zif 268, and many others (see generally Krizek et al., J. Am. Chem. Soc. 113:4518-4523, 1991). Steroid receptor proteins include receptors for steroid hormones, retinoids, vitamin D, thyroid hormones, as well as other compounds. Specific examples include retinoic acid, knirps, progesterone, androgen, glucocosteroid and estrogen receptor proteins. The leucine zipper family was defined by a heptad repeat of leucines over a region of 30 to 40 residues. Specific members of this family include C/EBP, c-fos, c-jun, GCN4, sis-A, and CREB (see generally O'Shea et al., Science 254:539-544, 1991). The helix-loop-helix (HLH) family of proteins appears to have some similarities to the leucine zipper family. Well-known members of this family include myoD (Weintraub et al., Science 251:761-766, 1991); c-myc; and AP-2 (Williams and Tijan, Science 251:1067-1071, 1991). The β-sheet family uses an antiparallel β-sheet for DNA binding, rather than the more common α-helix. The family contains the MetJ (Phillips, Curr. Opin. Struc. Biol. 1:89-98, 1991), Arc (Breg et al., Nature 346:586-589, 1990) and Mnt repressors. In addition, other motifs are used for DNA binding, such as the cysteine-rich motif in yeast GAL4 repressor, and the GATA factor. Viruses also contain gene products that bind specific sequences. One of the most-studied such viral genes is the rev gene from HIV. The rev gene product binds a sequence called RRE (rev responsive element) found in the env gene. Other proteins or peptides that bind DNA may be discovered on the basis of sequence similarity to the known classes or functionally by selection.

Several techniques may be used to select other nucleic acid binding domains (see U.S. Pat. No. 5,270,170; PCT Application WO 93/14108; and U.S. Pat. No. 5,223,409). One of these techniques is phage display. (See, for example, U.S. Pat. No. 5,223,409.) In this method, DNA sequences are inserted into gene III or gene VIII gene of a filamentous phage, such as M13. Several vectors with multicloning sites have been developed (McLafferty et al., Gene 128:29-36, 1993; Scott and Smith, Science 249:386-390, 1990; Smith and Scott, Methods Enzymol. 217:228-257, 1993). The inserted DNA sequences may be randomly generated or variants of a known DNA-binding domain. Generally, the inserts encode from 6 to 20 amino acids. The peptide encoded by the inserted sequence is displayed on the surface of the bacteriophage. Bacteriophage expressing a desired nucleic acid-binding domain are selected for by binding to the cytocide-encoding agent. This target molecule may be single stranded or double stranded DNA or RNA. When the nucleic acid to be delivered is single-stranded, such as RNA, the appropriate target is single-stranded. When the molecule to be delivered is double-stranded, the target molecule is preferably double-stranded. Preferably, the entire coding region of the cytocide-encoding agent is used as the target. In addition, elements necessary for transcription that are included for in vivo or in vitro delivery may be present in the target DNA molecule. Bacteriophage that bind the target are recovered and propagated. Subsequent rounds of selection may be performed. The final selected bacteriophage are propagated and the DNA sequence of the insert is determined. Once the predicted amino acid sequence of the binding peptide is known, sufficient peptide for use herein as an nucleic acid binding domain may be made either by recombinant means or synthetically. Recombinant means is used when the receptor-binding internalized ligand/nucleic acid binding domain is produced as a fusion protein. In addition, the peptide may be generated as a tandem array of two or more peptides, in order to maximize affinity or binding of multiple DNA molecules to a single polypeptide.

As an example of the phage display selection technique, a DNA-binding domain/peptide that recognizes the coding region of saporin is isolated. Briefly, DNA fragments encoding saporin may be isolated from a plasmid containing these sequences. The plasmid FPFSI contains the entire coding region of saporin. Digestion of the plasmid with NcoI and EcoRI restriction enzymes liberates the saporin specific sequence as a single fragment of approximately 780 bp. This fragment may be purified by any one of a number of methods, such as agarose gel electrophoresis and subsequent elution from the gel. The saporin fragment is fixed to a solid support, such as in the wells of a 96-well plate. If the double-stranded fragment does not bind well to the plate, a coating such as a positively charged molecule, may be used to promote DNA adherence. The phage library is added to the wells and an incubation period allows for binding of the phage to the DNA. Unbound phage are removed by a wash, typically containing 10 mM Tris, 1 mM EDTA, and without salt or with a low salt concentration. Bound phage are eluted starting at a 0.1 M NaCl containing buffer. The NaCl concentration is increased in a step-wise fashion until all the phage are eluted. Typically, phage binding with higher affinity will only be released by higher salt concentrations. Eluted phage are propagated in the bacteria host. Further rounds of selection may be performed to select for a few phage binding with high affinity. The DNA sequence of the insert in the binding phage is then determined. In addition, peptides having a higher affinity may be isolated by making variants of the insert sequence and subjecting these variants to further rounds of selection.

III. Other Elements

A. Nuclear Translocation Signal

As used herein, a “nuclear translocation or targeting sequence” (NTS) is a sequence of amino acids in a protein that are required for translocation of the protein into a cell nucleus. Examples of NTSs are set forth in Table 1 below Comparison with known NTSs, and if necessary testing of candidate sequences, should permit those of skill in the art to readily identify other amino acid sequences that function as NTSs. The NTS may be derived from another polypeptide, or it may be derived from another region in the same polypeptide. The NTS is typically synthesized as a DNA sequence encoding the NTS and inserted appropriately into either the ligand or NABD gene sequence.

TABLE I

| | | |
|---|---|---|
| Source | Sequence* | SEQ ID NO. |

| | | |
|---|---|---|
| SV40 large T | Pro¹²⁶LysLysArgLysValGlu | 1 |
| Polyoma large T | Pro²⁷⁹ProLysLysAlaArgGluVal | 2 |
| Human c-Myc | Pro¹²⁰AlaAlaLysArgValLysLeuAsp | 3 |
| Adenovirus ElA | Lys²⁸¹ArgProArgPro | 4 |
| Yeast mat α₂ | Lys³IleProIleLys | 5 |
| c-Erb-A | A. Gly²²LysArgLysArgLysSer | 6 |
| | B. Ser¹²⁷LysArgValAlaLysArgLysLeu | 7 |
| | C. Ser¹⁸¹HisTrpLysGlnLysArgbysPhe | 8 |
| c-Myb | Pro⁵²¹LeuLeuLysLysIleLysGln | 9 |
| p53 | Pro³¹⁶GlnProLysLysLysPro | 10 |
| Nucleolin | Pro²⁷⁷GlyLysArgLysLysGluMetThrLysGlnLysGluValPro | 11 |
| HIV Tat | Gly⁴⁸ArgLysLysArgArgGlnArgArgArgAlaPro | 12 |
| FGF-1 | AsnTyrLysLysProLysLeu | 13 |
| FGF-2 | HisPheLysAspProLysArg | 14 |
| FGF-3 | AlaProArgArgArgLysLeu | 15 |
| FGF-4 | IleLysArgLeuArgArg | 16 |
| FGF-5 | GlyArgArg | |
| FGF-6 | IleLysArgGlnArgArg | 17 |
| FGF-7 | IleArgValArgArg | 18 |

*Superscript indicates position in protein

In order to deliver the nucleic acid to the nucleus, the conjugate should include an NTS. If the conjugate is designed such that the receptor-binding internalized ligand and linked nucleic acid binding domain is cleaved or dissociated in the cytoplasm, then the NTS should be included in a portion of the complex that remains bound to the nucleic acid, so that, upon internalization, the conjugate will be trafficked to the nucleus. Thus, the NTS is preferably included in the nucleic acid binding domain, but may additionally be included in the ligand. An NTS is preferred if the cytocide-encoding agent is DNA. If the cytocide-encoding agent is mRNA, an NTS may be omitted. The nuclear translocation sequence (NTS) may be a heterologous sequence or a may be derived from the selected ligand. All presently identified members of the FGF family of peptides contain an NTS (see, e.g. International Application WO 91/15229 and Table 2). A typical consensus NTS sequence contains an amino-tenninal proline or glycine followed by at least three basic residues in a array of seven to nine amino acids (see, e.g., Dang et al., J. Biol. Chem. 264:18019-18023, 1989; Dang et al., Mol. Cell. Biol. 8:4049-4058, 1988, and Table 1).

B. Cytoplasm-translocation Signal

Cytoplasm-translocation signal sequence is a sequence of amino acids in a protein that cause retention of proteins in the lumen of the endoplasmic reticulum and/or translocate proteins to the cytosol. A signal sequence in mammalian cells is KDEL (Lys-Asp-Glu-Leu) (SEQ ID NO. 19) (Munro and Pelham, Cell 48:899-907, 1987). Some modifications of this sequence have been made without loss of activity. For example, the sequences RDEL (Arg-Asp-Glu-Leu) (SEQ ID NO. 20) and KEEL (Lys-Glu-Glu-Leu) (SEQ ID NO. 21) confer efficient or partial retention, respectively, in plants (Denecke et al., EMBO. J. 11:2345-2355, 1992).

A cytoplasm-translocation signal sequence may be included in either the receptor-internalized binding ligand or the nucleic acid binding domain part or both. If cleavable linkers are used to link the ligand with the nucleic acid binding domain, the cytoplasm-translocation signal is preferably included in the nucleic acid binding domain, which will stay bound to the cytocide-encoding agent. Additionally, a cytoplasmic-translocation signal sequence may be included in the receptor-internalized binding ligand, as long as it does not interfere with receptor binding. Similarly, the signal sequence placed in the nucleic acid binding domain should not interfere with binding to the cytocide-encoding agent.

IV. Nucleic Acid Molecules for Delivery

A. Cytocide-encoding Agents

A cytocide-encoding agent is a nucleic acid molecule (e.g., DNA or RNA) that, upon internalization by a cell, and subsequent transcription (if DNA) and[/or] translation into a cytocidal agent, is cytotoxic or cytostatic to a cell, for example, inhibits cell growth by interfering with protein synthesis or disrupting the cell cycle.

Cytocides include saporin, the ricins, abrin, gelonin, and other ribosome inactivating proteins, Pseudomonas exotoxin, diphtheria toxin, angiogenin, tritin, dianthins 32 and 30, momordin, pokeweed antiviral protein, mirabilis antiviral protein, bryodin, angiogenin, shiga exotoxin, as well as other cytocides that are known to those of skill in the art. Inhibitors of cell cycle are well known.

DNA molecules that encode an enzyme that results in cell death or renders a cell susceptible to cell death upon the addition of another product are preferred. For example, saporin is an enzyme that cleaves rRNA and inhibits protein synthesis. Other enzymes that inhibit protein synthesis are especially well suited for use in the present invention. Alternatively, the product may be a ribozyme, antisense, or other nucleic acid molecule that causes cell death.

1. Ribosome Inactivating Proteins

Ribosome-inactivating proteins (RIPs), which include ricin, abrin, and saporin, are plant proteins that catalytically inactivate eukaryotic ribosomes. Ribosome-inactivating proteins inactivate ribosomes by interfering with the protein elongation step of protein synthesis. For example, the ribosome-inactivating protein saporin (also referred to as SAP) has been shown to inactivate 60S ribosomes by cleavage of the N-glycosidic bond of the adenine at position 4324 in the rat 28S ribosomal RNA (rRNA). The particular region in which A₄₃₂₄ is located in the rRNA is highly conserved among prokaryotes and eukaryotes; A₄₃₂₄ in 28S rRNA corresponds to A₂₆₆₀ in E. coli 23S rRNA. Several of the ribosome inactivating proteins also appear to interfere with protein synthesis in prokaryotes, such as E. coli.

Of ribosome-inactivating proteins, saporin is preferred as a cytocide, but other suitable ribosome inactivating proteins (RIPs) and toxins may be used. Other suitable RIPs include, but are not limited to, ricin, ricin A chain, maize ribosome inactivating protein, gelonin, diphtheria toxin, diphtheria toxin A chain, trichosanthin, tritin, pokeweed antiviral protein (PAP), mirabilis antiviral protein (MAP), Dianthins 32 and 30, abrin, monordin, bryodin, shiga (see, e g., WO 93/24620) and others (see, e.g., Barbieri et al., Cancer Surveys 1:489-520, 1982, and European patent application No. 0466 222, incorporated herein by reference, which provide lists of numerous ribosome inactivating proteins and their sources; see also U.S. Pat. No. 5,248,608 to Walsh et al.). Some ribosome inactivating proteins, such as abrin and ricin, contain two constituent chains: a cell-binding chain that mediates binding to cell surface receptors and internalization of the molecule and a chain responsible for toxicity. Single chain ribosome inactivating proteins (type I RIPS), such as the saporins, do not have a cell-binding chain. As a result, unless internalized, they are substantially less toxic to whole cells than the ribosome inactivating proteins that have two chains.

Several structurally related ribosome inactivating proteins have been isolated from seeds and leaves of the plant Saponaria officinalis (soapwort) (GB Patent 2,194,241 B; GP Patent 2,216,891; EP Patent 89306016). Saporin proteins for use in this invention have amino acid sequences found in the natural plant host Saponaria officinalis (e.g., SEQ ID NO. 22) or modified sequences, such as amino acid substitutions, deletions, insertions or additions, but that still express substantial ribosome inactivating activity. Purified preparations of saporin are frequently observed to include several molecular isoforms of the protein. It is understood that differences in amino acid sequences can occur in saporin from different species as well as between saporin molecules from individual organisms of the same species. Among these, SO-6 is the most active and abundant, representing 7% of total seed proteins. Saporin is very stable, has a high isoelectric point, does not contain carbohydrates, and is resistant to denaturing agents, such as sodium dodecyl sulfate (SDS), and a variety of proteases. The amino acid sequences of several saporin-6 isoforms from seeds are known, and there appear to be families of saporin ribosome inactivating proteins differing in few amino acid residues. Any of these saporin proteins or modified proteins that are cytotoxic may be used in the present invention.

Some of the DNA molecules provided herein encode saporin that has substantially the same amino acid sequence and ribosome inactivating activity as that of saporin-6 (SO-6), including isoforms, such as those that have heterogeneity at amino acid positions 48 and 91 (see, e.g., Maras et al., Biochem. Internat. 21:631-638, 1990, and Barra et al., Biotechnol. Appl. Biochem. 13:48-53, 1991; GB Patent 2,216,891 B and EP Patent 89306106). Other suitable saporin polypeptides include other members of the multi-gene family coding for isoforms of saporin-type ribosome inactivating proteins including SO-1 and SO-3 (Fordham-Skelton et al., Mol. Gen. Genet. 221:134-138, 1990), SO-2 (see, e.g., U.S. application Ser. No. 07/885,242; GB 2,216,891; see also Fordham-Skelton et al., Mol. Gen. Genet. 229:460-466, 1991), SO-4 (see, e.g., GB 2,194,241 B; see also Lappi et al., Biochem. Biophys. Res. Commun. 129:934-942, 1985) and SO-5 (see, e.g., GB 2,194,241 B; see also Montecucchi et al., Int. J. Peptide Protein Res. 33:263-267, 1989). Modified saporin may be produced by modifying the DNA encoding the protein (see, e.g., International PCT Application Ser. No. PCT/US93/05702, and U.S. application Ser. No. 07/901,718; see also U.S. patent application Ser. No. 07/885,242, and Italian Patent No. 1,231,914) by altering one or more amino acids or deleting or inserting one or more amino acids. Any such protein, or portion thereof, that exhibits cytotoxicity in standard in vitro or in vivo assays within at least about an order of magnitude of the saporin conjugates described herein is contemplated for use herein.

Saporin DNA sequence may use mamrnmalian-preferred codons (SEQ ID NO. 23). Preferred codon usage as exemplified in Current Protocols in Molecular Biology, infra, and Zhang et al. (Gene 105:61, 1991) for mammals, yeast, Drosophila, E. coli, and primates is established for saporin sequence.

In addition to saporin discussed above, other cytocides that inhibit protein synthesis are useful in the present invention. The gene sequences for these cytocides may be isolated by standard methods, such as PCR, probe hybridization of genomic or cDNA libraries, antibody screenings of expression libraries, or clones may be obtained from commercial or other sources. The DNA sequences of many of these cytocides are well known, including ricin A chain (GenBank Accession No. X02388); maize ribosome inactivating protein (GenBank Accession No. L26305); gelonin (GenBank Accession No. L12243; PCT Application WO 92/03155; U.S. Pat. No. 5,376,546; diphtheria toxin (GenBank Accession No. K01722); trichosanthin (GenBank Accession No. M34858); tritin (GenBank Accession No. D13795); pokeweed antiviral protein (GenBank Accession No. X78628); mirabilis antiviral protein (GenBank Accession No. D90347); dianthin 30 (GenBank Accession No. X59260); abrin (GenBank Accession No. X55667); shiga (GenBank Accession No. M19437) and Pseudomonas exotoxin (GenBank Accession Nos. K01397, M23348). When DNA sequences or amino acid sequences are known, DNA molecules encoding these proteins may be synthesized, and may contain mammalian-preferred codons.

The cytocide-encoding agent, such as saporin DNA sequence, is introduced into a plasmid in operative linkage with an appropriate promoter for expression of polypeptides in the organism. The plasmid can optionally include sequences, such as origins of replication that allow for the extrachromosomal maintenance of the saporin-containing plasmid, or can be designed to integrate into the genome of the host (as an alternative means to ensure stable maintenance in the host).

2. Other Cytocide-encoding Agents

The conjugates provided herein may also be used to deliver a ribozyme, antisense, and the like to targeted cells. These nucleic acids may be present in the complex of ligand and nucleic acid binding domain or encoded by a nucleic acid in the complex. Alternatively, the nucleic acid may be directly linked to the ligand. Such products include antisense RNA, antisense DNA, ribozymes, triplex-forming oligonucleotides, and oligonucleotides that bind proteins. The nucleic acids can also include RNA trafficking signals, such as viral packaging sequences (see, e.g.. Sullenger et al. (1994) Science 262:1566-1569).

Nucleic acids and oligonucleotides for use as described herein can be synthesized by any method known to those of skill in this art (see, e.g., WO 93/01286, U.S. application Ser. No. 07/723,454; U.S. Pat. No. 5,218,088; U.S. Pat. No. 5,175,269; U.S. Pat. No. 5,109,124). Identification of oligonucleotides and ribozymes for use as antisense agents and DNA encoding genes for targeted delivery for genetic therapy involve methods well known in the art. For example, the desirable properties, lengths and other characteristics of such oligonucleotides are well known. Antisense oligonucleotides are typically designed to resist degradation by endogenous nucleolytic enzymes by using such linkages as: phosphorothioate, methylphosphonate, sulfone, sulfate, ketyl, phosphorodithioate, phosphoramidate, phosphate esters, and other such linkages (see, e.g., Agrwal et al., Tetrehedron Lett. 28:3539-3542 (1987); Miller et al., J. Am. Chem. Soc. 93:6657-6665 (1971); Stec et al., Tetrehedron Lett. 26:2191-2194 (1985); Moody et al., Nucl. Acids Res. 12:4769-4782 (1989); Uznanski et al., Nucl. Acids Res. (1989); Letsinger et al., Tetrahedron 40:137-143 (1984); Eckstein, Annu. Rev. Biochem. 54:367-402 (1985); Eckstein, Trends Biol. Sci. 14:97-100 (1989); Stein In: Oligodeoxynucleotides. Antisense Inhibitors of Gene Expression, Cohen, Ed, Macmillan Press, London, pp. 97-117 (1989); Jager et al., Biochemistry 27:7237-7246 (1988)).

Antisense nucleotides are oligonucleotides that bind in a sequence-specific manner to nucleic acids, such as mRNA or DNA. When bound to mRNA that has complementary sequences, antisense prevents translation of the mRNA (see, e.g., U.S. Pat. No. 5,168,053 to Altman et al.; U.S. Pat. No. 5,190,931 to Inouye, U.S. Pat. No. 5,135,917 to Burch; U.S. Pat. No. 5,087,617 to Smith and Clusel et al. (1993) Nucl. Acids Res. 21:3405-3411, which describes dumbbell antisense oligonucleotides). Triplex molecules refer to single DNA strands that bind duplex DNA forming a colinear triplex molecule, thereby preventing transcription (see, e.g., U.S. Pat. No. 5,176,996 to Hogan et al., which describes methods for making synthetic oligonucleotides that bind to target sites on duplex DNA).

Particularly useful antisense nucleotides and triplex molecules are molecules that are complementary or bind to the sense strand of DNA or mRNA that encodes a protein involved in cell proliferation, such as an oncogene or growth factor, (e.g., bFGF, int-2, hst-1/K-FGF, FGF-5, hst-2/FGF-6, FGF-8). Other useful antisense oligonucleotides include those that are specific for IL-8 (see, e.g., U.S. Pat. No. 5,241,049), c-src, c-fos H-ras (lung cancer), K-ras (breast cancer), urokinase (melanoma), BCL2 (T-cell lymphoma), IGF-1 (glioblastoma), IGF-1 receptor (glioblastoma), TGF-β1, and CRIPTO EGF receptor (colon cancer). These particular antisense plasmids reduce tumorigenicity in athymic and syngeneic mice.

These nucleic acids or nucleic acids that encode antisense can be linked to bFGF for the treatment of psoriasis. Anti-sense oligonucleotides or nucleic acids encoding antisense specific for nonmuscle myosin heavy chain and/or c-myb (see, e.g., Simons et al. (1992) Circ. Res. 70:835-843; PCT Application WO 93/01286, U.S. application Ser. No. 07/723,454: LeClerc et al. (1991) J. Am. Coll. Cardiol. 17 (2Suppl. A):105A; Ebbecke et al. (1992) Basic Res. Cardiol. 87:585-591) can be targeted by an FGF, for example to inhibit smooth muscle cell proliferation, such as occurs following angioplasty.

A ribozyme is an RNA molecule that specifically cleaves RNA substrates, such as MRNA, resulting in inhibition or interference with cell growth or expression. There are at least five known classes of ribozymes involved in the cleavage and/or ligation of RNA chains. Ribozymes can be targeted to any RNA transcript and can catalytically cleave such transcript (see, e.g., U.S. Pat. No. 5,272,262; U.S. Pat. No. 5,144,019; and U.S. Pat. Nos. 5,168,053, 5,180,818, 5,116,742 and 5,093,246 to Cech et al.). Any such ribozyme or nucleic acid encoding the ribozyme may be delivered to a cell bearing a receptor for a receptor-internalized binding ligand.

Ribozymes and the like may be delivered to the targeted cells by DNA encoding the ribozyme linked to a eukaryotic promoter, such as an eukaryotic viral promoter, such that upon introduction into the nucleus, the ribozyme will be directly transcribed. In such instances, the construct will also include a nuclear translocation sequence, generally as part of the ligand or as part of a linker between the ligand and nucleic acid binding domain.

B. Prodrug-encoding Agent

A nucleic acid molecule encoding a prodrug may alternatively be used within the context of the present invention. Prodrugs are inactive in the host cell until either a substrate or an activating molecule is provided. Most typically, a prodrug activates a compound with little or no cytotoxicity into a toxic compound. Two of the more often used prodrug molecules, both of which are suitable for use in the present invention, are HSV thyrnidine kinase and E. coli cytosine deaminase.

Briefly, a wide variety of gene products which either directly or indirectly activate a compound with little or no cytotoxicity into a toxic product may be utilized within the context of the present invention. Representative examples of such gene products include HSVTK (herpes simplex virus thymidine kinase) and VZVTK (varicella zoster virus thymidine kinase), which selectively phosphorylate certain purine arabinosides and substituted pyrimidine compounds. Phosphorylation converts these compounds to metabolites that are cytotoxic or cytostatic. For example, exposure of the drugs ganciclovir, acyclovir, or any of their analogues (e.g., FIAU, FIAC, DHPG) to cells expressing HSVTK allows conversion of the drug into its corresponding active nucleotide triphosphate form.

Other gene products that may be utilized within the context of the present invention include E. coli guanine phosphoribosyl transferase, which converts thioxanthine into toxic thioxanthine monophosphate (Besnard et al., Mol. Cell. Biol. 7:4139-4141, 1987); alkaline phosphatase, which converts inactive phosphorylated compounds such as mitomycin phosphate and doxorubicin-phosphate to toxic dephosphorylated compounds; fimgat (e.g., Fusarium oxysporum) or bacterial cytosine deaminase, which converts 5-fluorocytosine to the toxic compound 5-fluorouracil (Mullen, PNAS 89:33, 1992); carboxypeptidase G2, which cleaves glutamic acid from para-N-bis (2-chloroethyl) aminobenzoyl glutamic acid, thereby creating a toxic benzoic acid mustard; and Penicillin-V amidase, which converts phenoxyacetabide derivatives of doxorubicin and melphalan to toxic compounds (see generally, Vrudhula et al., J. Med. Chem. 36:919-923, 1993; Kern et al., Canc. Immun. Immunother. 31:202-206, 1990). Moreover, a wide variety of Herpesviridae thymidine kinases, including both primate and non-primate herpesviruses, are suitable. Such herpesviruses include Herpes Simplex Virus Type I (McKnight et al., Nuc. Acids Res 8:5949-5964, 1980), Herpes Simplex Virus Type 2 (Swain and Galloway, J. Virol. 46:1045-1050, 1983), Varicella Zoster Virus (Davison and Scott, J. Gen. Virol. 67:1759-1816, 1986), marmoset herpesvirus (Otsuka and Kit, Virology 135:316-330, 1984), feline herpesvirus type 1 (Nunberg et al., J. Virol. 63:3240-3249, 1989), pseudorabies virus (Kit and Kit, U.S. Pat. No. 4,514,497, 1985), equine herpesvirus type I (Robertson and Whalley, Nuc. Acids Res. 16:11303-11317, 1988), bovine herpesvirus type I (Mittal and Field, J. Virol 70:2901-2918, 1989), turkey herpesvirus (Martin et al., J. Virol. 63:2847-2852, 1989), Marek's disease virus (Scott et al., J. Gen. Virol. 70:3055-3065, 1989), herpesvirus saimiri (Honess et al., J. Gen. Virol. 70:3003-3013, 1989) and Epstein-Barr virus (Baer et al., Nature (London) 310:207-311, 1984). Such herpesviruses may be readily obtained from commercial sources such as the American Type Culture Collection (“ATCC”, Rockville, Md.).

Furthermore, as indicated above, a wide variety of inactive precursors may be converted into active inhibitors. For example, thymidine kinase can phosphorylate nucleosides (e.g., dT) and nucleoside analogues such as ganciclovir (9-{[2-hydroxy-1-(hydroxymethyl)ethoxyl methyl} guanosine), famciclovir, buciclovir, penciclovir, valciclovir, acyclovir (9-[2-hydroxy ethoxy)methyl] guanosine), trifluorothymidine, 1-[2-deoxy, 2-fluoro, beta-D-arabino furanosyl]-5-iodouracil, ara-A (adenosine arabinoside, vivarabine), 1-beta-D-arabinofuranoxyl thymine, 5-ethyl-2′-deoxyuridine, 5-iodo-5′-amino-2,5′-dideoxyuridine, idoxuridine (5-iodo-2′-deoxyuridine), AZT (3′ azido-3′ thymidine), ddC (dideoxycytidine), AIU (5-iodo-5′ amino 2′, 5′-dideoxyurdine) and AraC (cytidine arabinoside).

Other gene products may render a cell susceptible to toxic agents. Such products include tumor necrosis factor, viral proteins, and channel proteins that transport drugs.

A cytocide-encoding agent may be constructed as a prodrug, which when expressed in the proper cell type is processed or modified to an active form. For example, the saporin gene may be constructed with an N- or C-terminal extension containing a protease-sensitive site. The extension renders the protein inactive and subsequent cleavage in a cell expressing the appropriate protease restores enzymatic activity.

C. Cytokine Immunotherapy

Cytokine immunotherapy is a modification of immunogene therapy and involves the administration of tumor cell vaccines that are genetically modified ex viva or in vivo to express various cytokine genes. In animal tumor models, cytokine gene transfer resulted in significant antitumor immune response (Fearon, et al., Cell 60: 387-403, 1990; Wantanabe, et al., Proc. Nat. Acad. Sci USA, 86: 9456-9460, 1989).

In the present invention, the ligand-NABD conjugate is used to deliver DNA encoding a cytokine, such as IL-12, IL-10, IL-2, GM-CSF, INF-γ, or an MHC gene, such as HLA-B7. Delivery of these genes will modulate the immune system, increasing the potential for host antitumor immunity. Alternatively, DNA encoding costimulatory molecules, such as B7.1 and B7.2, ligands for CD28 and CTLA-4 respectively, can also be delivered to enhance T cell mediated immunity. These genes can be co-delivered with cytokine genes, using the same or different promoters and optionally with an internal ribosome binding site. Similiarly, α-1,3-galactosyl transferase expression on tumor cells allows complement-mediated cell killing.

D. Construct Containing a Nucleic Acid for Delivery

In the case of cytocide molecules such as the ribosome inactivating proteins, very few molecules may need to be expressed to effect cell killing. Indeed, only a single molecule of diphtheria toxoid introduced into a cell is sufficient to kill the cell. With other cytocides or prodrugs, it may be that propagation or stable maintenance of the construct is necessary to attain a sufficient amount or concentration of the gene product for effective gene therapy. Examples of replicating and stable eukaryotic plasmids may be found in the scientific literature.

In general, constructs will also contain elements necessary for transcription and translation. If the cytocide-encoding agent is DNA, then it must contain a promoter. The choice of the promoter will depend upon the cell type to be transformed and the degree or type of control desired. Promoters can be constitutive or active in any cell type, tissue specific, cell specific, event specific, temporal-specific or inducible. Cell-type specific promoters and event type specific promoters are preferred. Examples of constitutive or nonspecific promoters include the SV40 early promoter (U.S. Pat. No. 5,118,627), the SV40 late promoter (U.S. Pat. No. 5,118,627), CMV early gene promoter (U.S. Pat. No. 5,168,062), and adenovirus promoter. In addition to viral promoters, cellular promoters are also amenable within the context of this invention. In particular, cellular promoters for the so-called housekeeping genes are useful. Viral promoters are preferred, because generally they are stronger promoters than cellular promoters.

Tissue specific promoters are particularly useful for expression in tumor cells. By using one of this class of promoters, an extra margin of specificity can be attained. Such promoters include promoters for prostate specific antigen-1, probasin (mainly for prostate tumor), FGF receptor (e.g. leukernias, gliomas, sarcomas), VEGF receptor (mainly for angiogenic tumors, gliomas, colon carcinoma, hemangioblastomas), erb B2; erb B3; erb B4; MUC-1; HSP-27; int-1; int-2 (mainly for breast tumors), CEA (mainly for colon, lung, pancreatic, gastric, breast, and ovarian tumors), HBEGF receptor; EGF receptor (mainly for bladder tumors); tyrosinase (melanoma), MAGE (mainly for melanomas), IL-2, IL-2 receptor (mainly for T cell tumors), alpha-fetoprotein (mainly for hepatocellular carcinomas, non-seminomatous testicular cancer, some gastrointestinal tumors), prostatic acid phosphatase (prostate tumor), probasin (prostate tumor), prostate specific membrane antigen (prostate tumor), alpha-crystallin (lens cells), and tie-2 (angiogenic tumors).

Such promoters also include promoters active in angiogenesis (eg., VEGF-receptor promoter (Morishita et al., J. Biol. Chem. 270:27948, 1995; GenBank Accession No. X89776); FGF receptor promoter; TEK or tie 2 promoter, (Huang et al., Oncogene 11:2097, 1995; GenBank Accession No. L06139); tie (WO 96/09381; Korhonen et al., Blood 86:1828, 1995; GenBank Accession No. X60954; GenBankAccession No. S89716); urokinase receptor, (Hollas et al., Cancer Res. 51:3690, 1991; Gum et al., Anti-Cancer Res. 15:1167, 1995; Soravia et al., Blood 86:624, 1995; GenBank Accession No. S78532); E- and P-selectin, (Fox et al., J. Pathol. 177:369, 1995; Biancone et al., J. Exp. Med. 183:581, 1996; GenBank Accession No. M64485; L01874); VCAM-1 (Iademarco et al., J. Biol. Chem. 267:16323, 1992; GenBank Accession No. M92431); endoglin (Bellon et al., Eur. J. Immunol. 23:2340, 1993; Gougos and Letarte, J. Biol. Chem. 265:8361, 1990; GenBank Accession No. HSENDOG); endosialin (Rettig et al., PNAS 89:10832, 1992); alpha V-beta3 integrin (Villa-Garcia et al., Blood 3:668, 1994; Donahue et al., BBA 1219:228, 1994); endothelin-1 (GenBank Accession No. M25377; GenBank Accession No. J04819, J05489); ICAM-3 (Patey et al., Am. J. Pathol. 148:465, 1996; Fox et al., J. Path. 177:369, 1995; GenBank Accession No. S50015); E9 antigen (Wang et al., Int. J. Cancer 54:363, 1993); von Willebrand factor (Jahroudi and Lynch, Mol. Cell. Biol. 14:999, 1994; GenBank Accession No. HUMVWFI; HUMVWFA); CD44 (Hofnann et al., Cancer Res. 53:1516, 1993; Maltzman et al., Mol. Cell. Biol. 16:2283, 1996; GenBank Accession No. HUMCD44B); CD40 (Pammer et al., Am. J. Pathol. 148:13 87, 1996; GenBank Accession No. HACD40L; GenBank Accession No. HSCD405FR); vascular-endothelial cadherin (Martin-Padura et al., J. Pathol. 175:51, 1995); notch 4 (Uyttendaele et al., Development 122:2251, 1996) and high molecular weight melanoma-associated antigen).

Other tumor-specific promoters may be identified by a differential display method (see, U.S. Pat. No. 5,262,311. Briefly, in this method, genes expressed to a higher level than in normal counterpart cells are identified after amplification. The gene is cloned. A genomic gene isolated and the promoter obtained from a genomic clone.

Inducible promoters may also be used. These promoters include MMTV LTR (PCT WO 91/13160), inducible by dexarnethasone, metallothionein, inducible by heavy metals, erg-1 inducible with radiation, and promoters with cAMP response elements, inducible by cAMP. By using an inducible promoter, the nucleic acid may be delivered to a cell and will remain quiescent until the addition of the inducer. This allows further control on the timing of production of the gene product.

Event-type specific promoters are active or up-regulated only upon the occurrence of an event, such as tumorigenecity or viral infection. The HIV LTR is a well known example of an event-specific promoter. The promoter is inactive unless the tat gene product is present, which occurs upon viral infection. Some event-type promoters are also tissue-specific.

Additionally, promoters that are coordinately regulated with a particular cellular gene may be used. For example, promoters of genes that are coordinately expressed when a particular FGF receptor gene is expressed may be used. Then, the nucleic acid will be transcribed when the FGF receptor, such as FGFR1, is expressed, and not when FGFR2 is expressed. This type of promoter is especially useful when one knows the pattern of FGF receptor expression in a particular tissue, so that specific cells within that tissue may be killed upon transcription of a cytotoxic agent gene without affecting the surrounding tissues.

If the domain binds in a sequence specific manner, the construct must contain the sequence that binds to the nucleic acid binding domain. As described below, the target nucleotide sequence may be contained within the coding region of the cytocide, in which case, no additional sequence need be incorporated. Additionally, it may be desirable to have multiple copies of target sequence. If the target sequence is coding sequence, the additional copies must be located in non-coding regions of the cytocide-encoding agent. The target sequences of the nucleic acid binding domains are typically generally known. If unknown, the target sequence may be readily determined. Techniques are generally available for establishing the target sequence (e.g., see PCT Application WO 92/05285 and U.S. Ser. No 586,769).

In addition to the promoter, repressor sequences, negative regulators, or tissue-specific silencers may be inserted to reduce non-specific expression of the cytocide or prodrug. Multiple repressor elements may be inserted in the promoter region. Repression of transcription is independent on the orientation of repressor elements or distance from the promoter. One type of repressor sequence is an insulator sequence. Such sequences inhibit transcription (Dunaway et al., Mol Cell Biol 17: 182-9, 1997; Gdula et al., Proc Natl Acad Sci USA 93:9378-83, 1996, Chan et al., J Virol 70: 5312-28, 1996; Scott and Geyer, EMBO J 14: 6258-67, 1995; Kalos and Fournier, Mol Cell Biol 15: 198-207, 1995; Chung et al., Cell 74: 505-14, 1993) and will silence background transcription.

Negative regulatory elements have been characterized in the promoter regions of a number of different genes. The repressor element functions as a repressor of transcription in the absence of factors, such as steroids, as does the NSE in the promoter region of the ovalbumin gene (Haecker et al., Mol. Endocrinology 9:1113-1126, 1995). These negative regulatory elements bind specific protein complexes from oviduct, none of which are sensitive to steroids. Three different elements are located in the promoter of the ovalbumin gene. Oligonucleotides corresponding to portions of these elements repress viral transcription of the TK reporter. One of the silencer elements shares sequence identity with silencers in other genes (TCTCTCCNA).

Repressor elements have also been identified in the promoter region of collagen II gene. Gel retardation studies showed that nuclear factors from HeLa cells bind specifically to DNA fragments containing the silencer region, whereas chrondocyte nuclear extracts did not show any binding activity (Savanger et al., J. Biol. Chem. 265(12):6669-6674, 1990). Repressor elements have also been shown to regulate transcription in the carbamyl phosphate synthetase gene (Goping et al., Nucleic Acid Research 23(10):1717-1721, 1995). This gene is expressed in only two different cell types, hepatocytes and epithelial cells of the intestinal mucosa. Negative regulatory regions have also been identified in the promoter region of the choline acetyltransferase gene, the albumin promoter (Hu et al., J. Cell Growth Differ. 3(9):577-588, 1992), phosphoglycerate kinase (PGK-2) gene promoter (Misuno et al., Gene 119(2):293-297, 1992), and in the 6-phosphofructo-2-kinase/fructose-2, 6-bisphosphatase gene, in which the negative regulatory element inhibits transcription in non-hepatic cell lines (Lemaigre et al., Mol. Cell Biol. 11(2):1099-1106). Furthermore, the negative regulatory element Tse-1 has been identified in a number of liver specific genes. including tyrosine aminotransferase (TAT). TAT gene expression is liver specific and inducible by both glucocorticoids and the cAMP signaling pathway. The cAMP response element (CRE) has been shown to be the target for repression by Tse-1 and hepatocyte-specific elements (Boshart et al., Cell 61(5):905-916, 1990).

In preferred embodiments, elements that increase the expression of the desired product are incorporated into the construct. Such elements include internal ribosome binding sites (IRES; Wang and Siddiqui, Curr. Top. Microbiol. Immunol 203:99, 1995; Ehrenfeld and Semler, Curr. Top. Microbiol. Immunol. 203:65, 1995; Rees, et al., Biotechniques 20:102, 1996; Sugimoto et al., Biotechnology 12:694, 1994). IRES increase translation efficiency. As well, other sequences may enhance expression. For some genes, sequences especially at the 5′ end inhibit transcription and/or translation. These sequences are usually palindromes that can form hairpin structures. Any such sequences in the nucleic acid to be delivered are generally deleted. Expression levels of the transcript or translated product are assayed to confirm or ascertain which sequences affect expression. Transcript levels may be assayed by any known method, including Northern blot hybridization, RNase probe protection and the like. Protein levels may be assayed by any known method, including ELISA.

Other elements may be incorporated into the construct. In preferred embodiments, the construct includes a transcription terminator sequence, including a polyadenylation sequence, splice donor and acceptor sites, and an enhancer. Other elements useful for expression and maintenance of the construct in mammalian cells or other eukaryotic cells may also be incorporated (e.g., origin of replication). Because the constructs are conveniently produced in bacterial cells, elements that are necessary or enhance propagation in bacteria are incorporated. Such elements include an origin of replication, selectable marker and the like (see discussion below).

An additional level of control for initiating expression of the nucleic acid only in appropriate cells or enhancing uptake of complex is the delivery of two constructs, one of which encodes the cytocide and the other construct encodes a second gene that controls expression of the promoter driving the cytocide or prodrug or enhances uptake of the complexes into tumor masses or other target cells. By way of example, on one construct, the cytocide encoding agent is controlled by a promoter, such as a heat shock promoter. The second construct is a gene, such as a gene that elicits SOS pathway under control of a tumor-specific promoter. The two constructs are co-delivered or sequentially delivered. When delivered into tumor cells, the SOS gene is expressed and results in causing expression of the cytocide-encoding agent. In this case, the two constructs could be merged into one construct.

In the other type of multiple delivery system, the first construct is a cytocide gene under control of a promoter, such as those described above. The second construct comprises a different promoter controlling expression of a gene, such as IL-2, that induces leakiness in a tumor mass to allow better penetration. When the second construct is introduced first, the tumor mass will be more readily accessible for the first construct to be delivered.

Typically, the constructs are plasmid vectors. A preferred construct is a modified pNASS vector (Clontech, Palo Alto, Calif.). In the modified vector, amp resistance gene is replaced by kan resistance gene, a poly A signal sequence is added upstream of the mammalian promoter. A T7 promoter is added downstream of the mammalian promoter and upstream of the cytocide or prodrug gene to facilitate verification of cytotoxic activity. Other suitable mammalian expression vectors are well known (see, e.g., Ausubel et al., 1995; Sambrook et al., supra; Invitrogen catalogue, San Diego, Calif.; Novagen, Madison, Wis.; Pharmacia catalogue, Uppsala, Sweden; and others).

V. Expression Vectors and Host Cells for Expression of Ligands and NABD

Host organisms include those organisms in which recombinant production of heterologous proteins have been carried out, such as bacteria (for example, E. coli), yeast (for example, Saccharomyces cerevisiae and Pichia pastoris), mammalian cells, and insect cells. Presently preferred host organisms are E. coli bacterial strains.

The DNA construct encoding the desired protein is introduced into a plasmid for expression in an appropriate host. In preferred embodiments, the host is a bacterial host. The sequence encoding the ligand or nucleic acid binding domain is preferably codon-optimized for expression in the particular host. Thus, for example, if human FGF-2 is expressed in bacteria, the codons would be optimized for bacterial usage. For small coding regions, the gene can be synthesized as a single oligonucleotide. For larger proteins, splicing of multiple oligonucleotides, mutagenesis, or other techniques known to those in the art may be used. For example, the sequence of a bacterial-codon preferred FGF-SAP fusion is shown in SEQ ID NO. 24. The sequences of nucleotides in the plasmids that are regulatory regions, such as promoters and operators, are operationally associated with one another for transcription. The sequence of nucleotides encoding the growth factor or growth factor-chimera may also include DNA encoding a secretion signal, whereby the resulting peptide is a precursor protein. The resulting processed protein may be recovered from the periplasmic space or the fermentation medium.

In preferred embodiments, the DNA plasmids also include a transcription terminator sequence. As used herein, a “transcription terminator region” is a sequence that signals transcription termination. The entire transcription terminator may be obtained from a protein-encoding gene, which may be the same or different from the inserted gene or the source of the promoter Transcription terminators are optional components of the expression systems herein, but are employed in preferred embodiments.

The plasmids used herein include a promoter in operative association with the DNA encoding the protein or polypeptide of interest and are designed for expression of proteins in a bacterial host. It has been found that tightly regulatable promoters are preferred for expression of saporin. Suitable promoters for expression of proteins and polypeptides herein are widely available and are well known in the art. Inducible promoters or constitutive promoters that are linked to regulatory regions are preferred. Such promoters include, but are not limited to, the T7 phage promoter and other T7-like phage promoters, such as the T3, T5 and SP6 promoters, the trp, Ipp, and lac promoters, such as the lacUV5, from E. coli; the P10 or polyhedron gene promoter of baculovirus/insect cell expression systems (see, e.g., U.S. Pat. Nos. 5,243,041, 5,242,687, 5,266,317, 4,745,051, and 5,169,784) and inducible promoters from other eukaryotic expression systems. For expression of the proteins such promoters are inserted in a plasmid in operative linkage with a control region such as the lac operon

Preferred promoter regions are those that are inducible and functional in E. coli. Examples of suitable inducible promoters and promoter regions include, but are not limited to: the E. coli lac operator responsive to isopropyl β -D-thiogalactopyranoside (IPTG; see, et al. Nakamura et al., Cell 18:1109-1117, 1979); the metallothionein promoter metal-regulatory-elements responsive to heavy-metal (e.g., zinc) induction (see, e.g., U.S. Pat. No. 4,870,009 to Evans et al.); the phage T7lac promoter responsive to IPTG (see, e.g., U.S. Pat. No. 4,952,496; and Studier et al., Meth Enzymol. 185:60-89,1990) and the TAC promoter.

The plasmids also preferably include a selectable marker gene or genes that are fimctional in the host. A selectable marker gene includes any gene that confers a phenotype on bacteria that allows transformed bacterial cells to be identified and selectively grown from among a vast majority of untransformed cells. Suitable selectable marker genes for bacterial hosts, for example, include the ampicillin resistance gene (Amp^{r}), tetracycline resistance gene (Tc^{r}) and the kanamycin resistance gene (Kan^{r}). The kanamycin resistance gene is presently preferred.

The plasmids may also include DNA encoding a signal for secretion of the operably linked protein. Secretion signals suitable for use are widely available and are well known in the art. Prokaryotic and eukaryotic secretion signals functional in E. coli may be employed. The presently preferred secretion signals include, but are not limited to, those encoded by the following E. coli genes: ompA, ompT, ompF, ompC, beta-lactamase, and alkaline phosphatase, and the like (von Heijne, J. Mol. Biol. 184:99-105, 1985). In addition, the bacterial pelB gene secretion signal (Lei et al., J. Bacteriol. 169:4379, 1987), the phoA secretion signal, and the cek2 functional in insect cell may be employed. The most preferred secretion signal is the E. coli ompA secretion signal. Other prokaryotic and eukaryotic secretion signals known to those of skill in the art may also be employed (see, e.g., von Heijne, J. Mol. Biol. 184:99-105, 1985). Using the methods described herein, one of skill in the art can substitute secretion signals that are functional in either yeast, insect or mammalian cells to secrete proteins from those cells.

Particularly preferred plasmids for transformation of E. coli cells include the pET expression vectors (see U.S. Pat. No. 4,952,496; available from Novagen, Madison, Wis.; see also literature published by Novagen describing the system). Such plasmids include pET 11a, which contains the T71ac promoter, T7 terminator, the inducible E. coli lac operator, and the lac repressor gene; pET 12a-c, which contains the T7 promoter, T7 terminator, and the E. coli ompT secretion signal; and pET 15b (Novagen, Madison, Wis.), which contains a His-Tag™ leader sequence for use in purification with a His column and a thrombin cleavage site that permits cleavage following purification over the column, the T7-lac promoter region and the T7 terminator.

Other preferred plasmids include the pKK plasmids, particularly pKK 223-3, which contains the tac promoter, (available from Pharmacia; see also Brosius et al., Proc. Natl. Acad. Sci. 81:6929, 1984; Ausubel et al., Current Protocols in Molecular Biology; U.S. Pat. Nos. 5,122,463, 5,173,403, 5,187,153, 5,204,254, 5,212,058, 5,212,286, 5,215,907, 5,220,013, 5,223,483, and 5,229,279). Plasmid pKK has been modified by replacement of the ampicillin resistance marker gene, by digestion with EcoRI, with a kanamycin resistance cassette with EcoRI sticky ends (purchased from Pharmacia; obtained from pUC4K, see, e.g., Vieira et al. (Gene 19:259-268, 1982; and U.S. Pat. No.4,719,179). Baculovirus vectors, such as pBlueBac (also called pJVETL and derivatives thereof), particularly pBlueBac III, (see, e.g., U.S. Pat. Nos. 5,278,050, 5,244,805, 5,243,041, 5,242,687, 5,266,317, 4,745,051, and 5,169,784; available from Invitrogen, San Diego) may also be used for expression of the polypeptides in insect cells. The pBlueBacIII vector is a dual promoter vector and provides for the selection of recombinants by blue/white screening as this plasmid contains the β-galactosidase gene (lacZ) under the control of the insect recognizable ETL promoter and is inducible with IPTG. A DNA construct may be made in baculovirus vector pBluebac III and then co-transfected with wild type virus into insect cells Spodoptera frugiperda (sf9 cells; see, e.g., Luckow et al., Bio/technology 6:47-55, 1988, and U.S. Pat. No. 4,745,051).

Other plasmids include the pIN-IllompA plasmids (see U.S. Pat. No. 4,575,013; see also Duffaud et al., Meth Enz. 153:492-507, 1987), such as pIN-IIIompA2. The pIN-IlIompA plasmids include an insertion site for heterologous DNA linked in transcriptional reading frame with four functional fragments derived from the lipoprotein gene of E. coli. The plasmids also include a DNA fragment coding for the signal peptide of the ompA protein of E. coli, positioned such that the desired polypeptide is expressed with the ompA signal peptide at its amino terminus, thereby allowing efficient secretion across the cytoplasmic membrane. The plasmids further include DNA encoding a specific segment of the E. coli lac promoter-operator, which is positioned in the proper orientation for transcriptional expression of the desired polypeptide, as well as a separate functional E. coli lacI gene encoding the associated repressor molecule that, in the absence of lac operon inducer, interacts with the lac promoter-operator to prevent transcription therefrom. Expression of the desired polypeptide is under the control of the lipoprotein (lpp) promoter and the lac promoter-operator, although transcription from either promoter is normally blocked by the repressor molecule. The repressor is selectively inactivated by means of an inducer molecule thereby inducing transcriptional expression of the desired polypeptide from both promoters.

Preferably, the DNA fragment is replicated in bacterial cells, preferably in E. coli. The preferred DNA fragment also includes a bacterial origin of replication, to ensure the maintenance of the DNA fragment from generation to generation of the bacteria. In this way, large quantities of the DNA fragment can be produced by replication in bacteria. Preferred bacterial origins of replication include, but are not limited to, the fl-ori and col E1 origins of replication. Preferred hosts contain chromosomal copies of DNA encoding T7 RNA polymerase operably linked to an inducible promoter, such as the lacUV promoter (see U.S. Pat. No. 4,952,496). Such hosts include, but are not limited to, lysogens E. coli strains HMS174(DE3)pLysS, BL21(DE3)pLysS, HMS174(DE3) and BL21(DE3). Strain BL21(DE3) is preferred. The pLys strains provide low levels of T7 Iysozyme, a natural inhibitor of T7 RNA polymerase.

The DNA fragments provided may also contain a gene coding for a repressor protein. The repressor protein is capable of repressing the transcription of a promoter that contains sequences of nucleotides to which the repressor protein binds. The promoter can be derepressed by altering the physiological conditions of the cell. For example, the alteration can be accomplished by adding to the growth medium a molecule that inhibits the ability to interact with the operator or with regulatory proteins or other regions of the DNA or by altering the temperature of the growth media. Preferred repressor proteins include, but are not limited to the E. coli lacI repressor responsive to IPTG induction, the temperature sensitive λ cI57 repressor, and the like. The E. coli lad repressor is preferred.

By way of example, DNA encoding FGF-2 or an FGF-2 mutein is linked to DNA encoding an nucleic acid binding domain, such as protamine, and introduced into a pET expression vector, such as pET-11a and pET-12a (Novagen, Madison, Wis.), for intracellular and periplasmic expression, respectively, of the fusion protein.

VI. Preparation of Ligand-directed Nucleic Acid Delivery Vehicles

Within the context of this invention, specificity of delivery in a cell-specific manner is achieved through the ligand. The choice of the receptor-binding internalized ligand to use will depend upon the receptor expressed by the target cells. The receptor type of the target cell population may be determined by conventional techniques such as antibody staining, PCR of cDNA using receptor-specific primers, and biochemical or functional receptor binding assays. It is preferable that the receptor be cell type-specific or have increased expression or activity (i e., higher rate of internalization) within the target cell population. Typically, a nucleic acid binding domain is coupled to a receptor-binding internalized ligand, either by chemical conjugation or as a fusion protein. As described below, the ligand may alternatively be coupled directly to the nucleic acid and then complexed with a nucleic acid binding protein, such as poly-lysine (either the D- or L-form), which serves to condense the nucleic acid. Linkers as described above may optionally be used.

The complexes are tested in vitro and in vivo for the desired effect. Thus, if the nucleic acid encodes a cytocide, cell cytotoxicity or inhibition of protein synthesis or other function is measured. Cell death is conveniently assayed by counting the number of living cells in the presence and absence of delivery. Other assays, such as MTS, ³H-leu uptake, ³H-thymidine incorporation, flow cytometry, or staining cells with viable dyes are also suitable.

A. Preparation of Ligand-NABD Conjugates

1. Fusion Protein of Ligands and Nucleic Acid Binding Domain

As a preferred alternative, the conjugate is a fusion protein of receptor-binding internalized ligand, optional linker, and nucleic acid binding domain. A fusion protein is a more homogeneous preparation. As well, aggregate formation can be reduced in preparations containing the fusion proteins by modifying the receptor-binding internalized ligand, such as by removal of nonessential cysteines, and/or the nucleic acid binding domain to prevent interactions between conjugates via free cysteines. Optionally, one or more coding regions for an endosome-disruptive peptide or cytoplasmic retention signal may be constructed as part of the fusion protein.

DNA encoding the polypeptides may be isolated, synthesized or obtained from commercial sources or prepared as described herein. Expression of recombinant polypeptides may be performed as described herein or by other well known methods; and DNA encoding these polypeptides may be used as the starting materials for the methods herein.

As described above, DNA encoding a ligand is prepared synthetically based on the amino acid or DNA sequence or may be isolated using methods known to those of skill in the art, such as PCR, probe hybridization of libraries, and the like or obtained from commercial or other sources. For example, suitable methods are described in the Examples for amplifying FGF encoding cDNA from plasmids containing FGF encoding cDNA.

As described herein, such DNA can then be mutagenized using standard methodologies to delete or replace any cysteine residues that are responsible for aggregate formation. If necessary, the identity of cysteine residues that contribute to aggregate formation may be determined empirically, by deleting and/or replacing a cysteine residue and ascertaining whether the resulting growth factor with the deleted cysteine forms aggregates in solutions containing physiologically acceptable buffers and salts. Loci for insertion of cysteine residues may also be determined empirically. Generally, regions at or near (within 20, preferably 10 amino acids) the C- or, preferably, the N-terminus are preferred.

The DNA construct encoding the fusion protein can be inserted into a plasmid and expressed in a selected host, as described above, to produce a recombinant receptor-binding internalized ligand—nucleic acid binding domain conjugate. Multiple copies of the chimera can be inserted into a single plasmid in operative linkage with one promoter. When expressed, the resulting protein will then be a multimer. Typically, two to six copies of the chimera are inserted, preferably in a head to tail fashion, into one plasmid.

Removal of cysteines not required for binding and internalization is preferred for both chemical conjugation and recombinant methods. Thus, ligands that have more than two cysteines can be modified by replacing the remaining cysteines with serines or other residues. The resulting muteins may be tested for the requisite biological activity as described above. As exemplified herein, conjugates containing bFGF muteins in which Cys⁷⁸ and Cys⁹⁶ have been replaced with serine residues have been prepared.

In other preferred embodiments, muteins of growth factor ligands lack proliferative ability, or cell migration inducement and the like.

To produce fusion protein, DNA encoding the receptor-binding internalized ligand is modified so that as long as internalization is not required, the ligand does not include any cysteines available for reaction. In preferred embodiments, DNA encoding an FGF polypeptide is linked to DNA encoding a nucleic acid binding domain. The DNA encoding the FGF polypeptide or other receptor-binding internalized ligand is modified in order to remove the translation stop codon and other transcriptional or translational stop signals that may be present and to remove or replace DNA encoding the available cysteines. The DNA is then ligated to the DNA encoding the nucleic acid binding domain polypeptide directly or via a linker region between the first codon of the nucleic acid binding domain and the last codon of the FGF. The size of the linker region is not limited as long as the resulting conjugate binds and is internalized by a target cell. Presently, spacer regions of from about one to about seventy-five to ninety codons are preferred. The order of the receptor-binding internalized ligand and nucleic acid binding domain in the fusion protein may be reversed. If the nucleic acid binding domain is N-terminal, then it is modified to remove the stop codon and any stop signals.

As discussed above, any ligand for tumor cells may be modified and expressed in accord with the methods herein. The DNA encoding the resulting receptor-binding internalized ligand-nucleic acid binding domain can be inserted into a plasmid and expressed in a selected host, as described above, to produce a monogenous preparation. Fusion proteins of FGF-2 and protamine are exemplified herein.

Multiple copies of the modified receptor-binding internalized ligand/nucleic acid binding domain chimera can be inserted into a single plasmid in operative linkage with one promoter. When expressed, the resulting protein will be a multimer. Typically two to six copies of the chimera are inserted, preferably in a head to tail fashion, into one plasmid.

By way of example, DNA encoding human FGF2 has been mutagenized as described in the Examples using splicing by overlap extension (SOE). The DNA is modified by replacing the cysteines at positions 78 and 96 with serine. The codons encoding cysteine residues at positions 78 and 96 of FGF were converted to serine codons by SOE. Each application of the SOE method uses two amplified oligonucleotide products, which have complementary ends as primers and which include an altered codon at the locus at which the mutation is desired, to produce a hybrid product. A second amplification reaction that uses two primers that anneal at the non-overlapping ends amplify the hybrid to produce DNA that has the desired alteration.

2. Chemical Conjugation

a) Preparation of Receptor-binding Internalized Ligands

Receptor-binding internalized ligands are prepared as discussed above by any suitable method, including recombinant DNA technology, isolation from a suitable source, purchase from a commercial source, or chemical synthesis. The selected linker or linkers is (are) linked to the receptor-binding internalized ligands by chemical reaction, generally relying on an available thiol or amine group on the receptor-binding internalized ligands. Heterobifunctional linkers are particularly suited for chemical conjugation. Alternatively, if the linker is a peptide linker, then the linker may be incorporated into the ligand as a fusion protein. Although any of the ligands may be conjugated in this manner, FGF and VEGF conjugation are discussed merely by way of example and not by way of limitation.

If necessary or desired, the heterogeneity of preparations of ligand (e.g., FGF) containing chemical conjugates and fusion proteins can be reduced by modifying the ligand by deleting or replacing a site(s) that causes the heterogeneity. Such sites are typically cysteine residues that upon folding of the protein remain available for interaction with other cysteines or for interaction with more than one cytotoxic molecule per molecule of ligand. Thus, such cysteine residues do not include any cysteine residue that is required for proper folding or for binding to a receptor and subsequent internalization. For chemical conjugation, one cysteine residue that in physiological conditions is available for interaction is not replaced but is used as the site for linking the cytotoxic moiety. The resulting modified ligand is thus conjugated with a single species of nucleic acid binding domain (or nucleic acid).

If necessary, the contribution of each cysteine to the ability to bind to its receptors may be determined empirically. Each cysteine residue may be systematically replaced with a conservative amino acid change or deleted. The resulting mutein is tested for the requisite biological activity of the ability to bind to the receptor and internalize. If the mutein retains at least 50% of wild-type activity, then the cysteine residue is not required. Additional cysteines are systematically deleted and replaced and the resulting muteins are tested for activity. In this manner the minimum number and identity of the cysteines needed for receptor binding and internalization may be determined. Retention of proliferative activity is indicative, though not definitive, of the retention of activity of a ligand that is a growth factor. Proliferative activity may be measured by any suitable proliferation assay, such as the assay for FGF, exemplified below, that measures the increase in cell number of bovine aortic endothelial cells.

It is noted, however, that modified or mutant FGFs or other growth factos may exhibit reduced or no proliferative activity, but are suitable for use herein, if they retain the ability to target cytocide-encoding agent to cells bearing FGF receptors and result in internalization. Certain residues of FGF-2 have been associated with proliferative activity. Modification of these residues Arg 116, Lys 119, Tyr 120, Trp 123 to Ile 116, Glu 119, Ala 120, Ala 123 may be made individually (see SEQ ID NO. 25) to remove this function. In addition, a double modification of Arg 118 to Lys ard Lys 119 to Glu also gives markedly reduced proliferative ability. The resulting protein is tested for proliferative activity by a standard assay.

The cysteine residues in the FGF family that may be essential for retention of biological activity and that are not preferred residues for deletion or replacement are as follows: FGF-2, cys¹⁰¹; FGF-3, cys¹¹⁵; FGF-4, cys¹⁵⁵; FGF-5, cys¹⁶⁰; FGF-6, cys¹⁴⁷; FGF-7, cys¹³⁷; FGF-8, cys¹²⁷; and FGF-9, cys¹³⁴. For example, FGF-1 has cysteines at positions 31, 98 and 132; FGF-2 has cysteines at positions 34, 78, 96 and 101; FGF-3 has cysteines at positions 50 and 115; FGF-4 has cysteines at positions 88 and 155; FGF-5 has cysteines at positions 19, 93, 160 and 202; FGF-6 has cysteines at positions 80 and 147; FGF-7 has cysteines at positions 18, 23, 32, 46, 71, 133 and 137; FGF-8 has cysteines at positions 10, 19, 109 and 127; and FGF-9 has cysteines at positions 68 and 134.

Since FGF-3, FGF-4 and FGF-6 have only two cysteines, for purposes of chemical conjugation, preferably neither cysteine is deleted or replaced, unless another residue, preferably one near either terminus, is replaced with a cysteine. With respect to the other FGF family members, at least one cysteine must remain available for conjugation with the cytotoxic conjugate. A second cysteine may be required to form a disulfide bond. Thus, any FGF or other peptide that has more than three cysteines is modified for chemical conjugation by deleting or replacing the other cysteine residues. Peptides that have three cysteine residues are modified by elimination of one cysteine. conjugated to a cytotoxic moiety and tested for the ability to bind to receptor and internalize the cytotoxic moiety.

In accord with the methods herein, several muteins of basic FGF for chemical conjugation have been produced (preparation of muteins for recombinant expression of the conjugate is described below). DNA, obtained from pFC80 (see PCT Application Ser. No. PCT/US93/05702; U.S. application Ser. No. 07/901,718) encoding basic FGF has been mutagenized. Mutagenesis of cysteine 78 of basic FGF (FGF-2) to serine ([C78S]FGF) or cysteine 96 to serine ([C96S]FGF) produced two mutants that retain virtually complete proliferative activity of native basic FGF as judged by the ability to stimulate endothelial cell proliferation in culture. The activities of the two mutants and the native protein do not significantly differ as assessed by efficacy or maximal response. Sequence analysis of the modified DNA verified that each of the mutants has one codon for cysteine converted to that for serine. The construction and biological activity of FGF-1 with cysteine substitutions of one, two or all three cysteines has been disclosed (U.S. Pat. No. 5,223,483). The mitogenic activity of the mutants was similar to or increased over the native protein. Thus, any of the cysteines may be mutated and FGF-1 will still bind and be internalized.

The resulting mutein or unmodified peptide is reacted with a nucleic acid binding domain and optionally a linker. The muteins may react with a single species of derivatized nucleic acid binding domain (mono-derivatized nucleic acid binding domain), thereby resulting in monogenous and homogeneous preparations of ligand-nucleic acid binding domain conjugates. The resulting chemical conjugates do not aggregate and retain the requisite biological activities.

In the case of VEGF, VEGF₁₂₁ contains 9 cysteines and each of VEGF₁₆₅, VEGF₁₈₉ and VEGF₂₀₆ contain 7 additional residues in the region not present in VEGF₁₂₁. Any of the 7 are likely to be non-essential for targeting and internalization of linked cytotoxic agents. Recently, the role of Cys-25, Cys-56, Cys-67, Cys-101, and Cys-145 in dimerization and biological activity was assessed (Claffery et al., Biochem. Biophys. Acta 1246:1-9, 1995). Dimerization requires Cys-25, Cys-56, and Cys-67. Substitution of any one of these cysteine residues resulted in secretion of a monomeric VEGF, which was inactive in both vascular permeability and endothelial cell mitotic assays. In contrast, substitution of Cys 145 had no effect on dimerization, although biological activities were somewhat reduced. Substitution of Cys-101 did not result in the production of a secreted or cytoplasmic protein. Thus, substitution of Cys-145 is preferred.

The VEGF monomers are preferably linked via non-essential cysteine residues to the linkers or to the nucleic acid binding domain. VEGF that has been modified by introduction of a Cys residue at or near one terminus, preferably the N-terminus is preferred for use in chemical conjugation. For use herein, preferably the VEGF is dimerized prior to linkage to the linker and/or nucleic acid binding domain. Methods for coupling proteins to the linkers, such as the heterobifunctional agents, or to nucleic acids, or to proteins are known to those of skill in the art and are also described herein.

Methods for chemical conjugation of proteins are known to those of skill in the art. The preferred methods for chemical conjugation depend on the selected components, but preferably rely on disulfide bond formation. For example, if the targeted agent is SPDP-derivatized saporin, then it is advantageous to dimerize the VEGF moiety prior coupling or conjugating to the derivatized saporin. If VEGF is modified to include a cysteine residue at or near the N-, preferably, or C- terminus, then dimerization should follow coupling to the nucleic acid binding domain.

b) Preparation of NABD

A nucleic acid binding domain is prepared for chemical conjugation. For chemical conjugation, a nucleic acid binding domain may be derivatized with SPDP or another suitable chemical. If the binding domain does not have a Cys residue available for reaction, one can be either inserted or substituted for another amino acid. If desired, mono-derivatized species may be isolated, essentially as described.

For chemical conjugation, the nucleic acid binding domain may be derivatized or modified such that it includes a cysteine residue for conjugation to the receptor-binding internalized ligand. Typically, derivatization proceeds by reaction with SPDP, which results in a heterogeneous population. For example, a nucleic acid binding domain that is derivatized by SPDP to a level of 0.9 moles pyridine-disulfide per mole of nucleic acid binding domain includes a population of non-derivatized, mono-derivatized and di-derivatized proteins. Nucleic acid binding domain proteins, which are overly derivatized with SPDP, may lose ability to bind nucleic acid because of reaction with sensitive lysines (Lambert et al., Cancer Treat. Res. 37:175-209, 1988).

The use of purified mono-derivatized nucleic acid binding domain has distinct advantages over the non-purified material. The amount of receptor-binding internalized ligand that can react with nucleic acid binding domain is limited to one molecule with the mono-derivatized material. The quantity of non-derivatized nucleic acid binding domain in the preparation of the non-purified material can be difficult to judge and this may lead to errors in being able to estimate the correct proportion of derivatized nucleic acid binding domain to add to the reaction mixture. However, because of the removal of a negative charge by the reaction of SPDP with lysine, the various species, exhibit charge differences. This charge difference may be exploited in the purification of mono-derivatized nucleic acid binding domain by Mono-S cation exchange chromatography. There may still be sources of heterogeneity with the mono-derivatized nucleic acid binding domain used here but is acceptable as long as binding to the cytocide-encoding agent is not impacted. For example, because more than one amino group on the nucleic acid binding domain may react with the succinimidyl moiety, it is possible that more than one amino group on the surface of the protein is reactive.

As an alternative to derivatizing to introduce a sulfhydryl, the nucleic acid binding domain can be modified by the introduction of a cysteine residue by well known recombinant methods. Preferred loci for introduction of a cysteine residue include the N-terminus region, preferably within about one to twenty residues from the N-terminus of the nucleic acid binding domain.

Using either methodology (reacting mono-derivatized nucleic acid binding domain or introducing a Cys residue into nucleic acid binding domain), the resulting preparations of chemical conjugates are monogenous and should be free of aggregates.

3. Linkers

As used herein, a “linker” is a chemical or peptide that links a receptor-binding internalized ligand or fragment thereof and nucleic acid binding domain. In certain instances, a linker is used to conjugate the ligand directly to the nucleic acid. The linkers provided herein confer specificity, enhance intracellular availability, serum stability and/or solubility on the conjugate and may serve to promote condensation of nucleic acids for delivery to a cell.

The linkers provided herein confer specificity and serum stability on a conjugate, for example, by providing a substrate for certain proteases, particularly proteases that are present in only certain subcellular compartments or that are present at higher levels in tumor cells than normal cells. Specificity for proteases that are present in intracellular compartments and absent in blood is particularly preferred. The linkers may also include sorting signals such as cytoplasmic retention signals that direct the conjugate to particular intracellular loci or compartments. Additionally, the linkers may reduce steric hindrance between the ligand and other protein or linked nucleic acid by distancing the components of the conjugate. Linkers may also condense the nucleic acid. For this purpose, the linker comprises highly basic amino acids (e g, Lys, Arg) and may even be poly-L-lysine or poly-D-lysine.

In order to provide serum stability, increase solubility and/or intracellular concentration or condense nucleic acid, one or more linkers (are) inserted between the receptor-binding internalized ligand and the nucleic acid binding domain or nucleic acid. These linkers include peptide linkers, such as a substrate for an intracellular protease, and chemical linkers, such as acid labile linkers, ribozyme substrate linkers and others. Peptides linkers may be inserted using heterobifunctional reagents, described below, or, are linked to a ligand and nucleic acid binding domain by recombinant means. Chemical linkers are inserted by covalently coupling the linker to the ligand (e.g., FGF, other growth factor protein, or cytokine) and the nucleic acid binding domain, at the N- or C-terminus or an internal residue. Heterobifunctional agents, described below, may be used to effect such covalent coupling.

a) Protease substrates

Peptides encoding protease-specific substrates may be introduced between the ligand and the nucleic acid binding domain. The peptides may be inserted using heterobifunctional reagents, for example, or inserted by recombinant means and expression of the resulting chimera.

Any protease specific substrate (see, e.g., O'Hare et al., FEBS 273:200-204, 1990; Forsberg et al., J. Protein Chem. 10:517-526, 1991; Westby et al., Bioconjugate Chem. 3:375-381, 1992) may be used as a linker as long as the substrate is cleaved in an intracellular compartment. Preferred substrates include those that are specific for proteases that are expressed at higher levels in tumor cells, that are preferentially expressed in the endosome, or that are absent in blood. The following substrates are suitable for use in the present invention: cathepsin B substrate, cathepsin D substrate, trypsin substrate, thrombin substrate, and recombinant subtilisin substrate.

b) Flexible Linkers

Flexible linkers, which reduce steric hindrance and increase in vitro solubility of the conjugates are contemplated for use, either alone or with other linkers, such as the protease specific substrate linkers. Typically, these linkers are simple polymers of small amino acids (i e., small side groups) with uncharged polar side groups. These amino acids (Gly, Ser, Thr, Cys, Tyr, Asn, Gln) are more soluble in water. Of these amino acids, Gly and Ser are preferred. Such linkers include, but are not limited to, (Gly₄Ser)ₙ, (Ser₄Gly)ₙ and (AlalaProAla)ₙ in which n is 1 to 6, preferably 1-4, such as:

Gly₄Ser SEQ ID NO.26
CCATGGGCGG CGGCGGCTCT GCCATGG
(Gly₄Ser)₂ SEQ ID NO.27
CCATGGGCGG CGGCGGCTCT GGCGGCGGCG GCTCTGCCAT GG
(Ser₄Gly)₄ SEQ ID NO.28
CCATGGCCTC GTCGTCGTCG GGCTCGTCGT CGTCGGGCTC GTCGTCGTCG GGCTCGTCGT CGTCGGGCGC CATGG
(Ser₄Gly)₂ SEQ ID NO.29
CCATGGCCTC GTCGTCGTCG GGCTCGTCGT CGTCGGGCGC CATGG
(AlaAlaProAla)ₙ, where n is 1 to 4, preferably 2 (see SEQ ID NO.30)

c) Heterobifunctional Cross-linking Reagents

Numerous heterobifunctional cross-linking reagents may be used to form covalent bonds between amino groups and thiol groups and to introduce thiol groups into proteins, (see, e.g., the PIERCE CATALOG, ImmunoTechnology Catalog & Handbook, 1992-1993, which describes the preparation of and use of such reagents and provides a commercial source for such reagents; see also, e.g., Cumber et al., Bioconjugate Chem. 3:397-401, 1992; Thorpe et al., Cancer Res. 47:5924-5931, 1987; Gordon et al., Proc. Natl. Acad Sci. 84:308-312, 1987; Walden et al., J. Mol. Cell Immunol. 2:191-197, 1986; Carlsson et al., Biochem. J. 173:723-737, 1978; Mahan et al., Anal. Biochem., 162:163-170, 1987; Wawryznaczak et al., Br. J. Cancer 66:361-366, 1992; Fattom et al., Infection & Immun. 60:584-589, 1992). These reagents may be used to form covalent bonds between the receptor-binding internalized ligands with protease substrate peptide linkers and nucleic acid binding domain. These reagents include, but are not limited to: N-succinimidyl-3-(2-pyridyldithio)propionate (SPDP; disulfide linker); sulfosuccinimidyl 6-[3-(2-pyridyldithio)propionamido]hexanoate (sulfo-LC-SPDP); succinimidyloxycarbonyl-α-methyl benzyl thiosulfate (SMBT, hindered disulfate linker); succinimidyl 6-[3-(2-pyridyldithio) propionamido]hexanoate (LC-SPDP); sulfosuccinimidyl 4-(N-maleimidomethyl)cyclohexane-1-carboxylate (sulfo-SMCC); succinimidyl 3-(2-pyridyldithio)butyrate (SPDB; hindered disulfide bond linker); sulfosuccinimidyl 2-(7-azido-4-methylcoumarin-3-acetamide) ethyl-1,3′-dithiopropionate (SAED); sulfosuccinimidyl 7-azido-4-methylcoumarin-3-acetate (SAMCA); sulfosuccinimidyl 6-[alpha-methyl-alpha-(2-pyridyldithio)toluamido]hexanoate (sulfo-LC-SMPT); 1,4-di-[3′-(2′-pyridyldithio)propionamido]butane (DPDPB); 4-succinimidyloxycarbonyl- -methyl- -(2-pyridylthio)toluene (SMPT, hindered disulfate linker); sulfosuccinimidyl6[-methyl- -2-pyridyldithio)toluamido]hexanoate (sulfo-LC-SMPT); m-maleimidobenzoyl-N-hydroxysuccinimide ester (MBS); m-maleimidobenzoyl-N-hydroxysulfosuccinimide ester (sulfo-MBS); N-succinmidyl(4-iodoacetyl)aminobenzoate (SIAB; thioether linker); sulfosuccinimidyl(4-iodoacetyl)amino benzoate (sulfo-SIAB); succinimidyl4(p-maleimidophenyl)butyrate (SMPB); sulfosuccinimidyl4-(p-maleimidophenyl)butyate (sulfo-SMPB); azidobenzoyl hydrazide (ABH).

These linkers should be particularly useful when used in combination with peptide linkers, such as those that increase flexibility.

d) Acid Cleavable, Photocleavable, and Heat Sensitive Linkers

Acid cleavable linkers include, but are not limited to, bismaleimideothoxy propane, adipic acid dihydrazide linkers (see, e.g., Fattom et al., Infection & Immune 60:584-589, 1992) and acid labile transferrin conjugates that contain a sufficient portion of transferrin to permit entry into the intracellular transferrin cycling pathway (see, e.g., Welhöner et al., J. Biol. Chem. 266:4309-4314, 1991). Conjugates linked via acid cleavable linkers should be preferentially cleaved in acidic intracellular compartments, such as the endosome.

Photocleavable linkers are cleaved upon exposure to light (see, e.g., Goldmacher et al., Bioconj. Chem. 3:104-107, 1992), thereby releasing the targeted agent upon exposure to light. (Hazum et al., Proc. Eur. Pept. Symp., 16th, Brunfeldt, K (Ed), pp. 105-110, 1981; nitrobenzyl group as a photocleavable protective group for cysteine; Yen et al., Makromol. Chem 190:69-82, 1989; water soluble photocleavable copolymers, including hydroxypropylmethacrylamide copolymer, glycine copolymer, fluorescein copolymer and methykhodamine copolymer; and Senter et al., Photochem. Photobiol. 42:231-237, 1985; nitrobenzyloxycarbonyl chloride cross linking reagents that produce photocleavable linkages). Such linkers are particularly useful in treating dermatological or ophthalmic conditions. In addition, other tissues, such as blood vessels that can be exposed to light using fiber optics during angioplasty in the prevention or treatment of restenosis may benefit from the use of photocleavable linkers. After administration of the conjugate, the eye or skin or other body part is exposed to light, resulting in release of the targeted moiety from the conjugate. Heat sensitive linkers would also have similar applicability.

B. Preparation of Ligand-nucleic Acid Conjugates

As an alternative, the receptor-internalized binding ligand may be conjugated to the nucleic acid, either directly or through a linker. Methods for conjugating nucleic acids, at the 5′ ends, 3′ ends and elsewhere, to the amino and carboxyl termini and other sites in proteins are known to those of skill in the art (for a review see, e.g., Goodchild, (1993) In: Perspectives in Bioconjugate Chemistry, Mears, Ed., American Chemical Society, Washington, D.C. pp. 77-99). For example, proteins have been linked to nucleic acids using ultraviolet irradiation (Sperling et al. (1978) Nucleic Acids Res. 5:2755-2773; Fiser et al. (1975) FEBS Lett. 52:281-283), bifunctional chemicals (Bäumert et al. (1978) Eur. J. Biochem. 89:353-359; and Oste et al. (1979) Mol. Gen. Genet. 168:81-86) and photochemical cross-linking (Vanin et al. (1981) FEBS Lett. 124:89-92; Rinke et al. (1980) J. Mol. Biol. 137:301-314; Millon et al. (1980) Eur. J. Biochem. 110:485-454).

In particular, the reagents (N-acetyl-N′-(p-glyoxylylbenzolyl)cystarnine and 2-iminothiolane have been used to couple DNA to proteins, such as α-macroglobulin (α₂M) via mixed disulfide formation (see Cheng et al., Nucleic Acids Res. 11:659-669, 1983). N-acetyl-N′-(p-glyoxylylbenzolyl)cystamine reacts specifically with nonpaired guaninine residues and, upon reduction, generates a free sulfhydryl group. 2-iminothiolane reacts with proteins to generate sulfhydryl groups that are then conjugated to the derivatized DNA by an intermolecular disulfide interchange reaction. Any linkage may be used provided that the targeted nucleic acid is active upon internalization of the conjugate. Thus, it is expected that cleavage of the linkage may be necessary, although it is contemplated that for some reagents, such as DNA encoding ribozymes linked to promoters or DNA encoding therapeutic agents for delivery to the nucleus, such cleavage may not be necessary.

Thiol linkages, which are preferred, can be readily formed using heterbiofunctional reagents. Such linkages are reversible in a cell to release the nucleic acid from the ligand. Amines have also been attached to the terminal 5′ phosphate of unprotected oligonucleotides or nucleic acids in aqueous solutions by reacting the nucleic acid with a water-soluble carbodiimide, such as 1-ethyl-3′[3-dimethylaminopropyl]carbodiimide (EDC) or N-ethyl-N′(3-dimethylaminopropylcarbodiimidehydrochloride (EDCI), in imidazole buffer at pH 6 to produce the 5′phosphorimidazolide. Contacting the 5′phosphorimidazolide with amine-containing molecules, such as an FGF, and ethylenediamine, results in stable phosphoramidates (see, e.g., Chu et al., Nucleic Acids Res. 11:6513-6529, 1983; and WO 88/05077). In particular, a solution of DNA is saturated with EDC, at pH 6 and incubated with agitation at 4° C. overnight. The resulting solution is then buffered to pH 8.5 by adding, for example about 3 volumes of 100 mM citrate buffer, and adding about 5 μg—about 20 μg of an FGF, and agitating the resulting mixture at 4° C. for about 48 hours. The unreacted protein may be removed from the mixture by column chromatography using, for example, Sephadex G75 (Pharmacia) using 0.1 M ammonium carbonate solution, pH 7.0 as an eluting buffer. The isolated conjugate may be lyophilized and stored until used.

U.S. Pat. No. 5,237,016 provides methods for preparing nucleotides that are bromacetylated at their 5′ termini and reacting the resulting oligonucleotides with thiol groups. Oligonucleotides derivatized at their 5′-termini bromoacetyl groups can be prepared by reacting 5′-aminohexyl-phosphoramidate oligonucleotides with bromoacetic acid-N-hydroxysuccinimide ester as described in U.S. Pat. No. 5,237,016. This patent also describes methods for preparing thiol-derivatized nucleotides, which can then be reacted with thiol groups on the selected growth factor. Briefly, thiol-derivatized nucleotides are prepared using a 5′-phosphorylated nucleotide in two steps: (1) reaction of the phosphate group with imidazole in the presence of a diimide and displacement of the imidazole leaving group with cystamine in one reaction step; and reduction of the disulfide bond of the cystamine linker with dithiothreitol (see, also, Orgel et al. ((1986) Nucl Acids Res. 14:6511, which describes a similar procedure). The 5′-phosphorylated starting oligonucleotides can be prepared by methods known to those of skill in the art (see, eg, Maniatis et al. (1982) Molecular Cloning: A Laboratory Manual, Cold Spring Harbor Laboratory, New York, p. 122).

The nucleic acid, such as a methylphosphonate oligonucleotide (MP-oligomer), may be derivatized by reaction with SPDP or SMPB. The resulting MP-oligomer may be purified by HPLC and then coupled to an FGF, such as an FGF or FGF mutein, modified by replacement of one or more cysteine residues, as described above. The MP-oligomer (about 0.1 μM) is dissolved in about 40-50 μl of 1:1 acetonitrile/water to which phosphate buffer (pH 7.5, final concentration 0.1 M) and a 1 mg MP-oligomer in about 1 ml phosphate buffered saline is added. The reaction is allowed to proceed for about 5-10 hours at room temperature and is then quenched with about 15 μL 0.1 iodoacetamide. FGF-oligonucleotide conjugates can be purified on heparin sepharose Hi Trap columns (1 ml, Pharmacia) and eluted with a linear or step gradient. The conjugate should elute in 0.6 M NaCl.

The ligand may be conjugated to the nucleic acid construct encoding the cytocide or cytotoxic agent or may be conjugated to a mixture of oligonucleotides complementary to one strand of the construct. The oligonucleotides are then added to single stranded construct produced by melting a double-stranded construct or grown and isolated as single-stranded. As a general guideline, the oligonucleotides should hybridize at a higher temperature than the construct alone, if a double-stranded construct is used as the starting material. The gaps are filled in by DNA polymerase I to generate a construct with one strand conjugated to ligand and one strand unconjugated. Oligonucleotides conjugated to ligand and complementary to the other strand may be used in addition to generate a mixture of constructs with different strands linked to ligand. Any remaining single stranded plasmid may be digested with a single strand specific endonuclease. The ligand-conjugated constructs are then mixed with a nucleic acid binding domain, such as protamine or polylysine, to effect condensation of the construct for delivery. Optimal ratios of ligand to DNA may be determined experimentally by receptor-mediated transfection of a construct containing a reporter gene.

As discussed above, for efficient uptake by a cell, the nucleic acid is in a toroidal shape. If the linker portion of the conjugate does not cause the proper condensation, then a molecule, such as poly-L-lysine, poly-D-lysine, spermine or the like, is added. Testing for toroids and receptor binding and internalization is described above.

C. Complex and Toroid Formation

The receptor-binding internalized ligand/nucleic acid binding domain is incubated with the cytocide-encoding or prodrug-encoded agent, preferably a circular DNA molecule, to be delivered under conditions that allow binding of the nucleic acid binding domain to the agent. Conditions will vary somewhat depending on the nature of the nucleic acid binding domain, but will typically occur in 0.1 M NaCl and 20 mM HEPES or other similar buffer. Alternatively, salt conditions can be varied to increase the packing or condensation of DNA. The extent of binding is preferably tested for each preparation. After complexing, additional nucleic acid binding domain, such as poly-L-lysine, may be added to further condense the nucleic acid.

In addition to binding of the nucleic acid binding domain to nucleic acid, the complex needs to be condensed for efficient uptake by a cell. A toroidal shape allows efficient uptake, whereas rod shapes are not efficiently taken up. Thus, conditions for binding that favor the formation of toroids are preferred. If a nucleic acid binding domain itself does not cause toroid formation, an addition composition that causes toroid formation should be added. Such compositions include poly-L-lysine, poly-D-lysine, spermine, spermidine, cobalt hexamine, MnCl₂, protamines and the like.

For preparing complexes, the nucleic acid is diluted and added to the conjugate with gentle agitation, so as not to cause frothing. The length of DNA is irrelevant to toroid formation.

By way of example, test constructs have been made and tested. One construct is a chemical conjugate of FGF2 and poly-L-lysine. The FGF2 molecule is a variant in which the Cys residue at position 96 has been changed to a serine; thus, only the Cys at position 78 is available for conjugation. This FGF2 is called FGF2-3. The poly-L-lysine was derivatized with SPDP and coupled to FGF2-3. This FGF2-3/poly-L-lysine conjugate was used to deliver a plasmid able to express the β-galactosidase gene.

The ability of a construct to bind nucleic acid molecules may be conveniently assessed by agarose gel electrophoresis. Briefly, a plasmid, such as pSVβ, is digested with restriction enzymes to yield a variety of fragment sizes. For ease of detection, the fragments may be labeled with 32p either by filling in of the ends with DNA polymerase I or by phosphorylation of the 5′-end with polynucleotide kinase following dephosphorylation by alkaline phosphatase. The plasmid fragments are then incubated with the receptor-binding internalized ligand/nucleic acid binding domain in this case, FGF2-3/poly-L-lysine in a buffered saline solution, such as 20 mM HEPES, pH 7.3, 0.1M NaCl. The reaction mixture is electrophoresed on an agarose gel alongside similarly digested, but nonreacted fragments. If a radioactive label was incorporated, the gel may be dried and autoradiographed. If no radioactive label is present, the gel may be stained with ethidium bromide and the DNA visualized through appropriate red filters after excitation with UV. Binding has occurred if the mobility of the fragments is retarded compared to the control. In the example case, the mobility of the fragments was retarded after binding with the FGF2-3/poly-L-lysine conjugate. If there is insufficient binding, poly-L-lysine may be additionally added until binding is observed.

The amount of compaction and shape of compaction may be measured in several different ways. Visualization by electron microscopy, measurement of circular dichroism, and laser light scatterings can all distinguish toroids from rods.

Further testing of the conjugate is performed to show that it binds to the cell surface receptor and is internalized into the cell. It is not necessary that the receptor-binding internalized ligand part of the conjugate retain complete biological activity. For example, FGF is mitogenic on certain cell types. As discussed above, this activity may not always be desirable. If this activity is present, a proliferation assay is performed. Likewise, for each desirable activity, an appropriate assay may be performed. However, for application of the subject invention, the only criteria that need be met are receptor binding and internalization.

Receptor binding and internalization may be measured by the following three assays. (1) A competitive inhibition assay of the complex to cells expressing the appropriate receptor demonstrates receptor binding. (2) Receptor binding and internalization may be assayed by measuring expression of a reporter gene, such as β-gal (e.g., enzymatic activity), in cells that have been transformed with a complex of a plasmid encoding a reporter gene and a conjugate of a receptor-binding internalized ligand and nucleic acid binding domain. This assay is particularly useful for optimizing conditions to give maximal transformation. Thus, the optimum ratio of receptor-binding internalized ligand/nucleic acid binding domain to nucleic acid and the amount of DNA per cell may readily be determined by assaying and comparing the enzymatic activity of β-gal. As such, these first two assays are useful for preliminary analysis and failure to show receptor binding or β-gal activity does not per se eliminate a candidate receptor-binding internalized ligand/nucleic acid binding domain conjugate or fusion protein from further analysis. (3) The preferred assay is a cytotoxicity assay performed on cells transformed with a cytocide-encoding agent bound by receptor-binding internalized ligand/nucleic acid binding domain. While, in general, any cytocidal molecule may be used, ribosome inactivating proteins are preferred and saporin, or another type I ribosome inactivating protein, is particularly preferred. A statistically significant reduction in cell number demonstrates the ability of the receptor-binding internalized ligand/nucleic acid binding domain conjugate or fusion to deliver nucleic acids into a cell. Any cell expressing the appropriate receptor may be used. For FGF as a ligand, cell lines including COS and rabbit smooth muscle cells may be used.

1. Endosome-disruptive Peptides

In addition, or alternatively, membrane-disruptive peptides may be incorporated into the complexes. For example, adenoviruses are known to enhance disruption of endosomes. Virus-free viral proteins, such as influenza virus hemagglutinin HA-2, also disrupt endosomes and are useful in the present invention. Other proteins may be tested in the assays described herein to find specific endosome disrupting agents that enhance gene delivery. In general, these proteins and peptides are amphipathic (see Wagner et al., Adv. Drug. Del. Rev. 14:113-135, 1994).

Endosome-disruptive peptides, sometimes called fusogenic peptides, may be incorporated into the complex of receptor-internalized binding ligand, nucleic acid binding domain, and cytocide-encoding agent. Two such peptides derived from influenza virus are: GLFEAIEGFIENGWEGMIDGGGC (SEQ ID NO. 31) and GLFEAIEGFIENGWEGMIDGWYGC (SEQ ID NO. 32). Other peptides useful for disrupting endosomes may be identified by general characteristics: 25-30 residues in length, contain an alternating pattern of hydrophobic domains and acidic domains, and at low pH (e.g., pH 5) form amphipathic a-helices. A candidate endosome-disrupting peptide is tested by incorporating it into the complex and determining whether it increases the total number of cells expressing the target gene. The peptides are added to a complex having excess negative charge. For example, a DNA construct is complexed with an FGF-poly-L-lysine chemical conjugate so that only a portion of the negative charge of the DNA is neutralized. Poly-L-lysine is added to further bind the DNA and a fusogenic peptide is then added. Optimal ratios of DNA, poly-L-lysine and fusogenic peptide are determined using assays, such as gene expression and cell viability.

The fusogenic peptides may alternatively be incorporated into the complex as a fusion protein with either the ligand or the nucleic acid binding domain or both. The endosome-disruptive peptide may be present as single or multiple copies at the N- or C-terminus of the ligand. A single fusion protein of the endosome-disruptive peptide, nucleic acid binding domain, and receptor-internalized binding ligand may be constructed and expressed. For insertion into a construct, DNA encoding the endosome-disruptive peptide may be synthesized by PCR using overlapping oligonucleotides and incorporating a restriction site at the 5′ and 3′ end to facilitate cloning. The sequence may be verified by sequence analysis.

2. Adenovirus or Viral Proteins

To enhance endosomolysis, adenovirus or adenoviral proteins can be incorporated into the complexes (U.S. Pat. No. 5,547,932). The virus or viral proteins are internalized into the cells together with the complex and promote release of the contents of endosomes into the cytoplasm. Other viruses, such as rhinoviruses and influenza virus may also be used (Ibid.). Preferably, when whole virus is used, the virus is inactivated.

VII. Formulation and Administration of Pharmaceutical Compositions

The conjugates and complexes provided herein are useful in the treatment and prevention of various diseases tumors. As used herein, “treatment” means any manner in which the symptoms of a condition, disorder or disease are ameliorated or otherwise beneficially altered. Treatment also encompasses any pharmaceutical use of the compositions herein. As used herein, “amelioration” of the symptoms of a particular disorder refers to any lessening, whether permanent or temporary, lasting or transient, that can be attributed to or associated with administration of the composition.

A. Treatment of Tumors

As noted above, the compositions of the present invention are used to treat tumors. In these diseases, cell growth is excessive or uncontrolled. Tumors suitable for treatment within the context of this invention include, but are not limited to, breast tumors, gliomas, melanomas, prostate cancer, hepatomas, sarcomas, lymphomas, leukemias, ovarian tumors, thymomas, nephromas, pancreatic cancer, colon cancer, head and neck cancer, stomach cancer, lung cancer, mesotheliomas, myeloma, neuroblastoma, retinoblastoma, cervical cancer, uterine cancer, and squamous cell carcinoma of skin. As discussed above, ligands for these cancers bind to cell surface receptors that are generally preferentially expressed in tumors. Many of these cell surface receptors and their ligands are known. For tumors without such ligand-receptor pairs, ligands, such as antibodies, can be developed.

Through delivery of the compositions of the present invention, unwanted growth of cells may be slowed or halted, thus ameliorating the disease. The methods utilized herein specifically target and kill or halt proliferation of tumor cells having receptors for the ligand on their surfaces. This treatment is suitable for warm-blooded animals: mammals, including, but not limited to, humans, horses, dogs, and cats, and for non-mammals, such as avian species. Methods of treating such animals with these FGF conjugates are provided herein. These conjugates are shown to be effective against tumors, as well as against other pathophysiological conditions caused by a proliferation of cells which are sensitive to FF mitogenic stimulation.

B. Preparation of Pharmaceutical Agents

Pharmaceutical carriers or vehicles suitable for administration of the conjugates and complexes provided herein include any such carriers known to those skilled in the art to be suitable for the particular mode of administration. In addition, the conjugates and complexes may be formulated as the sole pharmaceutically active ingredient in the composition or may be combined with other active ingredients.

The conjugates and complexes can be administered by any appropriate route, for example, orally, parenterally, including intravenously, intradermally, subcutaneously, or topically, in liquid, semi-liquid or solid form and are formulated in a manner suitable for each route of administration. Preferred modes of administration depend upon the indication treated. Dermatological and ophthalmologic indications will typically be treated locally; whereas, tumors and restenosis, will typically be treated by systemic, intradermal, or intramuscular modes of administration.

The conjugates and complexes herein may be formulated into pharmaceutical compositions suitable for topical, local, intravenous and systemic application. For ophthalmic uses, local administration, either by topical administration or by injection is preferred. Time release formulations are also desirable. Effective concentrations of one or more of the conjugates and complexes are mixed with a suitable pharmaceutical carrier or vehicle. As used herein an “effective amount” of a compound for treating a particular disease is an amount that is sufficient to ameliorate, or in some manner reduce the symptoms associated with the disease. Such amount may be administered as a single dosage or may be administered according to a regimen, whereby it is effective. The amount may cure the disease but, typically, is administered in order to ameliorate the symptoms of the disease. Repeated administration may be required to achieve the desired amelioration of symptoms.

As used herein, “an ophthalmically effective amount” is that amount which, in the composition administered and by the technique administered, provides an amount of therapeutic agent to the involved eye tissues sufficient to prevent or reduce corneal haze following excimer laser surgery, prevent closure of a trabeculectomy, prevent or substantially slow the recurrence of pterygii, and other conditions.

The concentrations or amounts of the conjugates and complexes that are effective requires delivery of an amount, upon administration, that ameliorates the symptoms or treats the disease. Typically, the compositions are formulated for single dosage administration. Therapeutically effective concentrations and amounts may be determined empirically by testing the conjugates and complexes in known in vitro and in vivo systems, such as those described here; dosages for humans or other animals may then be extrapolated therefrom.

The conjugate is included in the pharmaceutically acceptable carrier in an amount sufficient to exert a therapeutically useful effect in the absence of undesirable side effects on the patient treated. The conjugates may be delivered as pharmaceutically acceptable salts, esters or other derivatives of the conjugates include any salts, esters or derivatives that may be readily prepared by those of skill in this art using known methods for such derivatization and that produce compounds that may be administered to animals or humans without substantial toxic effects. It is understood that number and degree of side effects depends upon the condition for which the conjugates and complexes are administered. For example, certain toxic and undesirable side effects are tolerated when treating life-threatening illnesses, such as tumors, that would not be tolerated when treating disorders of lesser consequence. The concentration of conjugate in the composition will depend on absorption, inactivation and excretion rates thereof, the dosage schedule, and amount administered as well as other factors known to those of skill in the art.

Preferably, the conjugate and complex are substantially pure. As used herein, “substantially pure” means sufficiently homogeneous to appear free of readily detectable impurities as determined by standard methods of analysis, such as thin layer chromatography (TLC), gel electrophoresis, high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound.

The conjugates and complexes may be formulated for local or topical application, such as for topical application to the skin and mucous membranes, such as in the eye, in the form of gels, creams, and lotions and for application to the eye or for intracistemal or intraspinal application. Such solutions, particularly those intended for ophthalmic use, may be formulated as 0.01% -10% isotonic solutions, pH about 5-7, with appropriate salts. The ophthalmic compositions may also include additional components, such as hyaluronic acid. The conjugates and complexes may be formulated as aerosols for topical application (see, e.g., U.S. Pat. Nos. 4,044,126, 4,414,209, and 4,364,923).

Solutions or suspensions used for parenteral, intradermal, subcutaneous, or topical application can include any of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of toxicity such as sodium chloride or dextrose. Parental preparations can be enclosed in ampules, disposable syringes or multiple dose vials made of glass, plastic or other suitable material.

If administered intravenously, suitable carriers include physiological saline or phosphate buffered saline (PBS), and solutions containing thickening and solubilizing agents, such as glucose, polyethylene glycol, and polypropylene glycol and mixtures thereof. Liposomal suspensions may also be suitable as pharmaceutically acceptable carriers. These may be prepared according to methods known to those skilled in the art.

Upon mixing or addition of the conjugate(s) with the vehicle, the resulting mixture may be a solution, suspension, emulsion or the like. The form of the resulting mixture depends upon a number of factors, including the intended mode of administration and the solubility of the conjugate in the selected carrier or vehicle. The effective concentration is sufficient for ameliorating the symptoms of the disease, disorder or condition treated and may be empirically determined based upon in vitro and/or in vivo data, such as the data from the mouse xenograft model for tumors or rabbit ophthalmic model. If necessary, pharmaceutically acceptable salts or other derivatives of the conjugates and complexes may be prepared.

The active materials can also be mixed with other active materials, that do not impair the desired action, or with materials that supplement the desired action, including viscoelastic materials, such as hyaluronic acid, which is sold under the trademark HEALON (solution of a high molecular weight (MW of about 3 millions) fraction of sodium hyaluronate; manufactured by Pharmacia, Inc. see, e.g. U.S. Pat. Nos. 5,292,362, 5,282,851, 5,273,056, 5,229,127, 4,517,295 and 4,328,803), VISCOAT (fluorine-containing (meth)acrylates, such as, 1H,1H,2H,2H-hepta-decafluorodecylmethacrylate; see, e.g., U.S. Pat. Nos. 5,278,126, 5,273,751 and 5,214,080; commercially available from Alcon Surgical, Inc.), ORCOLON (see, e.g., U.S. Pat. No. 5,273,056; commercially available from Optical Radiation Corporation), methylcellulose, methyl hyaluronate, polyacrylamide and polymethacrylamide (see, e.g., U.S. Pat. No. 5,273,751). The viscoelastic materials are present generally in amounts ranging from about 0.5 to 5.0%, preferably 1 to 3% by weight of the conjugate material and serve to coat and protect the treated tissues. The compositions may also include a dye, such as methylene blue or other inert dye, so that the composition can be seen when injected into the eye or contacted with the surgical site during surgery.

The conjugates and complexes may be formulated for local or topical application, such as for topical application to the skin and mucous membranes, such as in the eye, in the form of gels, creams, and lotions and for application to the eye. Such solutions, particularly those intended for ophthalmic use, may be formulated as 0.01%-10% isotonic solutions, pH about 5-7, with appropriate salts. Suitable ophthalmic solutions are known (see, e.g., U.S. Pat. No. 5,116,868, which describes typical compositions of ophthalmic irrigation solutions and solutions for topical application). Such solutions, which have a pH adjusted to about 7.4, contain, for example, 90-100 mM sodium chloride, 4-6 mM dibasic potassium phosphate, 4-6 mM dibasic sodium phosphate, 8-12 mM sodium citrate, 0.5-1.5 mM magnesium chloride, 1.5-2.5 mM calcium chloride, 15-25 mM sodium acetate, 10-20 mM D,L,-sodium β-hydroxybutyrate and 5-5.5 mM glucose.

The conjugates and complexes may be prepared with carriers that protect them against rapid elimination from the body, such as time release formulations or coatings. Such carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others. For example, the composition may be applied during surgery using a sponge, such as a commercially available surgical sponges (see, e.g., U.S. Pat. Nos. 3,956,044 and 4,045,238; available from Weck, Alcon, and Mentor), that has been soaked in the composition and that releases the composition upon contact with the eye. These are particularly useful for application to the eye for ophthalmic indications following or during surgery in which only a single administration is possible. The compositions may also be applied in pellets (such as Elvax pellets(ethylene-vinyl acetate copolymer resin); about 1-5 μg of conjugate per 1 mg resin) that can be implanted in the eye during surgery.

If oral administration is desired, the conjugate should be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules or troches. Pharmaceutically compatible binding agents and adjuvant materials can be included as part of the composition.

The tablets, pills, capsules, troches and the like can contain any of the following ingredients, or compounds of a similar nature: a binder, such as microcrystalline cellulose, gum tragacanth and gelatin; an excipient such as starch and lactose, a disintegrating agent such as, but not limited to, alginic acid and corn starch; a lubricant such as, but not limited to, magnesium stearate; a glidant, such as, but not limited to, colloidal silicon dioxide; a sweetening agent such as sucrose or saccharin; and a flavoring agent such as peppermint, methyl salicylate, and fruit flavoring.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The conjugates and complexes can also be administered as a component of an elixir, suspension, syrup, wafer, chewing gum or the like. A syrup may contain, in addition to the active compounds, sucrose as a sweetening agent and certain preservatives, dyes and colorings and flavors.

The active materials can also be mixed with other active materials that do not impair the desired action, or with materials that supplement the desired action, such as cis-platin for treatment of tumors.

Finally, the compounds may be packaged as articles of manufacture containing packaging material, one or more conjugates and complexes or compositions as provided herein within the packaging material, and a label that indicates the indication for which the conjugate is provided.

C Administration

Typically a therapeutically effective dosage should produce a serum concentration of active ingredient of from about 0.1 ng/ml to about 500 μg/ml. The pharmaceutical compositions typically should provide a dosage of from about 0.01 mg/kg to about 100-2000 mg/kg of conjugate, depending upon the conjugate. Local application for ophthalmic disorders and dermatological disorders should provide about 1 ng up to 100 μg, preferably about 1 ng to about 10 μg, per single dosage administration. It is understood that the amount to administer will be a function of the conjugate selected, the indication treated, and possibly the side effects that will be tolerated.

Therapeutically effective concentrations and amounts may be determined for each application herein empirically by testing the conjugates and complexes in known in vitro and in vivo systems (e.g., murine, rat, rabbit, or baboon models), such as those described herein; dosages for humans or other animals may then be extrapolated therefrom. The rabbit eye model is a recognized model for studying the effects of topically and locally applied drugs (see, e.g., U.S. Pat. Nos. 5,288,735, 5,263,992, 5,262,178, 5,256,408, 5,252,319, 5,238,925, 5,165,952; see also Mirate et al., Curr. Eye Res. 1:491-493, 1981).

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

D. Testing of Constructs

The nucleic acid delivery vehicles ma) be assessed in any number of in vitro model systems. In particular, target cells are grown in culture and incubated with the nucleic acid delivery vehicle. The nucleic acid can encode a reporter, in which case, the reporter product is assayed, or encode a cytocidal product, in which case cell killing is measured. Moreover, any assayable gene product can be used. For reporter genes, a wide variety of suitable genes are available. Such reporters include β-galactosidase, alkaline phosphatse, β-glucuronidase, large T antigen, any protein for which an antibody exists or can be developed. The choice of a reporter depends, in part, upon the cells being tested. Alternatively, the nucleic acid can encode a cytocidal product. Such products include all those described herein. Saporin is preferred.

The delivery vehicles may be assessed in in vivo model systems. Generally, a xenogeneic tumor model system will be used, but other tumor model systems are useful as well. In the xenogenic system, an immunodeficient mouse, or other immunodeficient animal, is injected with tumor cells, such as human tumor cells. The nucelic acid delivery vehicle is administered and tumor growth is monitored. Any reduction of tumor growth is useful within the context of this invention.

The following examples are included for illustrative purposes only and are not intended to limit the scope of the invention.

### EXAMPLES

### Example 1

### Isolation of DNA Encoding Saporin

A. Materials and Methods

1. Bacterial Strains

E. coli strain JA221 (lpp⁻ hdsM+ trpE5 leuB6 lacY recAl F′[IacI^{q} lac⁺ pro⁺]) is available from the American Type Culture Collection (ATCC), Rockville, Md. 20852, under the accession number ATCC 33875 or from the Northern Regional Research Center (NRRL), Agricultural Research Service, U.S. Department of Agriculture, Peoria, Ill. 61604, under the accession number NRRL B-15211 (see also U.S. Pat. No. 4,757,013 to Inouye; and Nakamura el al., Cell 18:1109-1117, 1979). Strain INV1α is commercially available from Invitrogen, San Diego, Calif.

2. DNA Manipulations

The restriction and modification enzymes employed herein are commercially available in the U.S. Native saporin and rabbit polyclonal antiserum to saporin were obtained as previously described in Lappi et al., Biochem. Biophys. Res. Comm. 129:934-942. Ricin A chain is commercially available from Sigma, Milwaukee, Wis. Antiserun was linked to Affi-gel 10 (Bio-Rad, Emeryville, Calif.) according to the manufacturer's instructions. Sequencing was performed using the Sequenase kit of United States Biochemical Corporation (version 2.0) according to the manufacturer's instructions. Minipreparation and maxipreparation of plasmids, preparation of competent cells, transformation, M13 manipulation, bacterial media, Western blotting, and ELISA assays were according to Sambrook et al., (Molecular Cloning: A Laboratory Manual, Cold Spring Harbor Laboratory Press, Cold Spring Harbor, N.Y., 1989). The purification of DNA fragments was done using the Geneclean II kit (Bio 101) according to the manufacturer's instructions. SDS gel electrophoresis was performed on a Phastsystem (Pharmacia).

Western blotting was accomplished by transfer of the electrophoresed protein to nitrocellulose using the PhastTransfer system, as described by the manufacturer. The antiserum to SAP was used at a dilution of 1:1000. Horseradish peroxidase labeled anti-IgG was used as the second antibody (see Davis et al., Basic Methods In Molecular Biology, New York, Elsevier Science Publishing Co., pp 1-338, 1986).

B. Isolation of DNA Encoding Saporin

1. Isolation of Genomic DNA and Preparation of Primers for Amplification

Saponaria officinalis leaf genomic DNA was prepared as described in Bianchi et al., Plant Mol. Biol. 11:203-214, 1988. Primers for genomic DNA amplifications were synthesized in a 380B automatic DNA synthesizer. The primer corresponding to the “sense” strand of saporin 57-CTGCAGAATTCGCATGGATCCTGCTTCAAT-3′ (SEQ ID NO. 33) includes an EcoR I restriction site adapter immediately upstream of the DNA codon for amino acid -15 of the native saporin N-terminal leader sequence. The primer 5′-CTGCAGAATTCGCCTCGTTTGACTACTTTG-3′ (SEQ ID NO. 34) corresponds to the “antisense” strand of saporin and complements the coding sequence of saporin starting from the last 5 nucleotides of the DNA encoding the carboxyl end of the mature peptide. Use of this primer introduced a translation stop codon and an EcoRI restriction site after the sequence encoding mature saporin.

2. Amplification of DNA Encoding Saporin

Unfractionated Saponaria officinalis leaf genomic DNA (1 μl) was mixed in a final volume of 100 μl containing 10 mM Tris-HCl (pH 8.3), 50 mM KCl, 0.01% gelatin, 2 mM MgCl₂, 0.2 mM dNTPs, 0.8 μg of each primer. Next, 2.5 U Taq DNA polymerase (Perkin Elmer Cetus) were added and the mixture was overlaid with 30 μl of mineral oil (Sigma). Incubations were done in a DNA Thermal Cycler. One cycle included a denaturation step (94° C. for 1 min), an annealing step (60° C. for 2 min), and an elongation step (72° C. for 3 min). After 30 cycles, a 10 μl aliquot of each reaction was run on a 1.5% agarose gel to verify the structure of the amplified product.

The amplified DNA was digested with EcoRI and subcloned into EcoRI-restricted M13mp18. Single-stranded DNA from recombinant phages was sequenced using oligonucleotides based on internal points in the coding sequence of saporin (see Bennati et al., Eur. J. Biochem. 183.465-470, 1989). Nine of the M13mp18 derivatives were sequenced and compared. Of the nine sequenced clones, five had unique sequences, set forth as SEQ ID NOs. 22 and 35-38, respectively. The clones were designated M13mp18-G4, -G1, -G2, -G7, and -G9. Each of these clones contains all of the saporin coding sequence and 45 nucleotides of DNA encoding the native saporin N-terminal leader peptide.

Saporin DNA sequence was also cloned in the pET11a vector. Briefly, the DNA encoding SAP-6 was amplified by polymerase chain reaction (PCR) from the parental plasmid pZ1B1. The plasmid pZ1B1 contains the DNA sequence for human FGF-2 linked to SAP-6 by a two-amino-acid linker (Ala-Met). PZ1B1 also includes the T7 promoter, lac operator, ribosomal binding site, and T7 terminator present in the pET-11a vector. For SAP-6 DNA amplification, the 5′ primer (5′-CATATGTGTGTCACATCAATCACATTAGAT-3′) (SEQ ID NO. 39), corresponding to the sense strand of SAP-6, incorporated a NdeI restriction enzyme site used for cloning. It also contained a Cys codon at position −1 relative to the start site of the mature protein sequence. No leader sequence was included. The 3′ primer (5′ CAGGTTTGGATCCTTTACGTT 3′) (SEQ ID NO. 40) corresponding to the antisense strand of SAP-6 had a BamHI site used for cloning. The amplified DNA was gel-purified and digested with NdeI and BamHI. The digested SAP-6 DNA fragment was subcloned into the NdeI/BamHI-digested pZ1B1. This digestion removed FGF-2 and the 5′ portion of SAP-6 (up to nucleotide position 650) from the parental rFGF2-SAP vector (pZ1B1) and replaced this portion with a SAP-6 molecule containing a Cys at position −1 relative to the start site of the native mature SAP-6 protein. The resultant plasmid was designated as pZ50B. pZ5OB was transformed into E. coli strain NovaBlue for restriction and sequencing analysis. The appropriate clone was then transformed into E. coli strain BL21(DE3) for expression and large-scale production.

C. Mammalian Codon Optimization of Saporin cDNA.

Mammalian expression plasmids encoding β-galactosidase (β-gal), pSV-β and pNASS-β, were obtained from Clontech (Palo Alto, Calif.). Plasmid pSVβexpresses β-gal from the SV40 early promoter. Plasmid pNASSb is a promoterless mammalian reporter vector containing the β-gal gene.

The amino acid sequence for the plant protein saporin (SAP) was reverse translated using mammalian codons. The resulting mammalian optimized cDNA was divided into 4 fragments (designated 5′-3′ A-D) for synthesis by PCR using overlapping oligos. To facilitate subcloning of each fragment and piecing together of the entire cDNA, restriction enzyme sites were added to the ends of each fragment, and added or removed within each fragment without changing the corresponding amino acid sequence. In addition, the 5′ end of the cDNA was modified to include a Kozak sequence for optimal expression in mammalian cells. Fragments A, B, and D were each synthesized by annealing 4 oligos (2 sense, 2 antisense) with 20 base overlaps and using PCR to fill-in and amplify the fragments. The PCR products were then purified using GeneClean (Bio 101), digested with restriction enzymes recognizing the sites in the primers, and subcloned into pBluescript (SK+) (Stratagene). The sequence of the inserts was verified using Sequenase Version 2.0 (United States Biochemical/Amersham). Fragment C was synthesized in two steps: The 5′ and 3′ halves of the fragment were independently synthesized by PCR using 2 overlapping oligos. The products of these using 2 reactions were then purified and combined and the full-length fragment C was generated by PCR using the outermost oligos as primers. Full-length fragment C was subcloned into pBluescript for sequencing. Fragments A and B were ligated together in pBluescript at an overlapping KspI site. Fragments C and D were ligated together in pBluescript at an overlapping PvuII site. Fragments A-B and C-D were then joined in pBluescript at an overlapping Aval site to give the full-length mammalian optimized SAP cDNA. β-gal sequences were excised from the plasmids pNASS-β and pSV-β(Clontech) by digestion with NotI and replaced with the synthetic SAP gene, which has NotI ends. Orientation of the insert was confirmed by restriction enzyme digestion. Large scale plasmid preparations were performed using Qiagen Maxi 500 columns.

The oligos used to synthesize each SAP fragment are (5′-3′):

| | |
|---|---|
| A1(sense): | |
| CGTATCAGGCGGCCGCCGCCATGGTGACCTCCATCACCCTGGACCTGGTGAACCCCACCGCCGGCC | (SEQ ID NO.41) |
| A2(antisense): | |
| TTGGGGTCCTTCACGTTTGTTGCGGATCTTGTCCACGAAGGAGGAGTACTGGCCGGCGGTGGGGTTCACC | (SEQ ID NO.42) |
| A3(sense): | |
| AACAACGTGAAGGACCCCAACCTGAAGTACGGCGGCACCGACATCGCCGTGATCGGCCCCCCCTC | (SEQ ID NO.43) |
| A4(antisense): | |
| GTGCCGCGGGAGGACTGGAAGTTGATGCGCAGGAACTTCTCCTTGGAGGGGGGGCCGATCACGGC | (SEQ ID NO.44) |
| B1(sense): | |
| CTCCCGCGGCACCGTGTCCCTGGGCCTGAAGCGCGACAACCTGTACGTGGTGGCCTACCTGGCCATGGACAACAC | (SEQ ID NO.45) |
| B2(antisense): | |
| GCGGTCAGCTCGGCGGAGGTGATCTCGGACTTGAAGTAGTAGGCGCGGTTCACGTTGGTGTTGTCCATGGCCAGGTA | (SEQ ID NO.46) |
| B3(sense): | |
| GCCGAGCTGACCGCCCTGTTCCCTGAGGCCACCACCGCCAACCAGAAGGCCCTGGAGTACACCGAGGACTACCAGTCC | (SEQ ID NO.47) |
| B4(antisense): | |
| AGCCCGAGCTCCTTGCGGGACTTGTCGCCCTGGGTGATCTGGGCGTTCTTGTCGATGGACTGGTAGTCCTCGGTGT | (SEQ ID NO.48) |
| C1(sense): | |
| TATAGAATTCCTCGGGCTGGGCATCGACCTGCTGCTGACCTTCATGGAGGCCGTGAACAAGAAGGCCCGCGTGG | (SEQ ID NO.49) |
| C2(antisense): | |
| CGGCGGTCATCTGGATGGCGATCAGCAGGAAGCGGGCCTCGTTCTTCACCACGCGGGCCTTCTTTGTTC | (SEQ ID NO.50) |
| C3(sense): | |
| CGCCATCCAGATGACCGCCGAGGTGGCCCGCTTCCGCTACATCCAGAACCTGGTGACCAAGAACTTCCCC | (SEQ ID NO.51) |
| C4(antisense): | |
| GGCGGATCCCAGCTGACCTCGAACTGGATCACCTTGTTGTCGGAGTCGAACTTGTTGGGGAAGTTCTTGGTCACCA | (SEQ ID NO.52) |
| D1(sense): | |
| CCGGGATCCGTCAGCTGGCGCAAGATCTCCACCGCCATCTACGGCGACGCCAAGAACGGCG | (SEQ ID NO.53) |
| D2(antisense): | |
| GCACCTTGCCGAAGCCGAAGTCGTAGTCCTTGTTGAACACGCCGTTCTTGGCGTCGCCGTAGAT | (SEQ ID NO.54) |
| D3(sense): | |
| TTCGGCTTCGGCAAGGTGCGCCAGGTGAAGGACCTGCAGATGGGCCTGCTGATGTACC | (SEQ ID NO.55) |
| D4(antisense): | |
| TGAACGTGGCGGCCGCCTACTTGGGCTTGCCCAGGTACATCAGCAGGCCCAT | (SEQ ID NO.56) |

D. pOMPAG4 Plasmid Construction

M13 mp18-G4 was digested with EcoR I, and the resulting fragment was ligated into the EcoR I site of the vector pIN-IIIompA2 (see, U.S. Pat. No. 4,575,013 to Inouye; and Duffaud et al., Meth Enz. 153:492-507, 1987) using the methods described herein. The ligation was accomplished such that the DNA encoding saporin, including the N-terminal extension, was fused to the leader peptide segment of the bacterial ompA gene. The resulting plasmid pOMPAG4 contains the lpp promoter (Nakamura et al., Cell 18:1109-1117, 1987), the E coli lac promoter operator sequence (lac O) and the E. coli ompA gene secretion signal in operative association with each other and with the saporin and native N-terminal leader-encoding DNA. The plasmid also includes the E. coli lac repressor gene (lac I).

The M13 mpl8-G1, -G2, -G7, and -G9 clones, respectively, are digested with EcoR I and ligated into EcoR I digested pIN-IIompA2 as described for M13 mp18-G4 above in this example. The resulting plasmids, labeled pOMPAG1, pOMPAG2, pOMPAG7, pOMPA9, are screened, expressed, purified, and characterized as described for the plasmid pOMPAG4.

INV1α competent cells were transformed with pOMPAG4 and cultures containing the desired plasmid structure were grown further in order to obtain a large preparation of isolated pOMPAG4 plasmid using methods described herein.

E. Saporin Expression in E. coli

The pOMPAG4 transformed E. coli cells were grown under conditions in which the expression of the saporin-containing protein is repressed by the lac repressor until the end of the log phase of growth, at which time IPTG was added to induce expression of the saporin-encoding DNA.

To generate a large-batch culture of pOMPAG4 transformed E. coli cells, an overnight culture (approximately 16 hours growth) of JA221 E. coli cells transformed with the plasmid pOMPAG4 in LB broth (see, e.g., Sambrook et al., Molecular Cloning: A Laboratory Manual, Cold Spring Harbor Laboratory Press, Cold Spring Harbor, N.Y., 1989) containing 125 mg/ml ampicillin was diluted 1:100 into a flask containing 750 ml LB broth with 125 mg/ml ampicillin. Cells were grown at logarithmic phase with shaking at 37° C. until the optical density at 550 nm reached 0.9 measured in a spectrophotometer.

In the second step, saporin expression was induced by the addition of IPTG (Sigma) to a final concentration of 0.2 mM. Induced cultures were grown for 2 additional hours and then harvested by centrifugation (25 min., 6500× g). The cell pellet was resuspended in ice cold 1.0 M TRIS, pH 9.0, 2 mM EDTA (10 ml were added to each gram of pellet). The resuspended material was kept on ice for 20-60 minutes and then centrifuged (20 min., 6500× g) to separate the periplasmic fraction of E. coli, which corresponds to the supernatant, from the intracellular fraction corresponding to the pellet.

The E. coli cells containing C-SAP construct in pET11a were grown in a high-cell density fed-batch fermentation with the temperature and pH controlled at 30° C. and 6.9, respectively. A glycerol stock (1 ml) was grown in 50 ml Luria broth until the A₆₀₀ reached 0.6 Inoculum (10 ml) was injected into a 7-1-Applikon (Foster City Calif.) fermentor containing 21 complex batch medium consisting of 5 g/l of glucose, 1.25 g/l each of yeast extract and tryptone (Difco Laboratories), 7 g/l of K₂HPO₄, 8 g/l of KH₂PO₄, 1.66 g/l of (NH₄)₂SO₄, 1 g/l of MgSO₄.7H₂O, 2 ml/l of a trace metal solution (74 g/l of trisodium citrate, 27 g/l of FeCl₃.6H₂O, 2.0 g/l of CoCl₂.6H₂O, 2.0 g/l of Na₂MoO₄.2H₂O, 1.9 g/l of CuSO₄.5H₂0, 1.6 g/l of MnCl₂.4H₂O, 1.4 g/l of ZnCl₂.4H₂O, 1.0 g/l of CaCl₂.2H₂O, 0.5 g/l of H₃BO₃). 2 ml/l of a vitamin solution (6 g/l of thiamin.HCl, 3.05 g/l of niacin, 2.7 g/l of pantothenic acid, 0.7 g/l of pyridoxine.HCl, 0.21 g/l of riboflavin, 0.03 g/l of biotin, 0.02 g/l of folic acid), and 100 mg/l of carbenicillin. The culture was grown for 12 h before initiating the continuous addition of a 40× solution of complex batch media lacking the phosphates and containing only 25 ml/l, each, of trace metal and vitamin solutions. The feed addition continued until the A₆₀₀ of the culture reached 85, at which time (approximately 9 h) the culture was induced with 0.1 mM isopropyl β-D-thiogalactopyranoside. During 4 h of post-induction incubation, the culture was fed with a solution containing 100 g/l of glucose, 100 g/l of yeast extract, and 200 g/l of tryptone. Finally, the cells were harvested by centrifugation (8000× g, 10 min) and frozen at −80° C. until further processed.

The cell pellet (≈400 g wet mass) containing C-SAP was resuspended in 3 vol Buffer B (10 mM sodium phosphate pH 7.0, 5 mM EDTA, S mM EGTA, and 1 mM dithiothreitol). The suspension was passed through a microfluidizer three times at 124 Mpa on ice. The resultant lysate was diluted with NanoPure H₂O until conductivity fell below 2.7 mS/cm. All subsequent procedures were performed at room temperature.

The diluted lysate was loaded onto an expanded bed of Streamline SP cation-exchange resin (300 ml) equilibrated with buffer C (20 mM sodium phosphate pH 7.0, 1 mM EDTA) at 100 ml/min upwards flow. The resin was washed with buffer C until it appeared clear. The plunger was then lowered at 2 cm/min while washing continued at 70 ml/min. Upwards flow was stopped when the plunger was approximately 8 cm away from the bed and the plunger was allowed to move to within 0.5 cm of the packed bed. The resin was firther washed at 70 mllmin downwards flow until A₂₈₀ reached baseline. Buffer C plus 0.25 M NaCl was then used to elute proteins containing C-SAP at the same flow rate.

The eluate was buffer exchanged into buffer D (50 mM sodium borate pH 8.5, 1 mM EDTA) using the Sartocon Mini crossflow filtration system with a 10000 NMolecular Massco module (Sartorius). The sample was then applied to a column of Source 15S (30 ml) equilibrated with buffer D. A 10-column-volume linear gradient of 0-0.3 M NaCl in buffer D was used to elute C-SAP at 30 ml/min.

F. Assay for Cytotoxic Activity

The ribosome inactivating protein activity of recombinant saporin was compared to the ribosome inactivating protein activity of native SAP in an in vitro assay measuring cell-free protein synthesis in a nuclease-treated rabbit reticulocyte lysate (Promega). Samples of immunoaffinity-purified saporin were diluted in PBS and 5 μl of sample was added on ice to 35 μl of rabbit reticulocyte lysate and 10 μl of a reaction mixture containing 0.5 μl of Brome Mosaic Virus RNA, 1 mM amino acid mixture minus leucine, 5 μCi of tritiated leucine and 3 μl of water. Assay tubes were incubated 1 hour in a 30° C. water bath. The reaction was stopped by transferring the tubes to ice and adding 5 μl of the assay mixture, in triplicate, to 75 μl of 1 N sodium hydroxide, 2.5% hydrogen peroxide in the wells of a Millititer HA 96-well filtration plate (Millipore). When the red color had bleached from the samples, 300 μl of ice cold 25% trichloroacetic acid (TCA) were added to each well and the plate left on ice for another 30 min. Vacuum filtration was performed with a Millipore vacuum holder. The wells were washed three times with 300 μl of ice cold 8% TCA. After drying, the filter paper circles were punched out of the 96-well plate and counted by liquid scintillation techniques.

The IC₅₀ for the recombinant and native saporin were approximately 20 pM. Therefore, recombinant saporin-containing protein has full protein synthesis inhibition activity when compared to native saporin.

### Example 2

### Preparation of FGF Muteins

A. Materials and Methods

1. Reagents

Plasmid pFC80, containing the FGF2 coding sequence, was a gift of Drs. Paolo Sarmientos and Antonella Isacchi of Farmitalia Carlo Erba (Milan, Italy). Plasmid pFC80, has been described in the PCT Application Serial No. WO 90/02800 and PCT Application Ser. No. PCT/US93/05702. The sequence of DNA encoding FGF2 in pFC80 is that set forth in PCT Application Serial No. PCT/US93/05702 and in SEQ ID NO. 25.

Plasmid isolation, production of competent cells, transformation and M13 manipulations were carried out according to published procedures (Sambrook et al., Molecular Cloning, a Laboratory Manual, Cold Spring Harbor Laboratory Press, Cold Spring Harbor, N.Y., 1989). Purification of DNA fragments was achieved using the Geneclean II kit, purchased from Bio 101 (La Jolla, Calif.). Sequencing of the different constructions was performed using the Sequenase kit (version 2.0) of USB (Cleveland, Ohio).

2. Sodium Dodecyl Sulphate (SDS) Gel Electrophoresis and Western Blotting

SDS gel electrophoresis was performed on a PhastSystem utilizing 20% gels (Pharmacia). Western blotting was accomplished by transfer of electrophoresed protein to nitrocellulose using the PhastTransfer system (Pharmacia), as described by the manufacturer. The antisera to SAP and basic FGF were used at a dilution of 1:1000. Horseradish peroxidase labeled anti-IgG was used as the second antibody.

B. Preparation of the Mutagenized FGF by Site-directed Mutagenesis

Cysteine to serine substitutions were made by oligonucleotide-directed mutagenesis using the Amersham (Arlington Heights, Ill.) in vitro-mutagenesis system 2.1. Oligonucleotides encoding the new amino acid were synthesized using a 380B automatic DNA synthesizer (Applied Biosystems, Foster City, Calif.).

1. Mutagenesis

The oligonucleotide used for in vitro mutagenesis of cysteine 78 was AGGAGTGTCTGCTAACC (SEQ ID NO. 57), which spans nucleotides 225-241 of FGF2. The oligonucleotide for mutagenesis of cysteine 96 was TTCTAAATCGGTTACCGATGACTG (SEQ ID NO. 58), which spans nucleotides 279-302. The mutated replicative form DNA was transformed into E. coli strain JM109 and single plaques were picked and sequenced for verification of the mutation. The FGF mutated gene was then cut out of M13, ligated into the expression vector pFC80, which had the non-mutated form of the gene removed, and transformed into E. coli strain JM109. Single colonies were picked and the plasmids sequenced to verify the mutation was present. Plasmids with correct mutation were then transformed into the E. coli strain FICE 2 and single colonies from these transformations were used to obtain the mutant basic FGFs. Approximately 20 mg protein per liter of fermentation broth was obtained.

2. Purification of Mutagenized FGF

Cells were grown overnight in 20 ml of LB broth containing 100 μg/ml ampicillin. The next morning the cells were pelleted and transferred to 500 ml of M9 medium with 100 μg/ml ampicillin and grown for 7 hours. The cells were pelleted and resuspended in lysis solution (10 mM TRIS, pH 7.4, 150 mM NaCi, lysozyme, 10 μg/mL, aprotinin, 10 μg/mL, leupeptin, 10 μg/mL, pepstatin A, 10 μg/mL and 1 mM PMSF; 45-60 ml per 16 g of pellet) and incubated while stirring for 1 hour at room temperature. The solution was frozen and thawed three times and sonicated for 2.5 minutes. The suspension was centrifuged; the supernatant saved and the pellet resuspended in another volume of lysis solution without lysozyme, centrifuged again and the supernatants pooled. Extract volumes (40 ml) were diluted to 50 ml with 10 mM TRIS, pH 7.4 (buffer A). Pools were loaded onto a 5 ml Hi-Trap heparin-Sepharose column (Pharmacia, Uppsala, Sweden) equilibrated in 150 mM sodium chloride in buffer A. The column was washed with 0.6 M sodium chloride and 1 M sodium chloride in buffer A and then eluted with 2 M sodium chloride in buffer A. Peak fractions of the 2 M elution, as determined by optical density at 280 nm, were pooled and purity determined by gel electrophoresis. Yields were 10.5 mg of purified protein for the Cys⁷⁸ mutant and 10.9 mg for the Cys⁹⁶ mutant.

The biological activity of [C78S]FGF and [C96S]FGF was measured on adrenal capillary endothelial cells in culture. Cells were plated at 3,000 per well in a 24 well plate in 1 ml of 10% calf serum-HDMEM. Cells were allowed to attach, and samples were added in triplicate at the indicated concentration and incubated for 48 h at 37° C. An equal quantity of samples was added and further incubated for 48 h. Medium was aspirated; cells were treated with trypsin (1 ml volume) to remove cells to 9 ml of Hematall diluent and counted in a Coulter Counter. The results show that the two mutants that retain virtually complete proliferative activity of native basic FGF as judged by the ability to stimulate endothelial cell proliferation in culture.

### Example 3

### Preparation of Mono-derivatized Nucleic Acid Binding Domain (MYOD)

MyoD at a concentration of 4.1 mg/ml is dialyzed against 0.1 M sodium phosphate, 0.1 M sodium chloride, pH 7.5. A 1.1 molar excess (563 μg in 156 μl of anhydrous ethanol) of SPDP (Pharmacia, Uppsala, Sweden) is added and the reaction mixture immediately agitated and put on a rocker platform for 30 minutes. The solution is then dialyzed against the same buffer. An aliquot of the dialyzed solution is examined for extent of derivatization according to the Pharmacia instruction sheet. The extent of derivatization is typically 0.79 to 0.86 moles of SPDP per mole of nucleic acid binding domain.

Derivatized myoD (32.3 mg) is dialyzed in 0.1 M sodium borate, pH 9.0 and applied to a Mono S 16/10 column equilibrated with 25 mM sodium chloride in dialysis buffer. A gradient of 25 mM to 125 mM sodium chloride in dialysis buffer elutes free and derivatized nucleic acid binding domain. The flow rate is 4.0 ml/min, 4 ml fractions are collected. Aliquots of fractions were assayed for protein concentration (BCA Protein Assay, Pierce Chemical, Chicago, Ill.) and for pyridylthione released by reducing agent. Individual fractions (25 to 37) are analyzed for protein concentration and pyridyl-disulfide concentration. The data indicate a separation according to the level of derivatization by SPDP. The initial eluting peak is composed of myoD that is approximately di-derivatized; the second peak is mono-derivatized and the third peak shows no derivatization. The di-derivatized material accounts for approximately 20% of the three peaks; the second accounts for approximately 48% and the third peak contains approximately 32%. Material from the second peak is pooled and gives an average ratio of pyridyl-disulfide to myoD of 0.95. Fraction 33, which showed a divergent ratio of pyridine-2-thione to +protein, was excluded from the pool. Fractions that showed a ratio of SPDP to myoD greater than 0.85 but less than 1.05 are pooled, dialyzed against 0.1 M sodium chloride, 0.1 M sodium phosphate, pH 7.5 and used for derivatization with basic FGF.

### Example 4

### Preparation of Modified Nucleic Acid Binding Domain (MyoD)

As an alternative to derivatization, myoD is modified by addition of a cysteine residue at or near the N-terminus-encoding portion of the DNA. The resulting myoD can then react with an available cysteine on an FGF or react with a linker or a linker attached to an FGF to produce conjugates that are linked via the added Cys.

Modified myoD is prepared by modifying DNA encoding the myoD (GenBank Accession No. X56677). DNA encoding Cys is inserted at position −1 or at a codon within 10 or fewer residues of the N-terminus. The resulting DNA is inserted into pET11a and pET15b and expressed in BL21 cells (NOVAGEN, Madison, Wis.).

A. Preparation of myoD with an Added Cysteine Residue at the N-terminus

Primer #1 corresponding to the sense strand of myoD, nucleotides 121-144, incorporates a NdeI site and adds a Cys codon 5′ to the start site for the mature protein

| | |
|---|---|
| 5′-CATATGTGTGAGCTACTGTCGCCACCGCTC-3′ | (SEQ ID NO.59) |

Primer #2 is an antisense primer complementing the coding sequence of nucleic acid binding domain spanning nucleotides 1054-1077 and contains a BamHI site.

| | |
|---|---|
| 5′-GGATCCGAGCACCTGGTATATCGGTGGGGG-3′ | (SEQ ID NO.60) |

MyoD DNA is amplified by PCR as follows using the above primers. A clone containing a full4ength DNA (or cDNA) for myoD (1 μl) is mixed in a final volume of 100 μl containing 10 mM Tris-HCl (pH 8.3), 50 mM KCl, 0.01% gelatin, 2 mM MgCl₂, 0.2 mM dNTPs, 0.8 μg of each primer. Next, 2.5 U TaqI DNA polymerase (Boehringer Mannheim) is added and the mixture is overlaid with 30 μl of mineral oil (Sigma). Incubations are done in a DNA Thermal Cycler. Cycles include a denaturation step (94° C. for 1 min), an annealing step (60° C. for 2 min), and an elongation step (72° C. for 3 min). After 35 cycles, a 10 μl aliquot of each reaction is run on a 1.5% agarose gel to verify the correct structure of the amplified product.

The amplified DNA is gel purified and digested with NdeI and BamHI and subcloned into NdeI and BamHI-digested plasmid containing FGF/myoD. This digestion and subcloning step removes the FGF-encoding DNA and 5′ portion of SAP up to the BamHI site at nucleotides 555-560 and replaces this portion with DNA encoding a myoD molecule that contains a cysteine residue at position −1 relative to the start site of the native mature SAP protein.

B. Preparation of NABD with a Cysteine Residue at Position 4 or 10

These constructs are designed to introduce a cysteine residue at position 4 or 10 of the native protein by replacing the Ser residue at position 4 or the Val residue at position 10 with cysteine.

MyoD is amplified by polymerase chain reaction (PCR) from the parental plasmid encoding the FGF-nucleic acid binding domain fusion protein using primers that incorporate a TGT or TGC codon at position 4 or 10.

The PCR conditions are performed as described above, using the following cycles: denaturation step 94° C. for 1 minute, annealing for 2 minutes at 60° C., and extension for 2 minutes at 72° C. for 35 cycles. The amplified DNA is gel purified, digested with NdeI and BamHI, and subcloned into NdeI and BamHI digested pET11a This digestion removes the FGF and 5′ portion of nucleic acid binding domain (up to the newly added BamHI) from the parental FGF- myoD vector and replaces this portion with a myoD molecule containing a Cys at position 4 or 10 relative to the start site of the native protein.

The resulting plasmid is digested with NdeI/BamHI and inserted into pETI Sb (NOVAGEN, Madison, Wis.), which has a His-Tag™ leader sequence that has also been digested NdeI/BamHI.

DNA encoding unmodified myoD can be similarly inserted into a pET5b or pET11A and expressed as described below for the modified SAP-encoding DNA.

C. Expression of the Modified Nucleic Acid Binding Domain-encoding DNA

BL21(DE3) cells are transformed with the resulting plasmids and cultured as described in Example 2, except that all incubations were conducted at 30° C. instead of 37° C. Briefly, a single colony is grown in LB AMP₁₀₀ to and OD₆₀₀ of 1.0-1.5 and then induced with IPTG (final concentration 0.1 mM) for 2 h. The bacteria are spun down.

D. Purification of Modified Nucleic Acid Binding Domain

Lysis buffer (20 mM NaPO₄, pH 7.0, 5 mM EDTA, 5 mM EGTA, 1 mM DTT, 0.5 μg/ml leupeptin, 1 μg/ml aprotinin, 0.7 μg/ml pepstatin) was added to the myoD cell paste (produced from pZ50B1 in BL21 cells, as described above) in a ratio of 1.5 ml buffer/g cells. This mixture is evenly suspended via a Polytron homogenizer and passed through a microfluidizer twice.

The resulting lysate is centrifuged at 50,000 rpm for 45 min. The supernatant is diluted with SP Buffer A (20 mM NaPO₄, 1 mM EDTA, pH 7.0) so that the conductivity is below 2.5 mS/cm. The diluted lysate supernatant is then loaded onto a SP-Sepharose column, and a linear gradient of 0 to 30% SP Buffer B (1 M NaCl, 20 mM NaPO₄, 1 mM EDTA, pH 7.0) in SP Buffer A with a total of 6 column volumes is applied. Fractions containing myoD are combined and the resulting Nucleic acid binding domain had a purity of greater than 90%. A buffer exchange step is used to get the SP eluate into a buffer containing 50 mM NaBO₃, 1 mM EDTA, pH 8.5 (S Buffer A). This sample is then applied to a Resource S column (Pharmacia, Sweden) pre-equilibrated with S Buffer A. Pure nucleic acid binding domain is eluted off the column by 10 column volumes of a linear gradient of 0 to 300 mM NaCl in SP Buffer A.

In this preparation, ultracentrifugation is used clarify the lysate; other methods, such as filtration and using floculents also can be used. In addition, Streamline S (PHARMACIA, Sweden) may also be used for large scale preparations.

### Example 5

### Preparation of Chemical Conjugates Containing FGF Muteins

[C78S]FGF or [C96S]FGF (1 mg; 56 nmol) that had been dialyzed against phosphate-buffered saline is added to 2.5 mg mono-derivatized nucleic acid binding domain (a 1.5 molar excess over the basic FGF mutants) and left on a rocker platform overnight. The next morning the ultraviolet-visible wavelength spectrum is taken to determine the extent of reaction by the release of pyridylthione, which adsorbs at 343 nm with a known extinction coefficient The ratio of pyridylthione to basic FGF mutant for [C78S]FGF is 1.05 and for [C96S]FGF is 0.92. The reaction mixtures are treated identically for purification in the following manner: reaction mixture is passed over a HiTrap heparin-Sepharose column (1 ml) equilibrated with 0.15 M sodium chloride in buffer A at a flow rate of 0.5 ml/min. The column is washed with 0.6 M NaCl and 1.0 M NaCl in buffer A and the product eluted with 2.0 M NaCl in buffer A. Fractions (0.5 ml) are analyzed by gel electrophoresis and absorbance at 280 μm. Peak tubes are pooled and dialyzed versus 10 mM sodium phosphate, pH 7.5 and applied to a Mono-S 5/5 column equilibrated with the same buffer. A 10 ml gradient between 0 and 1.0 M sodium chloride in equilibration buffer is used to elute the product. Purity is determined by gel electrophoresis and peak fractions were pooled.

Under these conditions, virtually 100% of the mutant FGFs reacts with mono-derivatized myoD. Because the free surface cysteine of each mutant acts as a free sulfhydryl, it is unnecessary to reduce cysteines after purification from the bacteria. The resulting product is purified by heparin-Sepharose (data not shown), thus establishing that heparin binding activity of the conjugate is retained.

### Example 6

### Recombinant Production of FGF-nucleic Acid Binding Domain Fusion Protein

A. General Descriptions

1. Bacterial Strains and Plasmids

E. coli strains BL21(DE3), BL21(DE3)pLysS, HMS174(DE3) and HMS174(DE3)pLysS were purchased from NOVAGEN, Madison, Wis. Plasmid pFC80, described below, has been described in the WIPO International Patent Application No. WO 90/02800, except that the bFGF coding sequence in the plasmid designated pFC80 herein has the sequence set forth as SEQ ID NO. 25, nucleotides 1-465. The plasmids described herein may be prepared using pFC80 as a starting material or, alternatively, by starting with a fragment containing the clI ribosome binding site (SEQ ID NO. 61) linked to the FGF-encoding DNA.

E. coli strain JA221 (lpp⁻ hdsM+ trpE5 leuB6 lacY recA1 F′[lacI^{q} lac⁺ pro⁺]) is publicly available from the American Type Culture Collection (ATCC), Rockville, Md. 20852, under the accession number ATCC 33875. (JA221 is also available from the Northern Regional Research Center (NRRL), Agricultural Research Service, U.S. Department of Agriculture, Peoria, Ill. 61604, under the accession number NRRL B-15211; see also U.S. Pat. No. 4,757,013 to Inouye; and Nakamura et al., Cell 18:1109-1117, 1979). Strain INV1α is commercially available from Invitrogen, San Diego, Calif.

B. Construction of Plasmids Encoding FGF/NABD Fusion Proteins

1. Construction of FGFM13 with a cI Ribosome Binding Site Linked to FGF

A Nco I restriction site is introduced into the nucleic acid binding domain-encoding DNA by site-directed mutagenesis using the Amersham in vitro-mutagenesis system 2.1. The oligonucleotide employed to create the Nco I restriction site is synthesized using a 380B automatic DNA synthesizer (Applied Biosystems). This oligonucleotide containing the Nco I site replaces the original nucleic acid binding domain-containing coding sequence.

In order to produce a bFGF coding sequence in which the stop codon was removed, the FGF-encoding DNA is subcloned into a M13 phage and subjected to site-directed mutagenesis. Plasmid pFC80 is a derivative of pDS20 (see, e.g., Duester et al., Cell 30:855-864, 1982; see also U.S. Pat. Nos. 4,914,027, 5,037,744, 5,100,784, and 5,187,261; see also PCT International Application No. WO 90/02800; and European Patent Application No. EP 267703 Al), which is almost the same as plasmid pKG1800 (see Bernardi et al., DNA Sequence 1:147-150, 1990; see also McKenney et al. (1981) pp. 383-415 in Gene Amplification and Analysis 2: Analysis of Nucleic Acids by Enzymatic Methods, Chirikjian et al. (eds.), North Holland Publishing Company, Amsterdam) except that it contains an extra 440 bp at the distal end of galK between nucleotides 2440 and 2880 in pDS20. Plasmid pKG1800 includes the 2880 bp EcoR I-Pvu II of pBR322 that contains the contains the ampicillin resistance gene and an origin of replication.

Plasmid pFC80 is prepared from pDS20 by replacing the entire galK gene with the FGF-encoding DNA of SEQ ID NO. 25, inserting the trp promoter (SEQ ID NO. 62) and the bacteriophage lambda cII ribosome binding site (SEQ. ID NO. 61; see, e.g., Schwarz et al., Nature 272:410, 1978) upstream of and operatively linked to the FGF-encoding DNA. The Trp promoter can be obtained from plasmid pDR720 (Pharmacia PL Biochemicals) or synthesized. Plasmid pFC80, contains the 2880 bp EcoR I-BamH I fragment of plasmid pSD20, a synthetic Sal I-Nde I fragment that encodes the Trp promoter region:

| | |
|---|---|
| EcoR I | |
| AATTCCCCTGTTGACAATTAATCATCGAACTAGTTAACTAGTACGCAGCTTGGCTGCAG | (SEQ ID NO.62) |
| and the cII ribosome binding site): | |
| Sal I Nde I | |
| GTCGACCAAGCTTGGGCATACATTCAATCAATTGTTATCTAAGGAAATACTTCATATG | (SEQ ID NO.61) |

The FGF-encoding DNA is removed from pFC80 by treating it as follows. The pFC80 plasmid was digested by Hga I and SalI, which produces a fragment containing the CII ribosome binding site linked to the FGF-encoding DNA. The resulting fragment is blunt ended with Klenow's reagent and inserted into M13mp 18 that has been opened by Sma I and treated with alkaline phosphatase for blunt-end ligation. In order to remove the stop codon, an insert in the ORI minus direction is mutagenized using the Amersham kit, as described above, using the following oligonucleotide (SEQ ID NO. 63): GCTAAGAGCGCCATGGAGA, which contains one nucleotide between the FGF carboxy terminal serine codon and a Nco I restriction site; it replaces the following wild type FGF encoding DNA having SEQ ID NO. 64:

GCT AAG AGC TGA CCA TGG AGA
Ala Lys Ser STOP Pro Trp Arg

The resulting mutant derivative of M13mpl8, lacking a native stop codon after the carboxy terminal serine codon of bFGF, was designated FGFM 13. The mutagenized region of FGFM 13 contained the correct sequence.

2. Preparation of a Plasmid That Encodes the FGF/MyoD Fusion Protein

Plasmid FGFM13 is cut with Nco I and Sac I to yield a fragment containing the CII ribosome binding site linked to the bFGF coding sequence with the stop codon replaced.

An M13mp18 derivative containing the myoD coding sequence is also cut with restriction endonucleases Nco I and Sac I, and the bFGF coding fragment from FGFM13 was inserted by ligation to DNA encoding the fusion protein bFGF- myoD into the M13mp18 derivative to produce mpFGF- myoD, which contains the CII ribosome binding site linked to the FGF-nucleic acid binding domain fusion gene.

Plasmid mpFGF-myoD is digested with Xba I and EcoR I and the resulting fragment containing the bFGF- myoD coding sequence is isolated and ligated into plasmid pET-11a (available from NOVAGEN, Madison, Wis.; for a description of the plasmids see U.S. Pat. No. 4,952,496; see also Studier et al., Meth. Enz. 185:60-89, 1990; Studier et al., J. Mol. Biol. 189:113-130, 1986; Rosenberg et al., Gene 56:125-135, 1987) that has also been treated with EcoR I and Xha I.

E. coli strain BL21(DE3)pLysS (NOVAGEN, Madison WI) may be transformed with the plasmid containing the fusion gene.

Plasmid FGF/myoD may be digested with EcoR I, the ends repaired by adding nucleoside triphosphates and Klenow DNA polymerase, and then digested with Nde I to release the FGF-encoding DNA without the CII ribosome binding site. This fragment is ligated into pET 11a, which is BamH I digested, treated to repair the ends, and digested with Nde I. The resulting plasmid includes the T7 transcription terminator and the pET-11a ribosome binding site.

Plasmid FGF/myoD may be digested with EcoR I and Nde I to release the FGF-encoding DNA without the CII ribosome binding site and ends are repaired as described above. This fragment may be ligated into pET 12a, which had been BamH I digested and treated to repair the ends. The resulting plasmid includes DNA encoding the OMP T secretion signal operatively linked to DNA encoding the fusion protein.

3. Preparation of a Plasmid That Encodes FGF2-protamine Fusion Protein

Protamines are small basic DNA binding proteins, approximately 6.8 kD in molecular weight with a isoelectric point of 12.175. Twenty-four of the fifty one amino acids are strongly basic. Human protamine has been shown to condense genomic DNA for packaging into the sperm head. The positive charges of the protamine react with the negative charges of the phosphate backbone of the DNA.

A FGF-protamine fusion protein that has the ability to bind to the FGF receptor and bind DNA with high affinity is constructed for expression in E coli. The sequence for the human protamine gene is obtained from GenBank (accession no. Y00443). Four overlapping oligonucleotides (60mers) are generated and used to amplify the protamine gene. The amplified product is purified and ligated into the bacterial expression vector pET11 a (Novagen). To facilitate subcloning, a Ncol and BamHI site are incorporated into the primers. The fragment is synthesized by annealing the 4 oligos (2 sense and 2 antisense) with 20 base overlaps and using PCR to fill-in and amplify the fragments. The PCR products are digested with NcoI and BamHI, and subcloned into pBluescript SK+. The insert sequence is verified. The sequenced product is then cloned downstream and in-frame with FGF2, which has been previously cloned into the pET11a expression plasmid. The oligos used to generate fragment A are (5′-3′):

| | |
|---|---|
| PT1: | |
| TACATGCCATGGCCAGGTACAGATGCTGTCGCAGCCAGAGCCGGAGCAGATATFACCGCC | (SEQ ID NO.65) |
| PT2: | |
| GCAGCTCCGCCTCCTTCGTCTGCGACTTCTTTGTCTCTGGCGGTAATATCTGCTCCGGCT | (SEQ ID NO.66) |
| PT3: | |
| GACGAAGGAGGCGGAGCTGCCAGACACGGAGGAGAGCCATGAGGTGCTGCCGCCCCAGGT | (SEQ ID NO.67) |
| PT4: | |
| ATATATCCTAGGTTAGTGTCTTCTACATCTCGGTCTGTACCTGGGGCGGCAGCACCTCA | (SEQ ID NO.68) |

Competent bacterial cells, BL21 (DE3), are transformed with the pET11-FGF2-protamine construct. The cells are initially plated on LB agar plates containing 100 μg/ml ampicillin. A glycerol stock made from an individual colony added to 1 ml fresh LB broth and then to 250 ml of LB broth. The cells are grown to an OD₆₀₀ of 0.7 and induced with IPTG. The culture is harvested 4 hours after induction. The suspension is centrifuged; the supernatant is saved and the pellet is resuspended in lysis buffer, centrifuged again and the supernatants pooled. A sample of the pellet and the supernatant are analyzed by Western analysis using antibodies to FGF2 to determine the percentage of fusion protein within each fraction. Soluble protein is purified. Briefly, the cells are pelleted and resuspended in buffer A (10 mM sodium phosphate, pH 6.0, containing 10 mM EDTA, 10 mM EGTA and 50 mM NaCl) and passed through a microfluidizer (Microfluidics Corp., Newton, Mass.) to break open the bacteria and shear DNA. The resultant mixture is diluted and loaded onto an expanded bed Streamline SP cation-exchange resin. The column is washed with step gradients of increasing concentrations of NaCl. The eluted material is analyzed by Western analysis for fractions containing the fusion protein. These fractions are pooled, diluted, and loaded onto a Heparin-Sepharose affinity column. After washing, the bound proteins are eluted in a batch-wise manner in buffer containing 1 M NaCl and then in buffer containing 2 M NaCl. Peak fractions of the 2M elution, as determined by optical density at 280 nm, are pooled and the purity determined by gel electrophoresis and Western analysis. The final pool of material will be loaded onto a column of Sephacryl S-100 equilibrated with 20 mM HEPES pH 7.4, 150 mM NaCl.

Fusion protein located in the pellet is isolated, solubilized and refolded. Briefly, each culture pellet is thawed completely and resuspended in buffer A (10 mM Tris, 1 mM EDTA, pH 8.0+0.1 mg/ml lyzozyme). The mixture is sonicated on ice, centrifuged at 16,000× g, and the supernatant discarded. Inclusion bodies are solubilized with solubilization buffer: (6 M guanidine-HCl, 100 mM Tris, 150 mM NaCl, 50 mM EDTA, 50 mM EGTA, pH 9.5,), vortexed, incubated for 30 minutes at room temperature, and centrifuged at 35,000× g for 15 minutes. The supernatant is saved and diluted 1:10 in dilution buffer (100 mM Tris, 10 mM EDTA, 1% monothioglycerol, 0.25 M L-arginine, pH 9.5). The material is stirred, covered, at 4° C. for 2 hours and then centrifuged at 35,000 X g for 20 minutes. The supernatant is dialyzed in against 5 liters PBS, pH 8.8, for 24 hours at 4° C. with 3 changes of fresh PBS. The material is concentrated approximately 10-fold using size-exclusion spin columns. The soluble refolded material is then analyzed by gel electrophoresis.

Expression of the FGF-protarnine fusion protein can be achieved in mammalian cells by excising the insert with restriction enzymes NdeI and BamHI and ligating into a mammalian expression vector.

C. Expression of the Recombinant bFGF-NABD Fusion Proteins

A two-stage method is used to produce recombinant bFGF-myoD protein (hereinafter bFGF-nucleic acid binding domain fusion protein).

Three liters of LB broth containing ampicillin (50 μg/ml) are inoculated with plasmid-containing bacterial cells (strain BL21(DE3)pLysS) from an overnight culture (1:100 dilution). Cells are grown at 30° C. in an incubator shaker to an OD₆₀₀ of 1.5. IPTG (Sigma Chemical, St. Louis, Mo.) is added to a final concentration of 0.2 mM and growth was continued for 2 to 2.5 hours at which time cells were centrifuged.

The pellet is resuspended in lysis solution (45-60 ml per 16 g of pellet; 20 mM TRIS, pH 7.4, 5 mM EDTA, 10% sucrose, 150 mM NaCl, lysozyme, 100 μg/ml, aprotinin, 10 μg/ml, leupeptin, 10 μg/ml, pepstatin A, 10 μg/ml and 1 mM PMSF) and incubated with stirring for 1 hour at room temperature. The solution is sonicated for 2.5 minutes. The suspension is centrifuged at 12,000 X g for 1 hour; the resulting frst-supernatant saved and the pellet is resuspended in another volume of lysis solution without lysozyrne. The resuspended material is centrifuged again to produce a second-supernatant, and the two supernatants are pooled and dialyzed against borate buffered saline, pH 8.3.

D. Affinity Purification of bFGF-NABD Fusion Protein

Thirty ml of the dialyzed solution containing the FGF2-nucleic acid binding domain fusion protein is applied to HiTrap heparin-Sepharose column (Pharmacia, Uppsala, Sweden) equilibrated with 0.15 M NaCl in 10 mM TRIS, pH 7.4 (buffer A). The column is washed first with equilibration buffer; second with 0.6 M NaCl in buffer A; third with 1.0 M NaCl in buffer A; and finally eluted with 2 M NaCl in buffer A into 1.0 ml fractions. Samples were assayed by the ELISA method.

bFGF-nucleic acid binding domain fusion protein binds the heparin-Sepharose column at similar affinity as native and recombinantly-produced bFGF, indicating that the heparin affinity is retained in the bFGF-SAP fusion protein.

E. Characterization of the bFGF-NABD Fusion Protein by Western Blot

SDS gel electrophoresis is performed on a Phastsystem utilizing 20% acrylamide gels (Pharmacia). Western blotting is accomplished by transfer of the electrophoresed protein to nitrocellulose using the PhastTransfer system (Pharmacia), as described by the manufacturer. Antisera to bFGF is used at a dilution of 1:1000. Horseradish peroxidase labeled anti-IgG is used as the second antibody (Davis et al., Basic Methods in Molecular Biology, New York, Elsevier Science Publishing Co., pp 1-338, 1986).

Anti-FGF antisera should bind to a protein with an approximate molecular weight of 53,000, which corresponds to the sum of the independent molecular weights of nucleic acid binding domain (35,000) and bFGF (18,000).

### Example 7

### Preparation OF FGF-nucleic Acid Binding Domain Conjugates That Contain Linkers Encoding Protease Substrates

A. Synthesis of Oligos Encoding Protease Substrates

Complementary single-stranded oligos in which the sense strand encodes a protease substrate, have been synthesized either using a cyclone machine (Millipore, Mass.) according the instructions provided by the manufacturer, or were made by Midland Certified Reagent Co. (Midland, Tex.) or by National Biosciences, Inc. (Minn.). The following oligos have been synthesized.

| | |
|---|---|
| 1. Cathepsin B substrate linker | |
| 5′-CCATGGCCCTGGCCCTGGCCCTGGCCCTGGCCATGG | SEQ ID |
| | NO:69 |
| 2. Cathepsin D substrate linker | |
| 5′-CCATGGGCCGATCGGGCTTCCTGGGCTTCGGCTTCCTGG | SEQ ID |
| GCTTCGCCAT GG -3′ | NO:70 |
| 3. Trypsin substrate linker | |
| 5′-CCATGGGCCGATCGGGCGGTGGGTGCGCTGGTAATAGAGT | SEQ ID |
| CAGAAGATCAGTCGGAAGCAGCCTGTCTTGCGGTGGTCTC | NO:71 |
| GACCTGCAGG CCATGG-3′ | |
| 4. Gly₄Ser | |
| 5′-CCATGGGCGG CGGCGGCTCT GCCATGG-3′ | SEQ ID |
| | NO:26 |
| 5. (Gly₄Ser)₂ | |
| 5′-CCATGGGCGGCGGCGGCTCTGGCGGCGGCGGCTC | SEQ ID |
| TGCCATGG-3′ | NO:27 |
| 6. (Ser₄Gly)₄ | |
| 5′-CCATGGCCTCGTCGTCGTCGGGCTCGTCGTCGTC | SEQ ID |
| GGGCTCGTCGTCGTCGGGCTCGTCGTCGTCGGGC | NO:28 |
| GCCATGG-3′ | |
| 7. (Ser₄Gly)₂ | |
| 5-CCATGGCCTCGTCGTCGTCGGGCTCGTCGTCGTC | SEQ ID |
| GGGCGCCATGG-3′ | NO:29 |
| 8. Thrombin substrate linker | |
| CTG GTG CCG CGC GGC AGC | SEQ ID |
| Leu Val Pro Arg Gly Ser | NO.72 |
| 9. Enterokinase substrate linker | |
| GAC GAC GAC GAC CCA | SEQ ID |
| Asp Asp Asp Asp Lys | NO.73 |
| 10. Factor Xa substrate | |
| ATC GAA GGT CGT | SEQ ID |
| Ile Glu Gly Arg | NO.74 |

### B. Preparation of DNA Constructs Encoding FGF-Linker-NABD

The complementary oligos are annealed by heating at 95° C. for 15 min., cooled to room temperature, and then incubated at 4° C. for a minute to about an hour. Following incubation, the oligos are digested with NcoI and ligated overnight at a 3:1 (insert:vector) ratio at 15° C. to NcoI-digested plasmid which has been treated with alkaline phosphatase (Boehringer Mannheim).

Bacteria (Novablue (NOVAGEN, Madison, Wis.)) are transformed with the ligation mixture (1 μl) and plated on LB-amp or LB-Kan, depending upon the plasmid). Colonies are selected, clones isolated and sequenced to determine orientation of the insert. Clones with correct orientation are used to transform strain expression strain BL21(DE3) (NOVAGEN, Madison, Wis.). Glycerol stocks are generated from single transformed colonies. The transformed strains are cultured as described in Example 2 and fusion proteins with linkers were expressed.

The DNA and amino acid sequences of exemplary fusion proteins, containing cathepsin B substrate (FPFS9), cathepsin D substrate (FPFS5), Gly₄Ser (FPFS7), (Gly₄Ser)₂ (FPFSS), trypsin substrate (FPFS6), (Ser₄Gly)₄ (FPFS12) and (Ser₄Gly)₂ (FPFS11) linkers, respectively, are set forth in SEQ ID NOs. 75-81.

### Example 8

### FGF-poly-L-lysine (FGF2-K) Complexed with a Plasmid Encoding β-galactosidase

A. Derivatization of poly-L-lysine

Polylysine polymer with average lengths of 13, 39, 89, 152, and 265 (K₁₃, K₃₉, K₈₄, K152, K₂₆₅) are purchased from a commercial vendor (Sigma, St. Louis, Mo.) and dissolved in 0.1 M NaPO₄, 0.1 M NaCl, 1 mM EDTA, pH 7.5 (buffer A) at 3-5 mg/ml. Approximately 30 mg of poly-L-lysine solution is mixed with 0.1 87 ml of 3 mg/ml N-succinimidyl-3(pyridyldithio)proprionate (SPDP) in anhydrous ethanol resulting in a molar ratio of SPDP/poly-L-lysine of 1.5 and incubated at room temperature for 30 minutes. The reaction mixture is then dialyzed against 4 liters of buffer A for 4 hours at room temperature.

B. Conjugation of derivatized polylysine to FGF2-3

A solution containing 28.5 mg of poly-L-lysine-SPDP is added to 12.9 mg of FGF2-3 ([C96S]-FGF2) in buffer A and incubated overnight at 4° C. The molar ratio of poly-L-lysine-SPDP/FGF2-3 is approximately 1.5. Following incubation, the conjugation reaction mixture is applied to a 6 ml Resource S (Pharmacia, Uppsala, Sweden) column. A gradient of 0.15 M to 2.1 M NaCl in 20 mM NaPO₄, 1 mM EDTA, pH 8.0 (Buffer B) over 24 column volumes is used for elution. The FGF2-3/poly-L-lysine conjugate, called FGF2-K, is eluted off the column at approximately 1.8-2 M NaCl concentration. Unreacted FGF2-3 is eluted off by 0.5-0.6 M NaCl.

The fractions containing FGF2-K are concentrated and loaded onto a gel-filtration column (Sephacryl S100) for buffer exchange into 20 mM HEPES, 0.1 M NaCl, pH 7.3. The molecular weight of FGF-K152 as determined by size exclusion HPLC is approximately 42 kD. To determine if the conjugation procedure interferes with the ability of FGF2-3 to bind heparin, the chemical conjugate FGF2-K is loaded onto a heparin column and eluted off the column at 1.8-2.0 M NaCl. In comparison, unconjugated FGF2-3 is eluted off heparin at 1.4-1.6 M NaCl. This suggests that poly-L-lysine contributes to FGF2-3 ability to bind heparin. The ability of poly-L-lysine 152 to bind heparin is not determined; poly-L-lysine 84 elutes at approximately 1.6 M NaCl. Histone HI-polylysine was purchased and cytochrome C was conjugated to polylysine as described herein.

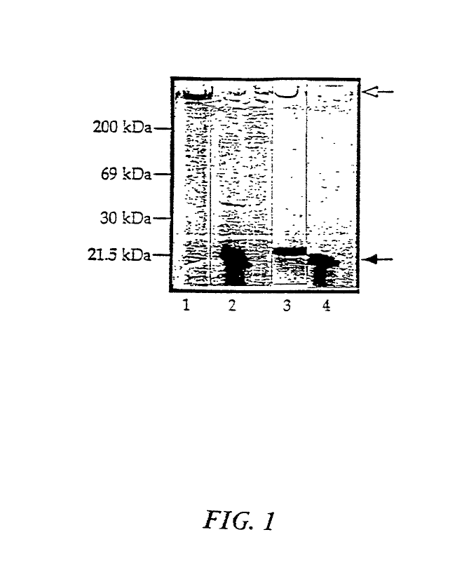

A sample of FGF2-K is electrophoresed on SDS-PAGE under non-reducing and reducing conditions. The protein migrates at the same molecular weight as FGF. Under non-reducing conditions the conjugate does not enter the gel because of its high charge density (FIG. 1, lanes 1, 2, non-reducing; lanes 3, 4, reducing).

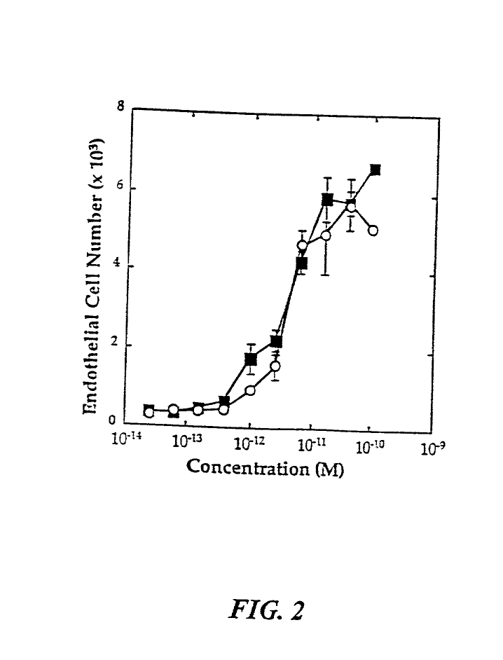

A standard proliferation assay using aortic bovine endothelial cells is performed to determine if the conjugation procedure reduced the ability of FGF2-3 ability to stimulate mitogenesis. The results reveal that FGF2-K is equivalent to FGF2-3 in stimulating proliferation (FIG. 2).

C. FGF2-3-poly-L-lysine-nucleic Acid Complex Formation

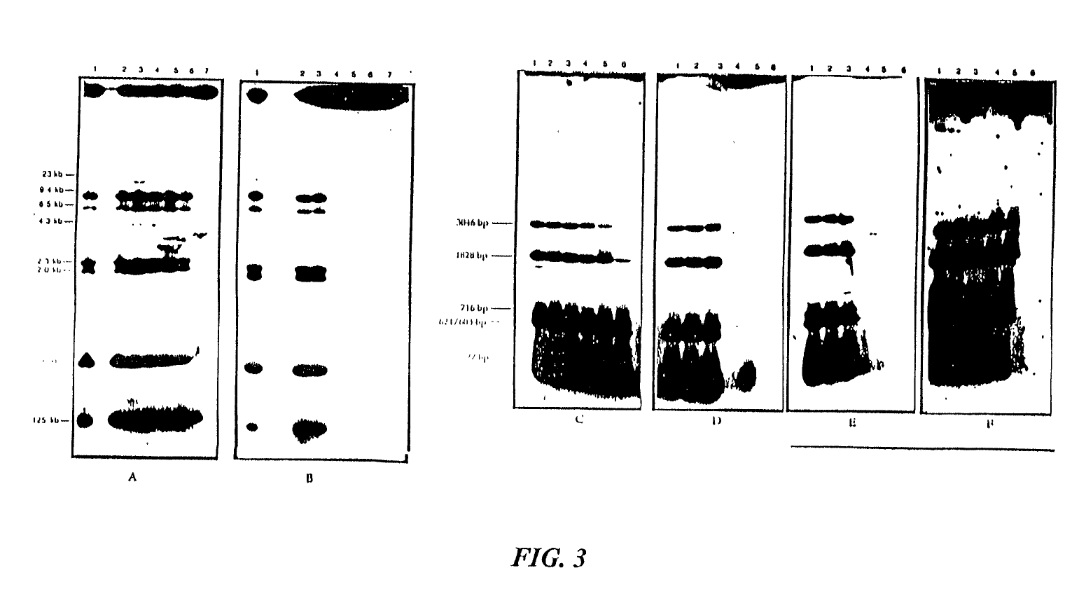

Optimal conditions for complex formation are established. Varying quantities (0.2 to 200 μg) of β-galactosidase encoding plasmid nucleic acid pSVβ or pNASS-β (lacking a promoter) are slowly mixed with 100 μg of FGF2-K in 20 mM HEPES pH 7.3, 0.15 M NaCl. The reaction is incubated for 1 hour at room temperature. Nucleic acid binding to the FGF-lysine conjugate is confirmed by gel mobility shift assay using ³²P-labeled SV40-β-gal nucleic acid cut with HincII restriction endonuclease. In brief, SV40β-gal nucleic acid is digested with HincII restriction endonucleases; ends are labeled by T₄ PNK following dephosphorylation with calf intestinal alkaline phosphatase. To each sample of 35 ng of ³²P-labeled nucleic acid increasing amounts of FGF-polylysine conjugate is added to the mixture. The protein/nucleic acid mixture is electrophoresed in an agarose gel with 1× TAE buffer. Binding of the conjugate to the radiolabeled DNA is shown by a shift in the complex to the top of the well. (FIG. 3.) As seen in FIG. 3D, as little as 10 ng of K₈₄ causes a complete shift of restriction fragments indicating binding. With K₁₃, 100 ng of poly-L-lysine was required (FIG. 3C). With K₂₆₅, 10 ng was required (FIG. 3E).

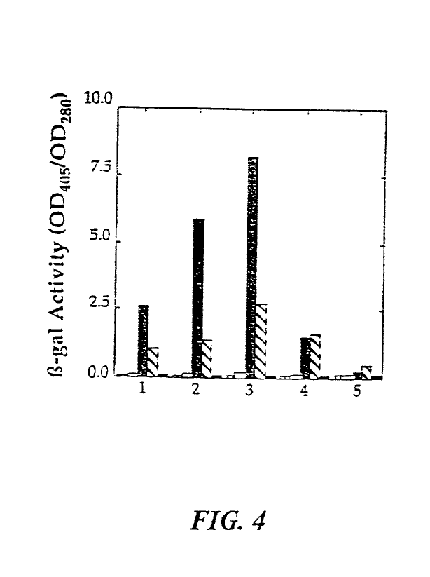

The optimal length of poly-L-lysine and weight ratios is determined by conjugation of FGF2-3 to poly-lysine of different lengths. DNA encoding β-galactosidase was complexed with the conjugates at 10:1, 5:1, 2:1, 1:1, and 0.5:1 (FIG. 4, lanes 1-5, respectively) (w/w) ratios. The ability of these FGF2-K complexes to bind DNA was determined by measuring the ability of FGF to promote the uptake of plasmid DNA into cells. FGF2-K conjugates were evaluated at various protein to DNA ratios for their ability to deliver pSVβ-gal DNA into cells (FIG. 4).

Briefly, the complexes were incubated for 1 hr at room temperature and then added to COS cells for 48 hrs. Cell extracts were prepared and assayed for β-gal enzyme activity. Briefly, cells are washed with 1 ml of PBS (Ca⁺² and Mg⁺² free) and lysed. The lysate was vortexed and cell debris removed by centrifugation. The lysate was assayed for β-gal activity as recommended by the manufacturer (Promega, Madison, Wis.). The β-gal activity was normalized to total protein. As seen in FIG. 4, lane 3, a 2:1 (w/w) ratio of FGF2-K:DNA gave maximal enzyme activity.

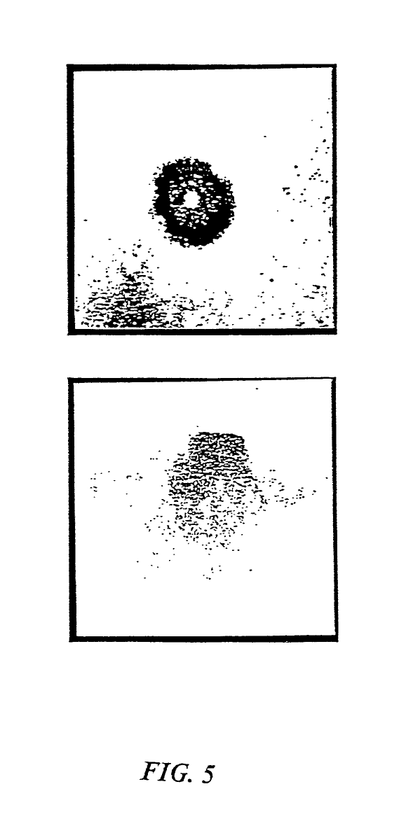

In addition, toroid formation, which correlates with increased gene expression, was assessed by electron microscopy. A representative toroid at a protein to DNA ratio of 2:1 is shown in FIG. 5, upper panel. Toroidal structures are absent, or only partially formed, at low ratios (e.g., 0.5:1) (FIG. 5, lower panel).

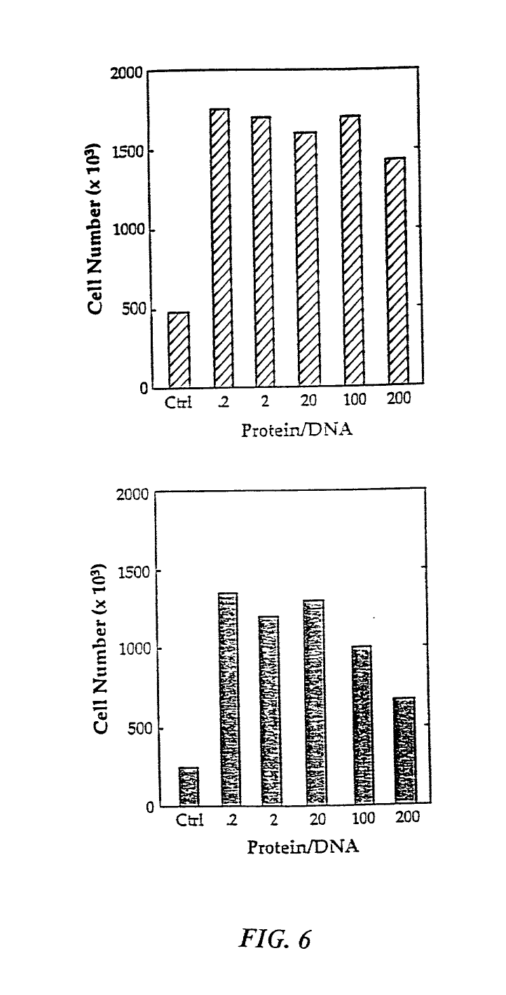

A proliferation assay is performed to determine if the condensed nucleic acid had an effect on the ability of FGF2-K to bind to cognate receptor and stimulate mitogenesis. The proliferation assay shows that only the highest dose of nucleic acid (200 μg) has a slightly inhibitory effect on proliferation as compared to FGF2-3 plus poly-L-lysine+DNA (FIG. 6).

A FGF2-K84-DNA at a protein:DNA ratio of 2:1 is introduced into COS cells and an endothelial cell line, ABAE, both of which express FGF receptors. The cells are subsequently assayed for β-galactosidase enzyme activity. COS and ABAE cells are grown on coverslips and incubated with the different ratios of FGF2-K:DNA for 48 hours. The cells are then fixed and stained with X-gal. Maximal β-galactosidase enzyme activity is seen when 50 μg of pSVβ per 100 μg of FGF2-3-polylysine conjugate is used.

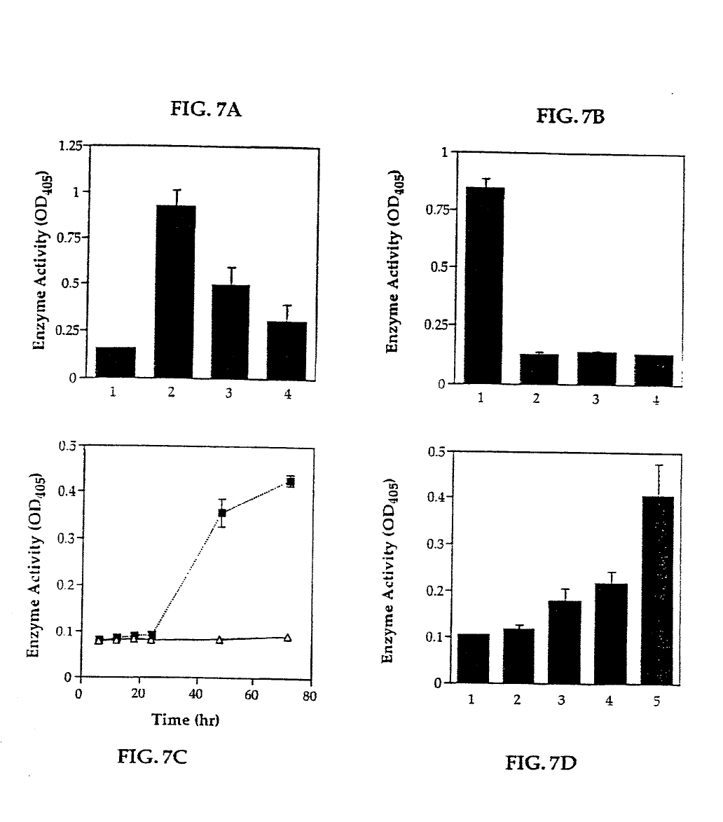

FGF2-K84-pSVβ-gal at a protein to DNA ratio of 2:1 was added to various cell lines and incubated for 48 hr. Cell extracts were prepared, assayed for β-gal activity and total protein. As shown in FIG. 7A, COS, B16, NIH3T3, and BHK cell lines were all able to take up complex and express P′-gal.

The expression of β-gal requires FGF2 for targeting into cells. pSVβ or pNASSβ plasmid DNA was incubated with (FIG. 7B, lanes 1, 2) or without (lanes 3, 4) FGF2-K84 for 1 hr at room temperature. Complexes were added to COS cells for 48 hr. Cell extracts were assayed for β-gal activity and normalized to total protein. Only background β-gal activity was seen unless the plasmid was complexed with FGF2/K84. Expression of β-gal is seen to be both time and dose-dependent (FIGS. 7C and 7D).

Sensitivity of the receptor mediated gene delivery system is determined using the optimized FGF2-K/DNA ratio for complex formation. Increasing amounts of the FGF2-K-DNA complex is added to cells. 100 μg of FGF2-K was mixed with 50 ug of pSVβ for 1 hour at room temperature. The COS and endothelial cells are incubated with increasing amounts of condensed material (0 ng, 1 ng, 10 ng, 100 ng, 1000 ng and 10,000 ng). The cells are incubated for 48 hours and then were assayed for β-galactosidase activity. In addition, cells grown on cover slips are treated with 1000 ng of FGF2-K-DNA for 48 hours, then fixed and stained using X-gal. The β-gal enzyme assay reveals that with increasing amounts of material there is an increase in enzyme activity. (FIG. 7D) Cells incubated with X-gal show blue staining throughout the cytoplasm in approximately 3% of the cells on the coverslip.

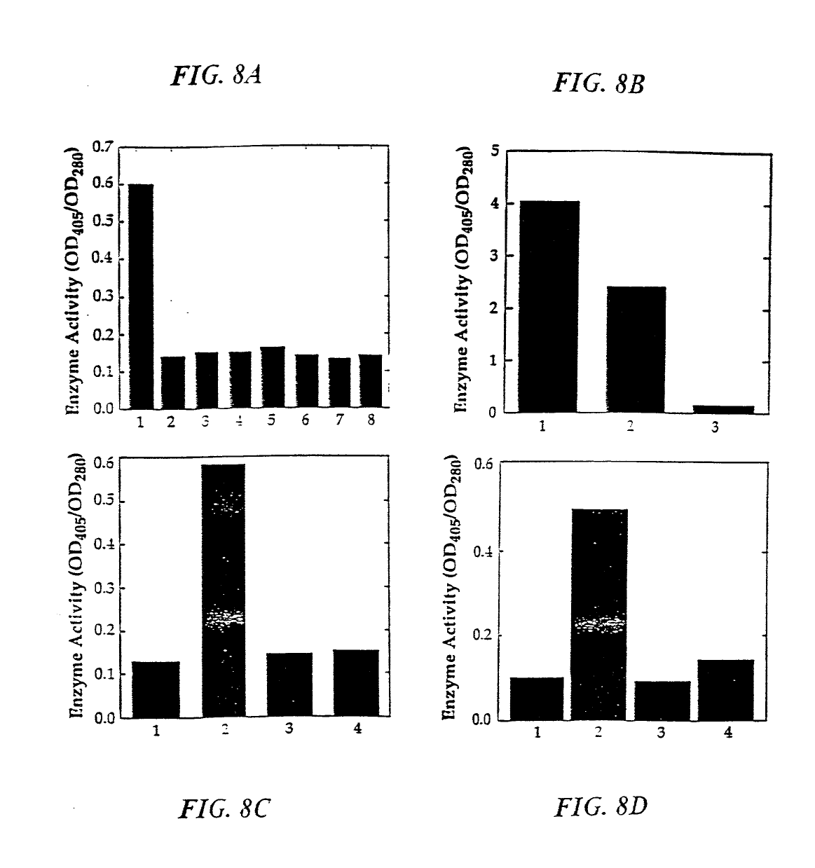

Targeting of the complexes is specific for the FGF receptor. First, as seen in FIG. 8A, FGF2-K84-pSVβ-gal resulted in enzyme activity (lane 1), while only background levels of activity were seen with FGF2+K84+DNA (lane 2), FGF2+DNA (lane 3), K84+DNA (lane 4), DNA (lane 5), FGF2-K84 (lane 6), FGF2 alone (lane 7) and K84 alone (lane 8). The expression of β-gal is specifically inhibited if free FGF2 is added during transfection (FIG. 8B). Moreover, the addition of heparin attenuates the expression of β-gal (FIG. 8C). Moreover, histone HI and cytochrome C were ineffective in delivering pSVβ-gal (FIG. 8C).

Taken together, these findings support the hypothesis that the targeted DNA is introduced into receptor-bearing cells via the high affinity FGF receptor. Because histone can bind heparin sulfate yet fails to elicit a signal, the introduction of DNA appears independent of the low affinity FGF receptor or non-specific endocytosis.

D. Effect of Endosome-disruptive Peptides

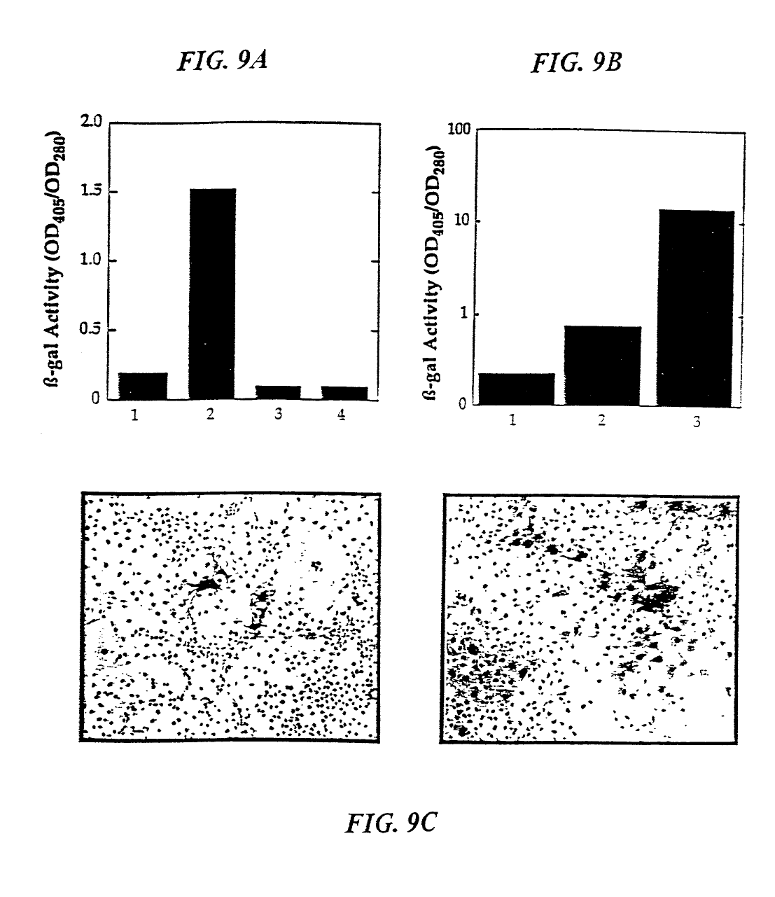

Targeting is mediated by passage of the complex through endosomes. Chloroquine, which was added to complexes before transfection, resulted in an 8-fold increase in β-gal activity (FIG. 9A).

Based on this, the effect of endosome disruptive peptides was evaluated. The peptide INF7, GLF EAIEGFIEN GWEGMIDGWYGC (SEQ ID NO. 32), derived from influenza virus, was synthesized. A complex between FGF2-K84 (5 μg) and pSVβ-gal plasmid DNA (5 μg) was formed. At this ratio, approximately half of the negative charge of the DNA was neutralized by the conjugate. K84, poly-L-lysine, was further added to saturate binding to the remaining DNA. The INF7 peptide was added 30 minutes later. The complex is added to COS cells and β-gal activity is assayed 48 or 72 hr later.

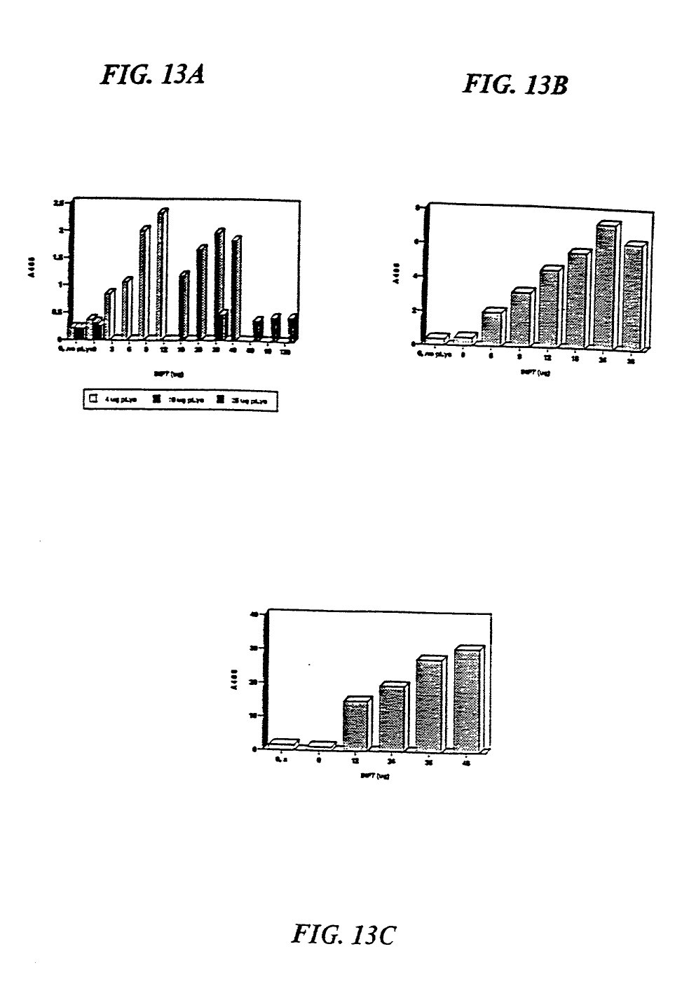

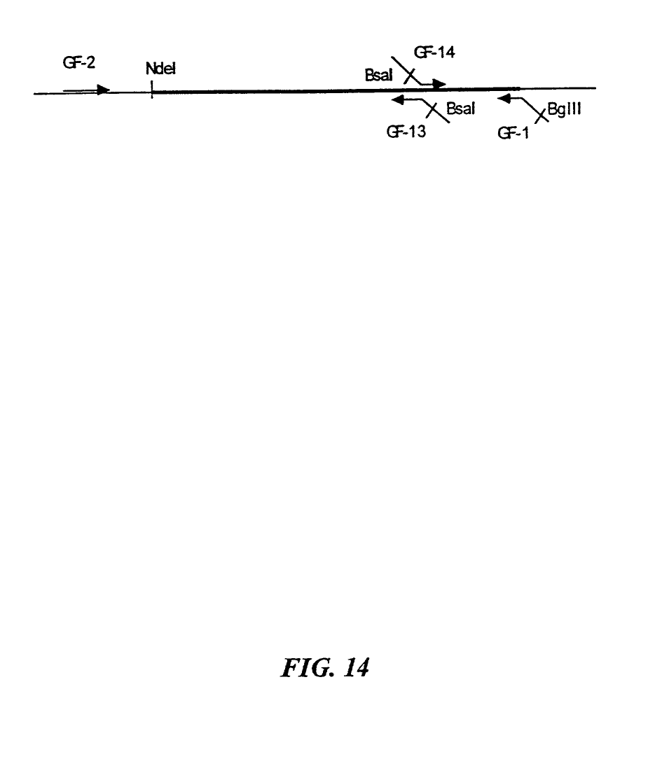

The amount of free polylysine necessary to neutralize the DNA and allow INF7 to complex was determined. Polylysine was added at 4, 10, or 25 μg to the FGF2-K84/pSVβ-gal complex. To each of these complexes four different concentrations of INF7 were added. Maximal β-gal expression was seen with 4 μg of K84 and 12 μg of INF7 (FIG. 13A). When higher amounts of poly-lysine were used, more cell death resulted. The optimal amount of INF7 was determined using 4 jig of polylysine. As seen in FIG. 13B, 24 μg of INF7 gave maximal β-gal activity. At 72 hr, 48 μg of INF7 gave maximal β-gal activity (approximately 20-32 fold enhancement) (FIG. 13C).

When an endosome disruptive peptide was included in the complex, expression of β-gal was increased 26-fold (FIG. 9B). Concomitant with this increased level of expression was an increase in the number of cells expressing β-gal. As seen in FIG. 9C, when endosome disruptive peptide (EDP) was present (right panel), 1%-5% of cells express β-gal in comparison to 0.1 %-0.3% without EDP added (left panel).

### Example 9

### Cytotoxic Activity of FGF/POLY-L-lysine Bound to SAP DNA Plasmid

The cytotoxicity assay measures viable cells after transfection with a cytocide-encoding agent. When FGF-2 is the receptor-binding internalized ligand, COS7 cells, which express FGFR, may be used as targets, and T47D, which does not express a receptor for FGF-2 at detectable levels, may be used as negative control cells.

Cells are plated at 38,000 cells/well and 48,000 cells/well in a 12-well tissue culture plate in RPMI 1640 supplemented with 5% FBS. The complex FGF2-K/pZ200M (a plasmid which expresses saporin) is incubated with COS7 or T47D cells for 48 hrs. Controls include FGF2-K alone, pZ200M alone, and FGF-2 plus poly-L-lysine plus pZ200M. Following incubation, cells are rinsed in PBS lacking Mg⁺⁺ and Ca⁺⁺. Trypsin at 0.1% is added for 10 min and cells are harvested and washed. Cell number from each well is determined by a Coulter particle counter (or equivalent method). A statistically significant decrease in cell number for cells incubated with FGF2-K/pZ200M compared to FGF2-K or pZ200M alone indicates sufficient cytotoxicity.

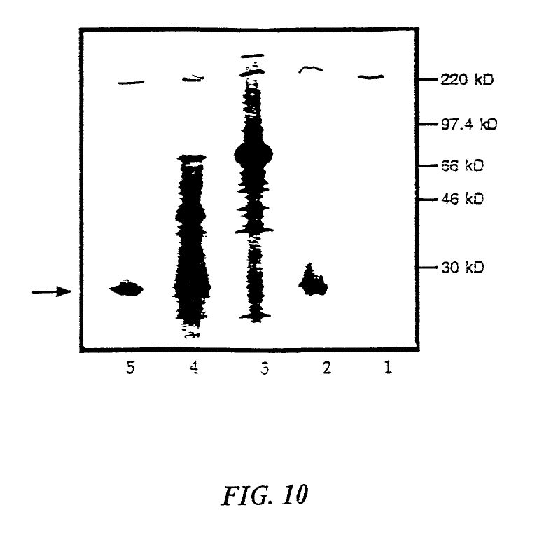

FGF2-polylysine-DNASAP complexes show selective cytotoxicity. To optimize the expression of the plant RIP, saporin, in mammalian cells, a synthetic saporin gene using preferred mammalian codons and introduced a “Kozak” sequence for translation initiation. The synthetic gene was then cloned into SV40 promoter and promoterless expression vectors. Because the expression of SAP from SAP-encoding DNA would only be feasible if the mammalian ribosome can synthesize the protein (SAP) prior to its inactivation by the SAP synthesized, the enzymatic activity of saporin encoded by the synthetic gene was tested. SAP was cloned into a T7/SP6 promoter plasmid and sense RNA was generated using T7 RNA polymerase. The RNA was then added to a mammalian in vitro translation assay. The results from this cell-free in vitro translation assay clearly show that the saporin expressed in a mammalian system can inhibit the expression of protein mutagenesis (FIG. 10). When added above to the lysate, SAP mRNA is translated into a protein that has the anticipated molecular weight of the saporin protein (lane 2). Similarly, when luciferase mRNA is added to the lysate, a molecule consistent with the luciferase protein is detected (lane 3). In contrast, if SAP mRNA is added to the lysate along with or 30 minutes prior to luciferase mRNA, saporin activity is detected (lanes 4 and 5).

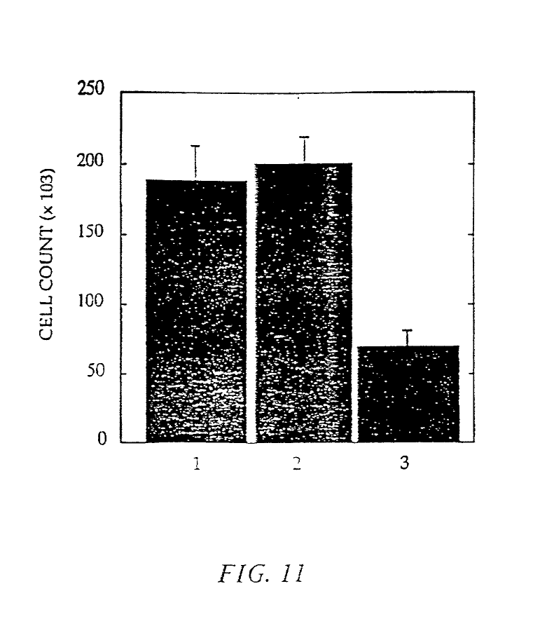

Transfection of cells with SAP DNA demonstrates cytotoxicity. When a mammalian expression vector encoding saporin is transiently expressed in NIH 3T3 cells using CaPO4, there is a >65% decrease in cell survival (lane 3) compared to cells mock transfected (lane 1) or transfected with DNA encoding β-gal (lane 2) (FIG. 11).

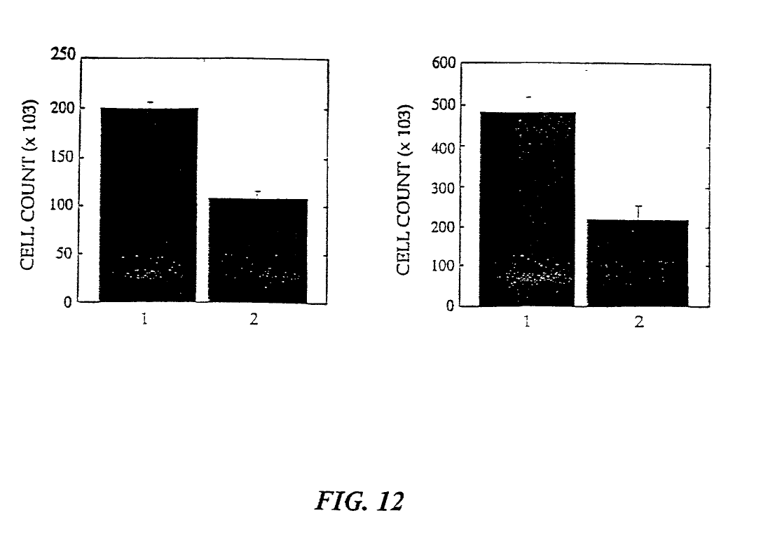

To determine whether the FGF2-K can transfer plasmid DNA encoding SAP into FGF receptor bearing cells, FGF2-K was condensed with the pSV40-SAP plasmid DNA at a ratio of 2:1 (w:w). BHK 21 and NIH 3T3 cells were used as the target cells. The cells (24,000 cells/well) were incubated with either FGF2-K-DNASAP or an FGF2-K-DNAβ-gal complex. After 72 hours of incubation, cell number was determined. As shown in FIG. 12, there is a significant decrease in cell number when cells are incubated with the FGF2-K-DNASAP complex compared to cells incubated with the FGF2-K-DNAβ-gal complex.

### Example 10

### Use of Tissue-specific Promoters in Mammalian Expression Vectors (pNASS-βand pNASS-SAP)

Two promoterless expression plasmids are used for insertion of tissue-specific promoters. These plasmids, pNASS-β (for β-galactosidase expression) and pNASS-SAP (for saporin expression), have unique EcoR I and Xho I sites for insertion of promoters. The plasmids containing a promoter are isolated from amplified cultures according to standard procedures and then purified for experimental use by double banding through CsTFA according to the manufacturer's protocol (Phannacia Biotech).

Smooth Muscle a-Actin

The sequence of the human smooth muscle a-actin promoter is known (Reddy et al., J. Biol. Chem. 265:1683-1687, 1990; GenBank accession number J05193). A luciferase expression plasmid containing α-actin promoter sequence from -894 bp to +12 bp (relative to the start of transcription) is obtained from Dr. C. Chandra Kumar (Schering-Plough Research Institute, Kenilworth, N.J.). This plasmid is used as a template for a PCR reaction designed to amplify the −670 bp to +12 bp fragment of the promoter. The primers incorporate an EcoR I site at the 5′ end and a Xho I site at the 3′ end. The sense primer used in this reaction was:

| | |
|---|---|
| 5′-TATATAGAATTCGTAGACAAAGCTAATGCACCAAAA-3′ | (SEQ ID |
| EcoRI | NO. 82) |

The antisense primer used was:

| | |
|---|---|
| 5′-TATATACTCGAGCACTGGGTGGTGTTCAGGGAAGCT-3′ | (SEQ ID |
| XhoI | NO. 83) |

A reaction mix is prepared in an 0.65 ml eppendorf tube and contains: 25 μl H₂O, 5 μl 10× PCR buffer with Mg⁺⁺ (Boehringer Mannheim), 10 μl 2 mM dNTP mix, 5 μl 20 μM stock of each primer. A wax bead is added, and the tube heated at 68° C. for 5 min. After cooling to room temperature, the remaining components are added: 34.5 μl H₂O, 5 μl 10× PCR buffer, 0.5 μl Taq DNA polymerase (Boehringer Mannheim), and 10 μl (200 ng) template DNA. The PCR reaction is carried out in an Ericomp TwinBlock EasyCycler using the following cycles: one cycle of a denaturation step (95° C. for 5 min), 20 cycles of a denaturation step, an annealing step, and an elongation step (95° C. for 1 min; 60° C. for 2 min; 72° C. for 2 min), and one cycle of an elongation step (72° C. for 7 min). An aliquot of the reaction is run on an agarose gel and the amplified product is purified using the Geneclean II Kit (Bio101). The purified DNA is then digested with EcoR I and Xho I and is subjected to another round of electrophoresis and Geneclean purification. The digested promoter fragment was ligated into EcoR I/Xho I digested pNASS-SAP and pNASS-β followed by transformation into competent DH5α cells and plating on LB plates containing ampicillin. Ampicillin-resistant colonies are screened for promoter insertion by PCR. Individual colonies are inoculated into 25 μl of a reaction mix consisting of 540 μl H₂O, 6.25 μl 10 mM dNTP mix, 62.5 μl 10× PCR buffer, 6.25 μl of each of the primers specified above, and 3.2 μl Taq DNA polymerase. Amplification conditions are: one cycle of a denaturation step (99° C. for 5 min), 20 cycles of a denaturation step, an annealing step, and an elongation step (99° C. for 1 min; 65° C. for 2 min; 72° C. for 2 min) and one cycle of an elongation step (72° C. for 7 min). Clones containing promoter insertions are identified by agarose gel electrophoresis of the PCR products and are used to inoculate cultures for DNA preparation. DNA is isolated from 50 ml cultures using Qiagen's Maxi columns and protocols. The sequence of the promoter inserts is confirmed by dideoxy sequencing with Sequenase version 2.0 (US Biochemicals).

The sequence of the PCR-generated α-actin promoter is:

| | |
|---|---|
| EcoRI | |
| GAATTCGTAGACAAAGCTAATGCACCAAAAAAATGAATGTAGTTATAGTAATGCTAACATCCAAATTCCT | (SEQ ID NO. 84) |
| CTTTGTAAGACATAGGCCTGTCAACCTTGTCTCCATACTTCAATTCCTATTTCCACTCACCTCCCTCAAG | |
| AACTTGATTTATAAACAGTGTGCCTACCATAAAATCATCACTCCCTCTATGTATTTATAGACGACTGAAG | |
| GAATATCTTTCTTCTTTGCATGCTACCGTGGTAGAAGGGTTTTAAAAGTCCGTGCTAGGCAGAGGCAGCC | |
| CTTTCTGCCCCTTTCTGTTCTCAGTTTATTAGGAAATGGCCTGAAATTCCAGCATGATAGCAAGCTGGCA | |
| TCCTCTGTGGAATGTGCAAACCATGCCTGCATCTGCCCATTACCCTAGCTCAGTGTCTCTGGGCATTTCT | |
| GCAGTTGTTCTGAAGGCTTGGCGTGTTTATCTCCCACAGGCGGCTGAACCGCCTCCCGTTTCATGAGCAG | |
| ACCAGTGGAATGCAGTGGAAGAGACCCAGGCCTCCGGCCACCCAGATTAGAGAGTTTTGTGCTGAGGTCC | |
| CTATATGGTTGTGTTAGACTGAACGACAGGCTCAAGTCTGTCTTTGCTCCTTGTTTGGGAAGCAAGTGGG | |
| AGGAGAGCAGGCCAAGGGGCTATATAACCCTTCAGCTTTCAGCTTCCCTGAACACCACCCAGTGCTCGAG | |
| XhoI | |

Tyrosinase Promoter

The sequence of the human tyrosinase promoter is known (Ponnazhagan et al., J. Investigative Dermatology, 102: 744-748, 1994; GenBank accession number U03039). A chloramphenicol acetyltransferase expression plasmid (pHTY-CAT) containing tyrosinase promoter sequence from an XmnI site at -2020 bp to a Pst I site at +13 bp is obtained from Dr. Byoung S. Kwon (Indiana University, Indianapolis, Ind.). The tyrosinase promoter is excised fom pHTY-CAT by digestion with Pst I, which cuts just upstream of the promoter as well as at position +13. This Pst I fragment is then cloned into the Pst I site of pBluescriptIl SK+(Stratagene). Following transformation into DH5α, DNA isolated from individual clones is screened for inserts by digestion with Sal I. There are two Sal I sites in the desired end product, one located near the 5′ end of the promoter fragment isolated from pHTY-CAT and the second located within the multicloning site of pBluescript. In the desired orientation, the Bluescript site is positioned downstream of the promoter such that the entire promoter can be excised by digestion with Sal I. The ends generated by Sal I are compatible with those generated by digestion with XhoI. Therefore, the Sal I promoter fragment is cloned into Xho I-digested pNASS-SAP and pNASS-β. Following transformation into DH5α, clones with the promoter in the desired orientation are identified by restriction analysis.

αA-Crystallin Promoter

The sequence of the human αA-crystallin promoter is known (Jaworski et al., J. Mol. Evolution, 33: 495-505, 1991; GenBank accession number S79457). The region corresponding to −400 bp to +50 bp is synthesized by PCR using overlapping oligos. For cloning purposes, an EcoR I site is introduced at the 5′ end and an Xho I site at the 3′ end of the promoter. The promoter is synthesized as two fragments (A and B) that overlap at a Pvu II site. The two fragments are then ligated together at the Pvu II site to give the intact promoter. Fragment A covers the 5′ half of the promoter and is synthesized using the following primers:

```
Sense A1:
                EcoRI
5′-TATAGAATTCCTGTGTCTAACGGGGGTGTGTGCTCTCCCTCCTCTGGCGACCATGAGGAAACCCCCG
GCAGGACAAGGTG-3′ (SEQ ID NO. 85)
Sense A2:
5′-CCTGCCCAGTGACTGGCAGATGAGAAGCTCCATTGTCGCCCCAGGGAGTATGGGGCACAGGCGCCTC
CTTGGGTTG-3′ (SEQ ID NO. 86)
Antisense A3:
5′-ATCTGCCAGTCACTGGGCAGGGGCTACGTGCCAGGGACCATGCTAGTTCTCTGCACACCTTGTCCTG
CCGGGGGTT-3′ (SEQ ID NO. 87)
Antisense A4:
                BamHI PvuII
5′-TATAGGATCCTGGACTCAGCTGAGGCCCGCCTGGGCACCCTGGGGCTCCCGGGAGGCAGACAACCCA
AGGAGGCGCCTGTG-3′ (SEQ ID NO. 88)
Fragment B covers the 3′ half of the promoter and is synthesized using the following
primers:
Sense B1:
                BamHI PvuII
5′-TATAGGATCCGGGCCTCAGCTGAGTCCAGGCCTCGGGGACAGTCCGTGCACG
CTCCTGGGGCTGGGGGCGGGC-3′ (SEQ ID NO. 89)
Sense B2:
5′-TTCATGAGCTCACGCCTTTCCAGAGAAATCCCTTAATGCCGCCATTCTGCTG
GTGGCATATATAGGGAGGGCTCGGCCTTG-3′ (SEQ ID NO. 90)
Antisense B3:
5′-GGAAAGGCGTGAGCTCATGAAGAAGGCTGCTCAGTCAGCAGAAACGTGGC
TGGGACAAGTGCCCGCCCCCAGCCCCAGGAG-3′ (SEQ ID NO. 91)
Antisense B4:
                  XhoI
5′-TATATACTCGAGCGGGGACCTGGAGGCTGGCAGGAGTCAGCGGGGCCTCT
GGCAGCCAGTGTGGAGCCAAGGCCGAGCCCTCCCTATA-3′ (SEQ ID NO. 92)
```

A reaction mix for amplification is prepared by combining 15 μl H₂O, 5 μl 10× PCR buffer with Mg⁺⁺, 10 μl 2 mM dNTP mix, 5 μl of 20 μM stock of each external primer (A1 and A4 or B1 and B4), and 5 μl of 0.2 μM stock of each internal primer (A2 and A3 or B2 and B3). A wax bead is added and the samples heated at 68° C. for 5 min and then cooled to room temperature. After cooling, the following is added to each reaction: 44.5 μl H₂O, 5 μl 10× PCR buffer, 0.5 μl Taq polymerase. The first PCR cycle is a denaturation step (95° C. for 5 min), followed by 25 cycles of a denaturation step, an annealing step, and an elongation step (95° C. for 1 min; 70° C. for 2 min; 72° C. for 2 min). An additional elongation step is performed for one cycle (72° C. for 7 min). The reactions are run on an agarose gel and the amplified product is purified using Geneclean II (Bio101). The purified DNAs are then digested with EcoR I and BamH I (fragment A) or Bam HI and Xho I (fragment B) and cloned into the corresponding sites of pBluescript II SK+ (Stratagene). The resulting ligations are transformed into competent DH5α cells and clones containing insert are identified by restriction digestion of DNA preparations. Each clone is further characterized by DNA sequence analysis to confirm identity. The crystallin promoter fragments are excised from the Bluescript intermediates using EcoRI and Pvu II for fragment A and Pvu II and Xho I for fragment B. These fragments are then ligated into the EcoRI and Xhol sites of pNASS-β or pNASS-SAP to give the intact crystallin promoter driving expression of the β-gal or saporin genes. The sequence of the inserted crystallin promoter is:

| | |
|---|---|
| EcoRI | |
| GAATTCCTGTGTCTAACGGGGGTGTGTGCTCTCCCTCCTCTGGCGACCATGAGGAAACCCCCGGCAGGAC | (SEQ ID NO. 93) |
| AAGGTGTGCAGAGAACTAGCATGGTCCCTGGCACGTAGCCCCTGCCCAGTGACTGGCAGATGAGAAGCTC | |
| CATTGTCGCCCCAGGGAGTATGGGGCACAGGCGCCTCCTTGGGTTGTCTGCCTCCCGGGAGCCCCAGGGT | |
| GCCCAGGCGGGCCTCAGCTGAGTCCAGGCCTCGGGGACAGTCCGTGCACGCTCCTGGGGCTGGGGGCGGG | |
| CACTTGTCCCAGCCACGTTTCTGCTGACTGAGCAGCCTTCTTCATGAGCTCACGCCTTTCCAGAGAAATC | |
| CCTTAATGCCGCCATTCTGCTGGTGGCATATATAGGGAGGGCTCGGCCTTGGCTCCACACTGGCTGCCAG | |
| AGGCCCCGCTGACTCCTGCCAGCCTCCAGGTCCCCGCTCGAG | |
| XhoI | |

c-myc promoter

The sequence of the human c-myc gene including the promoter region is known (Gazin et al., EMBO J., 3: 383-387, 1984; Genbank accession number X00364). A plasmid containing the human c-myc gene in pBluescriptll KS+ is obtained from Dr. Mark Groudine (Fred Hutchinson Cancer Research Center, Seattle, Wash.). The promoter region extending from −2.1 kb to +49 bp is isolated by digestion with Hind III and Nae I and subcloned into the HindIII/Nae I sites of pBluescript II SK+. The c-myc promoter is then excised from this intermediate as an EcoRl to Nae I fragment (the EcoRI site is upstream of the promoter within the Bluescript multicloning site) and ligated into EcoRI and blunt-ended Xho I sites of pNASS-β and pNASS-SAP.

### Example 11

### Construction of Non-mitogenic FGF2 Mutants

Five individual mutants of FGF2 are constructed to reduce or eliminate mitogenic capability. The five mutations are: (1) R116I (Heath et. al. Biochemistry, 1991); (2) R118K/K119E; (3) K119E (Springer et al., 1994); (4) Y120A (Springer et al., supra); (5) W123A (Springer et al., supra).

A “semi-inverse” PCR technique is used to change the coding sequence of FGF2 using four primers to create two fragments. Two of the primers, which prime on complementary strands incorporate the individual mutation, overlap only at the amino acid to be changed. In addition, these two primers include a non-complementary overhang containing a Bsa I restriction enzyme site. Bsa I recognizes the sequence 5′-GGTCTC-3′ and cuts downstream of its recognition sequence, thus removing the recognition sequence. The primers are designed such that digestion with Bsa I leaves complementary overhangs that, when ligated together, restore the original 155 amino acid FGF2, except containing one amino-acid change.

The two fragments are completed using two other primers, GF-1 and GF-2. The GF-2 primer anneals upstream of the ATGINdeI site in pFC80 and allows extension toward the mutation primer in FGF2 (primer GF-13 for R161I mutant). The GF-1 primer anneals at the 3′ end of FGF2 and incorporates a BgllI site. Thus, GF-i reverse-primes toward the second mutation primer (GF-14 for R161I mutant). Each fiagment is digested with Bsa I and with either Nde I (fragment generated by GF-2/GF-13 primers) or Bgl II (fragment generated by GF-1/GF-14 primers). The two fragments are ligated together into pET 11a digested with Nde I and Bam HI.

To create K119E, primer GF-15 replaced GF-14 and GF-16 replaced GF-13. To create Y120A, primer GF-17 replaced GF-14 and GF-18 replaced OF-13. To create W123A, primer GF-19 replaced GF-14 and GF-20 replaced GF-13. The double mutant R118K/K11 9E was created during PCR using GF-15 and GF-16 primers.

| | |
|---|---|
| Primer Sequences (All 5′ to 3′). | |
| GF-1: ATTAATTATAGATCTCAGCTCTTAGCAGACATTGG | (SEQ ID NO.94) |
| GF-2: GCTTGGGCATACATTCAATCAATTGTTATC | (SEQ ID NO.95) |
| R116I Primers (codon change of CGG to ATA): | |
| GF-13: CGTAATATGGTCTCAATATGTAAGTATTGTAGTTATTAGA | (SEQ ID NO.96) |
| GF-14: CGTAATATGGTCTCAATATCAAGGAAATACACCAGTTGG | (SEQ ID NO.97) |
| K119E Primers (codon change of AAA to GAA): | |
| GF-15: CGGATATGGTCTCAGAATACACCAGTTGGTATGTG | (SEQ ID NO.98) |
| GF-16: CGTAATATGGTCTCAATTCCCTTGACCGGTAAGTATTG | (SEQ ID NO.99) |
| Y120A Primers (codon change of TAC to GCA): | |
| GF-17: CGAATATGGTCTCAGCAACCAGTTGGTATGTGGCA | (SEQ ID NO.100) |
| GF-18: CGTAACATGGTCTCATTGCTTTCCTTGACCGGTAAGT | (SEQ ID NO.101) |
| W123A Primers (codon change of TGG to GCA): | |
| GF-19 GCTATTAGGTCTCAGCATATGTGGCATTGAAACGAAC | (SEQ ID NO.102) |
| GF-20 CGAATTAGGTCTCAATGCACTGGTGTATTCCTTGACC | (SEQ ID NO.103) |
| PCR Conditions | |
| 1 Cycle of: 95° C./5′ | |
| 5 Cycles of: 94° C./45″, 42° C./30″, 72° C./30″ | |
| 5 Cycles of: 94° C./30″, 45° C./1″, 72° C./15″ | |
| 10 Cycles of: 94° C./30″, 55° C./2′, 72° C./2′ | |
| 15 Cycles of: 94° C./45″, 60° C./1′, 72° C./30″ | |
| 1 Cycle of: 72° C./7′. | |

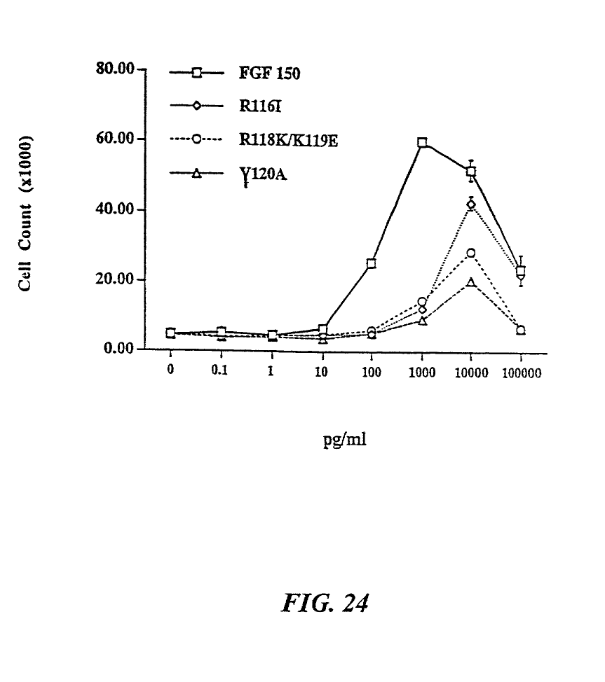

Three of the mutants and wild-type FGF2 are expressed and purified over a heparin column. Purified proteins are analyzed by Coomassie staining of an SDS-PAGE gel and by Western blot analysis (FIG. 24). Purified proteins are dialyzed to reduce the salt concentration and assayed for their ability to stimulate proliferation of ABAE cells. As shown in FIG. 25, mutants exhibit reduced ability to stimulate proliferation of endothelial cells compared to wild-type FGF2.

### Example 12

### Toxigene Expression in Angioplasty Model of Rabbit Common Iliac Arteries

On day 0, balloon catheter denudation is performed on both common iliac arteries in 12 New Zealand White rabbits. The appropriate vessel is accessed via the femoral artery. All animals are systemically dosed on day 3, day 4 and day 5 and sacrificed on day 7 by euthanasia with an overdose of pentobarbital (60 mg/kg). The iliac arteries are excised and recombinant β-galactosidase expression evaluated both by histochemical staining (X-gal and immunoreactivity with anti-β-gal antibody) and by enzymatic activity of β-galactosidase in tissue extracts.

Rabbits are anesthetized by intramuscular administration of 35 mg/kg Ketamine and 5 mg/kg Xylazine. Both femoral areas are shaved free of hair. The surgical site is scrubbed with chlorhexidine soap and swabbed with Betadine solution and 70% EtOH. Under sterile conditions, a longitudinal incision about 2-3 cm is made in the right groin region to provide access to the femoral artery. Additionally, systemic heparin (150 IU/kg) is provided via an ear vein.

A Fogarty 4F balloon catheter is introduced into the iliac artery through the superficial femoral artery to 6 cm beyond the arteriotomy. The balloon is inflated with approximately 0.35 ml of saline to provide moderate resistance at 0.35 ATM for 10 sec, then withdrawn 4.0-4.2 cm, and deflated. Inflation and deflation is repeated twice at 1 min. intervals. Following denudation, the superficial femoral artery is ligated with 4-0 silk suture at the site of catheter entry. The skin surrounding the incision is apposed with wound clips (Autoclip 18 mm).

On days 3, 4, and day 5 after balloon denudation, FGF2-K SV40-β-gal (β-gal gene under control of SV40 promoter), FGF2-K actin-β-gal (β-gal gene under control of actin promoter), and FGF2-K pNASS-β-gal (vector lacking a promoter) are administered at a dose of 50 μg/kg in a 1 ml volume. On day 7, the animals are intravenously injected with 60 mg/kg pentobarbital. The right and left iliac arteries in each rabbit are removed, and the proximal ends of each segment are marked with a suture.

For experiments involving histological examination of vessels, perfusion fixation is carried out in situ. In this procedure, arteries are cleared of blood via perfusion with normal saline followed by fixation in situ by perfusion with 2% formaldehyde, 0.2% paraldehyde in PBS, pH 7.4. All perfusion-fixed arteries are excised and immersed in fixative for a firther 2 hours. Arteries that are not fixed by perfusion prior to removal are immediately frozen for later measurement of β-galactosidase activity in tissue extracts.

β-galactosidase activity is measured in the following assay. Briefly, tissue is dispersed by homogenization for 1 min in 1 ml of 0.1 M phosphate buffer pH7.4, 0.2% Triton X-100. Tissue is further extracted at room temperature for 30 minutes and non-solubilized material is collected by centrifugation (14000 rpm×30 min. at 4° C.). β-galactosidase activity in the supernatant is determined using a chemiluminescent β-galactosidase assay kit (Clontech, Palo Alto, Calif.).

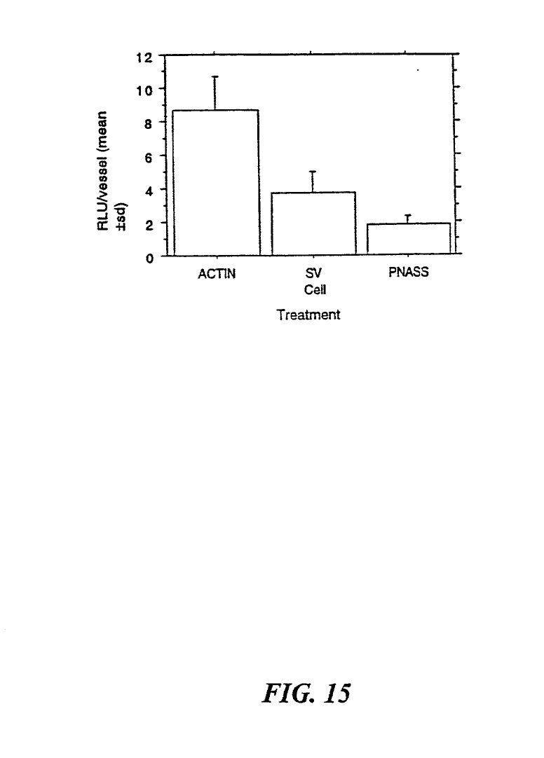

FIG. 15 shows the amount of Relative Light Units (RLU) per vessel. Animals treated with FGF2-K84-Actin β-gal show the highest level of β-gal activity. Some β-gal activity is also detected in animals adminstered with the promoterless construct (PNASS). Two possible explanations are: (1) an active cryptic promoter within the plasmid causing read through transcription of the β-gal gene, and (2) endogenous β-gal gene expression. However, by statistical analysis, using both the Fisher and student t-test, there is statistically significant more expression when an actin promoter is used over no promoter.

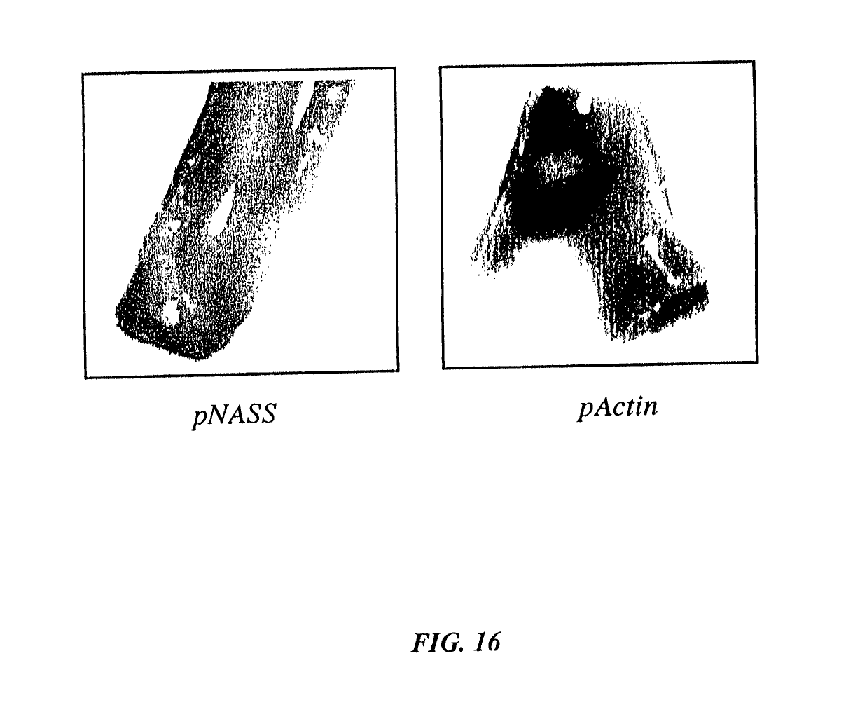

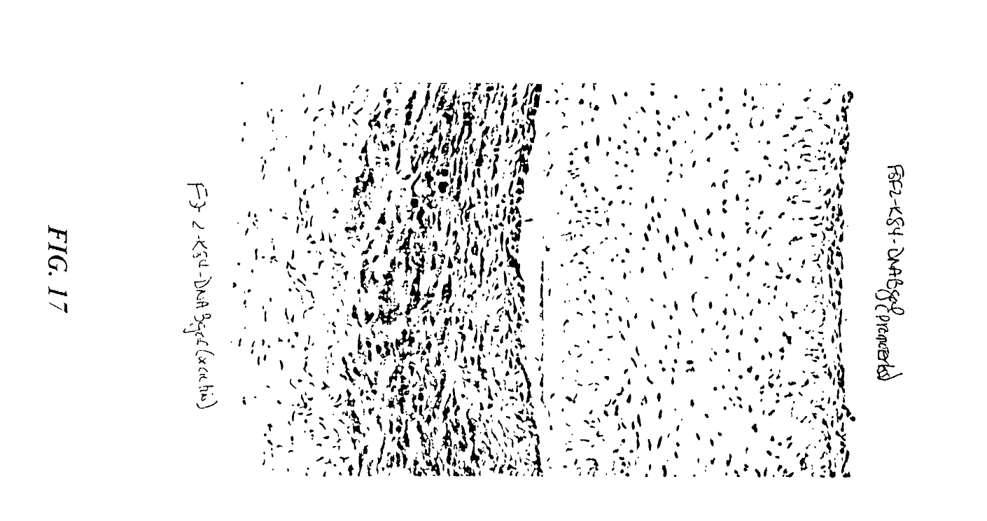

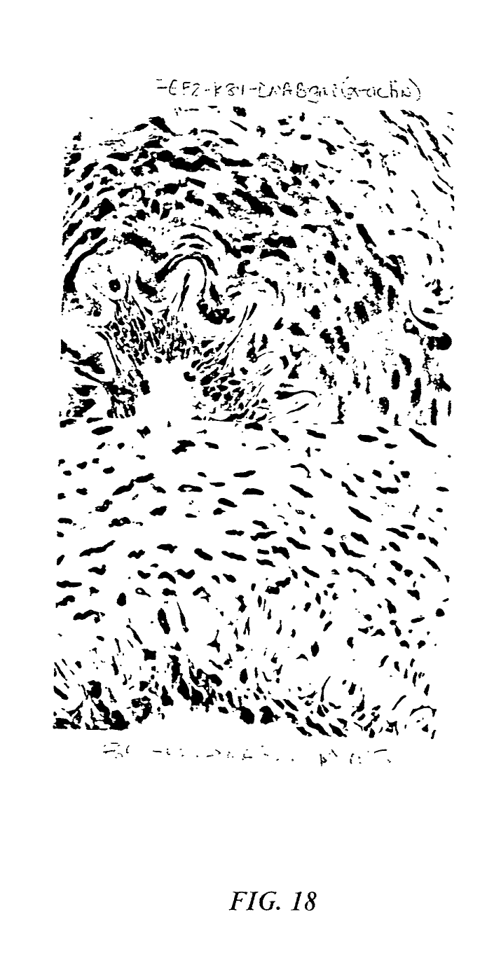

Tissues are also harvested and prepared for histochemical analysis. Prior to sectioning, arterial segments are stained with X-gal. As shown in FIG. 16, animals treated with FGF2-K84-Actin β-gal showed positive staining compared to lack of staining in animals treated with the promoterless constructs. Arterial sections are taken from animals treated with FGF2-K84-Actin β-gal and FGF2-K84-promoterless β-gal, stained with X-gal and counter stained with Fast Red. As shown in FIG. 17, X-gal staining is only apparent in the tissue from animals treated with the FGF2-K84-Actin β-gal complexes. To verify expression of β-gal, tissue sections are incubated with an antibody specific for the bacterial β-gal protein. As seen in FIG. 18, strong reactivity is detected in tissue sections from animals treated with FGF2-K84-Actin β-gal complexes. No immunoreactivity is seen in the vessels when FGF2-K84-pNASS β-gal is administered.

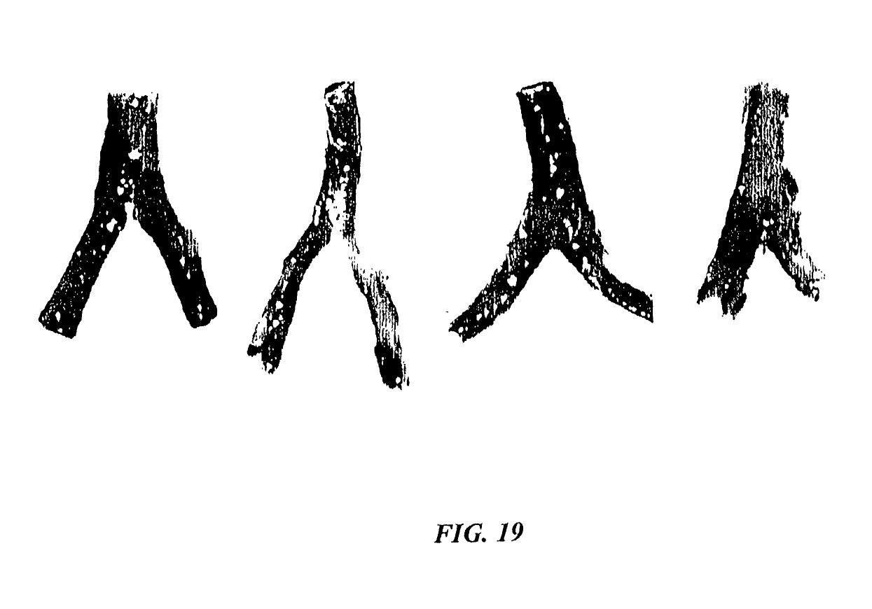

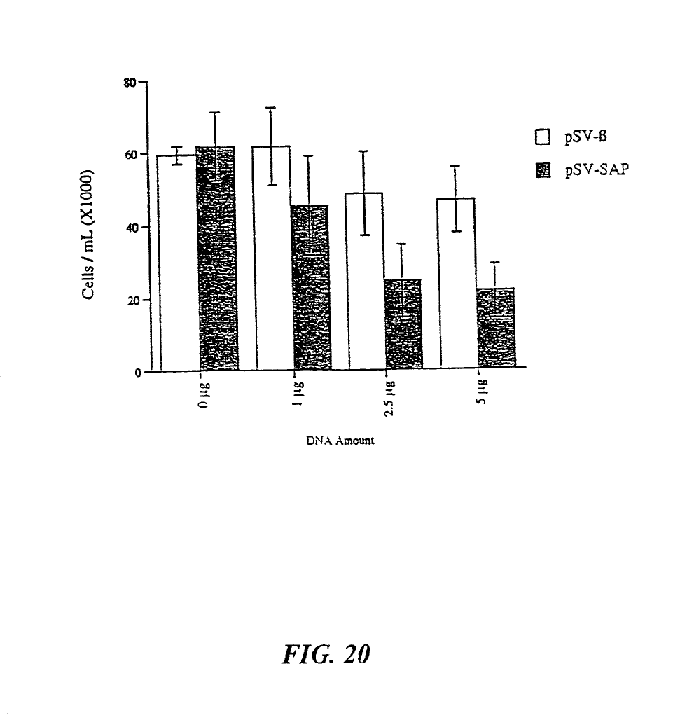

A second experiment is performed in which only one iliac artery is denuded. Either FGF2-K84-Actin β-gal or a mixture of FGF2-K84, Actin β-gal, and poly lysine is administered as above. As shown in FIG. 19, significant β-gal expression from the complex as compared to the mixture is seen in the injured artery.

### Example 13

### Toxigene Expression After Administration in Lung Metastases

Groups of BDF1 female mice (ten animals per test group, total 30 animals) were injected in the tail vein with 5×10⁵ B₁₆F₁₀ cells in 0.2 ml. On day 9, animals are weighed and randomized into treatment groups. On days 10, 11, and 12, animals are treated with 0.25 μg/kg of FGF2-K-SV40β-gal, or FGF2-K-pNassβ-gal. On day 14, animals are weighed and evaluated for toxicity.

β-galactosidase activity is measured either in lung cell extracts or by histochemistry stain as described above. The mean animal body weight is computed for days 9 and 14. The fraction of tumor volume in treated to control animals (T/C) is calculated for all test group with >65% survivors on day 14.

### Example 14

### In Vitro Delivery of SAP DNA

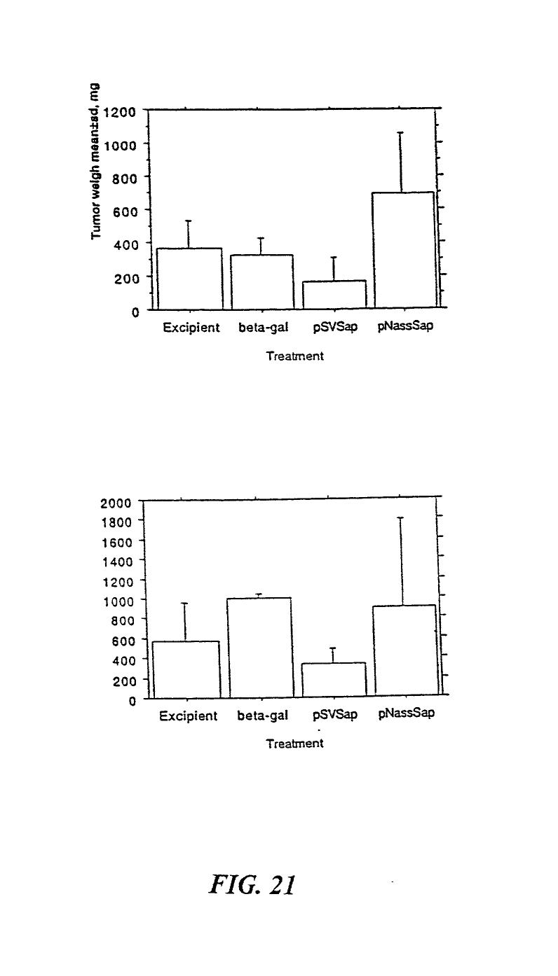

Biological activity of SAP in pSV-SAP DNA is tested by transient transfection in NIH3T3 cells. As a negative control, cells are also treated with FGF2-K₈₄-pNASS-SAP. As seen in FIG. 21, there are significantly fewer cells in the pSV-SAP transfected wells compared to the pSV-β wells. A duplicate plate of cells transfected with pSV-β was worked up for a β-gal assay which showed very high expression levels, thus confirming successful transfection.

### Example 15

### In Vivo Delivery of Toxigenes Complexed with SAP DNA Reduction in Tumor Size with Intratumoral Injection of FGF2-K-DNA SAP Complexes

Groups of three BDF1 mice are injected subcutaneously in the left fland with 5×10⁵ B₁₆F₁₀ cells suspended in PBS w/o Ca⁺⁺ and Mg⁺⁺ at a concentration of 25×10⁶/ml. When tumor diameters reach 0.5-1.0 nun, animals are randomized and given intratumoral injections of test compounds. The animals are dosed with a single injection of FGF2-K-SV40β-gal, FGF2-K-pNASS-SAP, FGF2-K-pSV40SAP (FGF2-K 15 μg-DNA 7.5 μg /50 μl) or excipient.

Groups:

| | |
|---|---|
| 1. Excipient | 50 μl/tumor/mouse |
| 2. FGF2-K-SV40β-gal | 50 μl/tumor/mouse |
| 3. FGF2-K-NASSβ-gal | 50 μl/tumor/mouse |
| 4. FGF2-K-SV40SAP | 50 μl/tumor/mouse |

Within each group, animals receive treatment intra-tumor at time 0, and tumor is removed at 48 or 72 hours. Mice are checked daily; tumor size is measured in 2 directions by caliper once a day. The tumors are removed at 48 or 72 hours for histological examinations and divided into 2 segments. One segment is frozen and the other fixed in 10% NBF.

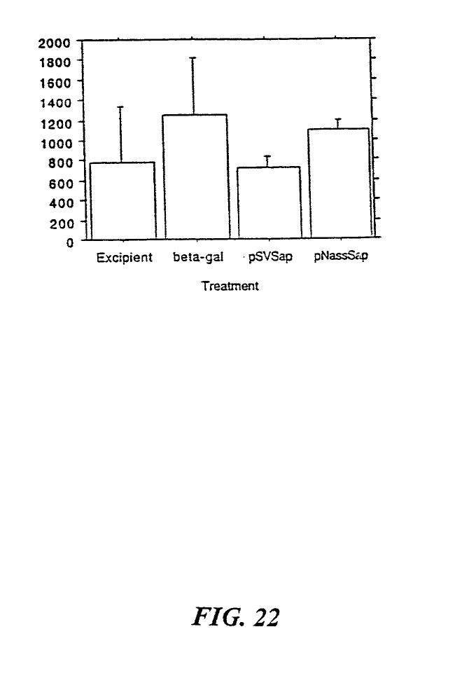

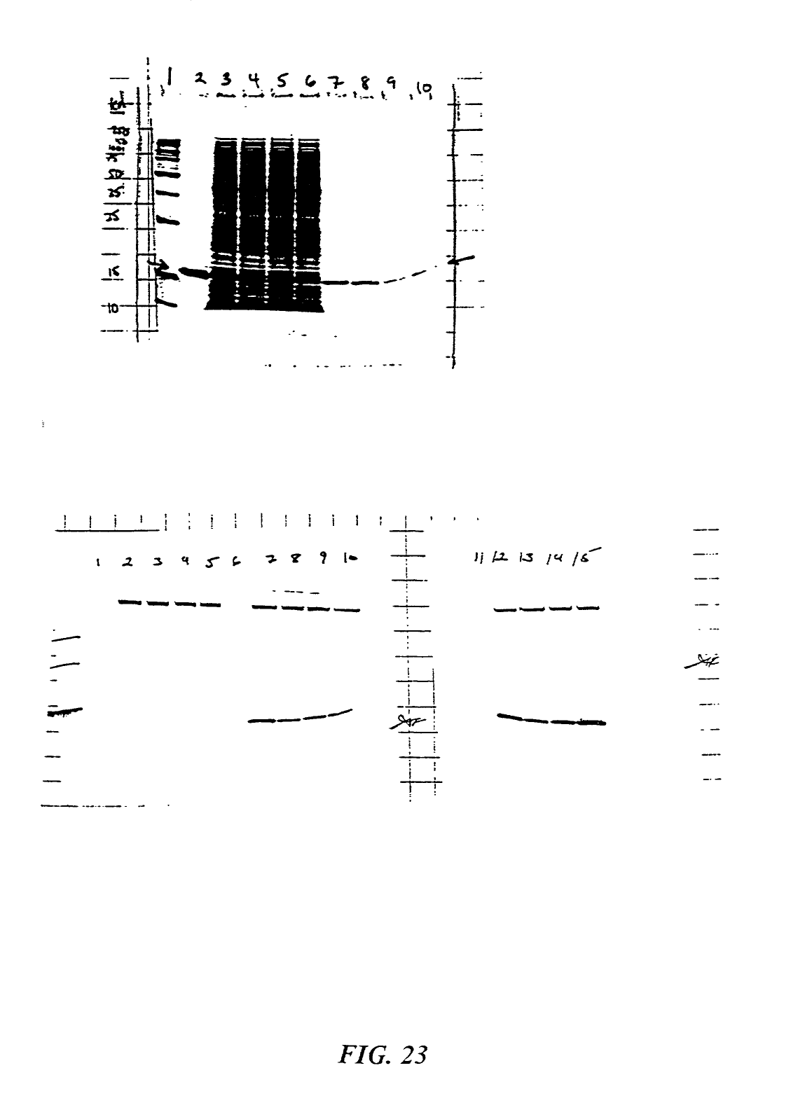

At the start of treatment all animals have tumors of approximately equivalent size. However, as shown in FIG. 22, at both 48 hr and 72 hr, animals receiving FGF2-K-SV40SAP have smaller tumors, as measured by weight and volume, than those animals receiving FGF2-K-SV40β-gal. Tumor size is also decreased in the FGF2-K-SV40SAP treated group compared to the the FGF2-K-SV40β-gal treated group when the animals are given 2 injections of the material (FIG. 23).

### Example 16

### Condensation of Nucleic Acid

Ligand-polvcation condensation of nucleic acids is described. In this example FGF2-polylysine is used to condense DNA. Briefly, DNA is diluted in 0.1 M HEPES, 0.1 M NaCl. Depending on the final volume, either a 15 ml conical tube or a 1.5 ml eppendorf centrifuge tube is used. FGF2-polylysine conjugate is added to the tube. Diluted DNA is added dropwise into the tube containing the conjugate. During addition, the solution is mixed by gentle vortexing (no higher than a setting of 3). Once formed, 50 μl of the complex is added to cells in a 12 well tissue culture plate.

A typical condensation is prepared for addition to 4 wells. For this, 0.492 μg/ml of FGF2-K84 and 3 μg/ml of DNA is required. A total of 5 μg of FGF2-K is added per well at a protein to DNA ratio of 2:1.

### Example 17

### Production of Antibodies to Cell Surface Receptors

In this example, the generation of a monoclonal antibody specific for the FGF receptor is described. Furthermore, the antibody is internalized following binding to the cell surface.

Hybridomas are generated after injection of mice with ECDR1, the extracellular domain of FGFRI produced by expression in a baculorivus-insect expression system (Kiefer et al., Growth Factors 5:115, 1991) or with SK-HEP-1 cells, a human cell line derived from a liver adenocarcinoma (ATCC No. HTB52). Female BALB/c mice are injected subcutaneously with either 80 μg of ECDR1 in complete Freund's adjuvant or injected bp with 10⁷ SK-HEP-1 cells in 0.2 ml Dulbecco's PBS. For ECDR1, animals are injected four additional times at 11-14 day intervals, with the final injection without Freund's adjuvant. For SK-HEP-1 cells, animals are injected twice more 14 and 28 days later. Four days following the final immunization, the spleen is harvested, and a cell suspension made. Cells are fused with NS—O fusion partner using PEG-1500 (see, Harlow and Lane, Antibodies: A Laboratory Manual, CSH Press, 1987, for protocols). Hybridoma cells are selected in RPMI-1640 containing HAT and 0.005% 2-ME followed by selection in RPMI-1640 containing HT.

An enzyme linked immunosorbent assay (ELISA) is used for screening the hybridomas. Briefly, plates are coated with 50 μl of ECDR1 (200 ng/ml) overnight at 4° C. After washing, conditioned media samples are added. A second antibody conjugated to horseradish peroxidase (Bio-Rad, 1:1000 dilution) is used to select hybridomas. Cells in positive wells are cloned by limiting dilution. Antibodies are purified by ammonium sulfate precipitation and Affi-Gel Protein A agarose column (Bio Rad, Richmond, Calif.) chromatography according to the manufacturer's protocol. The purity of the antibody is assessed by electrophoresing the preparation on a 7.5% PhastGel (Pharmacia, Uppsala, Sweden) under non-reducing conditions and staining with Coomassie blue.

A single specific hybridoma is generated from mice injected with ECDR1. Although this antibody bound the extracellular domain and FGFR by Western blot, the antibody did not bind to FGFR on the surface of whole cells.

A large number of hybridomas are generated from mice immunized with SK-HEP-1. An immunotoxin strategy is employed to identify hybridomas that are internalized. This technique relies on the ability of a conjugate between anti-mouse IgG and saporin (“second immunotoxin”), when added with hybridoma conditioned medium, to fill target cells. Primary antibody present in the conditioned media recognizes FGFR and binds to the cell surface; second immunotoxin will bind to the primary antibody and is internalized by “piggy-backing” on the first. Upon internalization, saporin inhibits protein synthesis, mediating cell death. As a secondary screen, this approach selects for hybridomas that secrete IgGs able to recognize cell surface high affinity FGF receptor and be internalized.

Immunotoxin is prepared by conjugating saporin with anti-mouse IgG antibody. Briefly, saporin is isolate and purified from the seeds of the plant Saponaria officinalis. A five- to six-fold molar excess of N-succinimidyl-3(pyridyldithio)propionate (SPDP) (Pharmacid, Piscataway, N.J.) is used to derivative the antibodies according to the manufacturer's instructions. The range of derivatization for saporin is 0.9-1.1 moles of pyridyl dithione/mole of protein, for the antibodies ranges from 2 to 4. After reducing saporin-SPDP with 0.1 M dithiothreitol, the mixture is passed over Sephadex G25 (Pharmacia) equilibrated in 10 mM acetic acid, 0.14 M sodium chloride, pH 5.0. A sufficient amount of thiolated saporin is pooled and added to the SPDP-modified antibody so that the molar excess of thiolated saporin to derivatized antibody is 3:1. The mixture is vortexed and incubated on a platform agitator for two hours at room temperature and left overnight at 4° C.

The immunotoxin is then purified by MonoS chromatography (Pharmacia) as described herein. The 11A8-SAP immunotoxin is purified by affinity chromatography on a Protein G affinity column (Pharmacia). Fractions that contained only antibody-conjugated saporin and free antibody were pooled, dialyzed against borate buffered saline, and loaded onto an AffiGel 10 (Bio-Rad) anti-saporin column prepared according to the manufacturer's instructions with rabbit polyclonal anti-saporin). The column is thoroughly washed, and protein eluted with 1.0 M acetic acid.

Cell lines that express FGF high affinity receptors were treated with 11A8-saporin, with saporin alone, or with antibody alone and cytotoxicity was measured by inhibition of thymidine incorporation or an MTT or MTS assay.

One hybridoma, 11A8, proved to be the most cytotoxic by this assay. 11A8 recognizes ECDR1 by Western blot, can immunoprecipitate FGFRs from extracts of SK-HEP-1 and SK-MEL-28, a melanoma cell. Furthermore, 11A8 also stains SK-HEP-1 cells on the cell surface.

When 11A8 is conjugated to saporin, it is a potent cytotoxic agent for SK-MEL-28 and SK-HEP-1 cells. The ED₅₀ is approximately 30 pM as assayed by inhibition of thymidine incorporation. As well, 11A8-saporin conjugate reduces tumor volume in nu/nu mice injected with SK-MEL-28 cells and treated either 7 and 11 days later (groups A, B, C, and E) or 7, 11, 15, and 19 days later (groups D and F). As shown in the table below, 11 A8-SAP is cytotoxic in vivo evidencing internalization.

| | | | Mean Tumor | |
|---|---|---|---|---|
| Group | Sample | Dose | Volume* | Significance |
| A | Dulbecco's | | 48.1 ± 13.4 | |
| | PBS | | | |
| B | Saporin | 2 × 65.5 μg/kg | 52.1 ± 9.24 | |
| C | rFGF-SAP | 2 × 105 μg/kg | 20.7 ± 9.37 | P < 0.005 |
| D | rFGF-SAP | 4 × 26.25 μg/kg | 11.0 ± 6.03 | P < 0.01 |
| E | 11A8-SAP | 2 × 448 μg/kg | 1.17 ± 1.03 | P < 0.01 |
| F | 11A8-SAP | 4 × 112 μg/kg | 4.25 ± 2.75 | P < 0.01 |

*errors are SEM

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method of treating tumors in a patient, comprising administering to the patient a pharmaceutical composition having the formula:
receptor-binding internalized ligand—nucleic acid binding domain—cytocide-encoding agent, wherein:
the receptor-binding internalized ligand is a polypeptide reactive with a cell surface receptor;
the nucleic acid binding domain binds to a nucleic acid, the domain being chemically conjugated or fused to the receptor-binding internalized ligand;
the cytocide-encoding agent is a nucleic acid molecule encoding a cytocide, the agent being bound to the nucleic acid binding domain; and wherein
the receptor-binding internalized ligand—nucleic acid binding domain—cytocide-encoding agent binds to the cell surface receptor and internalizes the cytocide-encoding agent in cells bearing the receptor.

2. A method of treating tumors in a patient, comprising administering to the patient a pharmaceutical composition having the formula:
receptor-binding internalized ligand—nucleic acid binding domain—prodrug-encoding agent, wherein:
the receptor-binding internalized ligand is a polypeptide reactive with a cell surface receptor;
the nucleic acid binding domain binds to a nucleic acid, the domain being chemically conjugated or fused to the receptor-binding internalized ligand;
the prodrug-encoding agent is a nucleic acid molecule encoding a prodrug, the agent being bound to the nucleic acid binding domain; and wherein
the receptor-binding internalized ligand—nucleic acid binding domain—prodrug-encoding agent binds to the cell surface receptor and internalizes the prodrug-encoding agent in cells bearing the receptor.

3. A method of treating tumors in a patient, comprising administering to the patient a pharmaceutical composition having the formula:
receptor-binding internalized ligand—nucleic acid binding domain—cytokine-encoding agent, wherein:
the receptor-binding internalized ligand is a polypeptide reactive with a cell surface receptor;
the nucleic acid binding domain binds to a nucleic acid, the domain being chemically conjugated or fused to the receptor-binding internalized ligand;
the cytokine-encoding agent is a nucleic acid molecule encoding a cytokine, the agent being bound to the nucleic acid binding domain; and wherein
the receptor-binding internalized ligand—nucleic acid binding domain—cytokine-encoding agent binds to the cell surface receptor and internalizes the cytokine-encoding agent in cells bearing the receptor.

4. The method of any one of claims 1, 2, or 3, wherein the receptor-binding internalized ligand is a polypeptide reactive with an FGF receptor.

5. The method of claim 1 wherein the cytocide-encoding agent encodes a protein that inhibits protein synthesis.

6. The method of claim 5 wherein the protein is a ribosome inactivating protein.

7. The method of claim 6 wherein the ribosome inactivating protein is saporin.

8. The method of claim 6 wherein the ribosome inactivating protein is gelonin.

9. The method of claim 6 wherein the ribosome inactivating protein is Pseudomonas exotoxin.

10. The method of claim 5 wherein the protein inhibits elongation factor 2.

11. The method of claim 10 wherein the protein is diphtheria toxin.

12. The method of claim 2 wherein the prodrug-encoding agent encodes HSV-thymidine kinase or cytosine deaminase.

13. The method of claim 3, wherein the cytokine-encoding agent encodes a cytokine selected from the group consisting of IL-2, IL-10, IL-12 and IFN-γ.

14. The method of claim 3, wherein the cytokine-encoding agent encodes B7 and a cytokine selected from the group consisting of IL-2, IL-10, IL-12 and IFN-γ.

15. The method of any one of claims 1, 2, or 3 wherein the receptor-binding internalized ligand is a polypeptide reactive with the FGF receptor and the nucleic acid binding domain is poly-L-lysine or protamine.

16. The method of any one of claims 1, 2, or 3 wherein the nucleic acid binding domain is selected from the group consisting of helix-turn-helix motif proteins, homeodomain proteins, zinc finger motif proteins, steroid receptor proteins, leucine zipper motif proteins, helix-loop-helix motif proteins, and β-sheet motif proteins.

17. The method of any one of claims 1, 2, or 3 wherein the nucleic acid binding domain is selected from the group consisting of AP-1, Sp-1, rev, GCN4, λcro, λcI, TFIIA, myoD, retinoic acid receptor, glucocosteroid receptor, SV40 large T antigen, and GAL4.

18. The method of any one of claims 1, 2, or 3 wherein the nucleic acid binding domain is a polycation.

19. The method of claim 18 wherein the polycation is selected from the group consisting of poly-L-lysine, poly-D-lysine, protamine, histone and spermine.

20. The method of claim 1 wherein the nucleic acid binding domain binds a DNA molecule that encodes a ribosome inactivating protein.

21. The method of claim 1 wherein the nucleic acid binding domain binds the coding region of saporin DNA.

22. The method of claim 1 wherein the cytocide-encoding agent further comprises a tumor-specific promoter.

23. The method of claim 2 wherein the prodrug-encoding agent further comprises a tumor-specific promoter.

24. The method of either of claims 22 or 23 wherein the tumor-specific promoter is selected from the group consisting of tyrosinase promoter, MAGE promoter, IL-2 receptor promoter, PSA-1 promoter, FGF receptor promoter, erbB2 promoter, erbB3 promoter, erbB4 promoter, MUC-1 promoter, HSP-27 promoter, CEA promoter, EGF receptor promoter, prostate specific antigen-1 promoter, probasin promoter, VEGF receptor promoter, int-1 promoter; int-2 promoter, IL-2 promoter, alpha-fetoprotein promoter, prostatic acid phosphatase promoter, prostate specific membrane antigen promoter, alpha-crystallin promoter and tie-2 promoter.

25. The method of any one of claims 1, 2, or 3, further comprising at least one linker that increases the serum stability, intracellular availability, or condensing ability of the nucleic acid binding domain, the addition of said linker(s) resulting in the formula:
receptor-binding internalized ligand—(L)_{q}—nucleic acid binding domain-cytocide encoding agent;
receptor-binding internalized ligand—(L)_{q}—nucleic acid binding domain-prodrug encoding agent, or the formula:
receptor-binding internalized ligand—(L)_{q}—nucleic acid binding domain-cytokine-encoding agent wherein:
L is at least one linker; and
q is 1 or more, such that the conjugate retains the ability to bind to a cell surface receptor and internalize the cytocide-encoding, prodrug-encoding or cytokine-encoding agent, and wherein the agent is bound to the nucleic acid binding domain.

26. The method of claim 25 wherein the linker increases the flexibility of the conjugate.

26. The method of claim 25 wherein the linker is selected from the group consisting of (GlyₘSerₚ)ₙ, (SerₘGlyₚ)ₙ and (AlaAlaProAla)ₙ in which n is 1 to 6, m is 1 to 6 and p is 1 to 4.

27. The method of claim 26 wherein m is 4, p is 1 and n is 2 to 4.


SEQUENCE LISTING (103 sequences)

SEQ ID NO: 1 - protein, 7 aa
  CDS 1..7: /product= nuclear translocation sequence
PKKRKVE

SEQ ID NO: 2 - protein, 8 aa
  CDS 1..8: /product= nuclear translocation sequence
PPKKAREV

SEQ ID NO: 3 - protein, 9 aa
  CDS 1..9: /product= nuclear translocation sequence
PAAKRVKLD

SEQ ID NO: 4 - protein, 5 aa
  CDS 1..5: /product= nuclear translocation sequence
KRPRP

SEQ ID NO: 5 - protein, 5 aa
  CDS 1..5: /product= nuclear translocation sequence
KIPIK

SEQ ID NO: 6 - protein, 7 aa
  CDS 1..9: /product= nuclear translocation sequence
GKRKRKS

SEQ ID NO: 7 - protein, 9 aa
  CDS 1..9: /product= nuclear translocation sequence
SKRVAKRKL

SEQ ID NO: 8 - protein, 9 aa
  CDS 1..9: /product= nuclear translocation sequence
SHWKQKRKF

SEQ ID NO: 9 - protein, 8 aa
  CDS 1..8: /product= nuclear translocation sequence
PLLKKIKQ

SEQ ID NO: 10 - protein, 7 aa
  CDS 1..7: /product= nuclear translocation sequence
PQPKKKP

SEQ ID NO: 11 - protein, 15 aa
  CDS 1..15: /product= nuclear translocation sequence
PGKRKKEMTKQKEVP

SEQ ID NO: 12 - protein, 12 aa
  CDS 1..12: /product= nuclear translocation sequence
GRKKRRQRRRAP

SEQ ID NO: 13 - protein, 7 aa
  CDS 1..7: /product= nuclear translocation sequence
NYKKPKL

SEQ ID NO: 14 - protein, 7 aa
  CDS 1..7: /product= nuclear translocation sequence
HFKDPKR

SEQ ID NO: 15 - protein, 7 aa
  CDS 1..7: /product= nuclear translocation sequence
APRRRKL

SEQ ID NO: 16 - protein, 6 aa
  CDS 1..6: /product= nuclear translocation sequence
IKRLRR

SEQ ID NO: 17 - protein, 6 aa
  CDS 1..6: /product= nuclear translocation sequence
IKRQRR

SEQ ID NO: 18 - protein, 5 aa
  CDS 1..5: /product= nuclear translocation sequence
IRVRR

SEQ ID NO: 19 - protein, 4 aa
  /note= “Cytoplasmic Translocation Signal”
KDEL

SEQ ID NO: 20 - protein, 4 aa
  /note= “Cytoplasmic Translocation Signal”
RDEL

SEQ ID NO: 21 - protein, 4 aa
  /note= “Cytoplasmic Translocation Signal”
KEEL

SEQ ID NO: 22 - DNA (cDNA), 804 bp; double-stranded, unknown
  CDS 1..804
  mat_peptide 46..804: /product= “Saporin”
gcatggatcctgcttcaattttcagcttggacaacaactgatgcggtcacatcaatcacattagatctagtaaatccgaccgcgggtcaatactcatcttttgtggataaaatccgaaacaatgtaaaggatccaaacctgaaatacggtggtaccgacatagccgtgataggcccaccttctaaagaaaaattccttagaattaatttccaaagttcccgaggaacggtctcacttggcctaaaacgcgataacttgtatgtggtcgcgtatcttgcaatggataacacgaatgttaatcgggcatattacttcaaatcagaaattacttccgccgagttaaccgcccttttcccagaggccacaactgcaaatcagaaagctttagaatacacagaagattatcagtcgatcgaaaagaatgcccagataacacagggagataaaagtagaaaagaactcgggttggggatcgacttacttttgacgttcatggaagcagtgaacaagaaggcacgtgtggttaaaaacgaagctaggtttctgcttatcgctattcaaatgacagctgaggtagcacgatttaggtacattcaaaacttggtaactaagaacttccccaacaagttcgactcggataacaaggtgattcaatttgaagtcagctggcgtaagatttctacggcaatatacggggatgccaaaaacggcgtgtttaataaagattatgatttcgggtttggaaaagtgaggcaggtgaaggacttgcaaatgggactccttatgtatttgggcaaaccaaag

SEQ ID NO: 23 - DNA, 765 bp; single-stranded, unknown
  CDS 1..762: /product= “Saporin”
atggtgacctccatcaccctggacctggtgaaccccaccgccggccagtactcctccttcgtggacaagatccgcaacaacgtgaaggaccccaacctgaagtacggcggcaccgacatcgccgtgatcggccccccctccaaggagaagttcctgcgcatcaacttccagtcctcccgcggcaccgtgtccctgggcctgaagcgcgacaacctgtacgtggtggcctacctggccatggacaacaccaacgtgaaccgcgcctactacttcaagtccgagatcacctccgccgagctgaccgccctgttccctgaggccaccaccgccaaccagaaggccctggagtacaccgaggactaccagtccatcgagaagaacgcccagatcacccagggcgacaagtcccgcaaggagctcgggctgggcatcgacctgctgctgaccttcatggaggccgtgaacaagaaggcccgcgtggtgaagaacgaggcccgcttcctgctgatcgccatccagatgaccgccgaggtggcccgcttccgctacatccagaacctggtgaccaagaacttccccaacaagttcgactccgacaacaaggtgatccagttcgaggtcagctggcgcaagatctccaccgccatctacggcgacgccaagaacggcgtgttcaacaaggactacgacttcggcttcggcaaggtgcgccaggtgaaggacctgcagatgggcctgctgatgtacctgggcaagcccaagtag

SEQ ID NO: 24 - DNA, 1233 bp; single-stranded, unknown
  CDS 1..1230: /product= “FGF-SAP”
atggcagcgggttctattactaccctgccggcgctgccggaggacggcggttctggcgctttcccaccgggccactttaaggacccgaaacgcctgtattgcaaaaacggtggttttttcctgcgtatccacccggatggccgcgtcgatggcgtccgcgaaaagtctgatccgcacatcaaactgcaattgcaagcagaggaacgcggtgttgtaagcatcaagggcgtttgcgcgaatcgttacctggcgatgaaagaggatggccgcctgctggcctccaagtgtgtaaccgatgaatgcttcttctttgaacgtctggagtcgaacaattataacacctatcgtagccgtaagtacacctcgtggtacgtagcattgaaacgcaccggtcagtacaaactgggttcgaagacgggtccaggtcagaaagcaattctgttcctgccaatgtcggccaaatcggccatggtcacttctatcacgctggatctggtcaacccgaccgctggtcagtacagctcgtttgtcgataagattcgtaataatgtgaaagatccgaatttaaaatacggtggcacggatattgcagtgattggcccgccgtctaaggaaaagttcttgcgtattaactttcaaagctctcgcggcactgtgtctctgggcttaaaacgcgataatttgtacgttgtagcgtacctggcgatggataataccaatgtaaaccgtgcttactatttcaaaagcgaaattacctctgctgaactgactgcattattcccggaagcgactactgccaatcagaaagccctggaatataccgaagattatcagtcgattgaaaaaaacgcgcaaattacccagggcgacaaatcgcgcaaagagttgggtctgggtattgacctgctgctgacgtttatggaggcggtcaacaaaaaagctcgtgtagtgaaaaacgaagctcgctttctgctgatcgctattcaaatgactgctgaagttgctcgtttccgttacattcagaacttggttactaagaactttccgaacaaattcgactccgataataaggttattcagttcgaagtgagctggcgcaagatttcgacggctatttatggcgatgccaaaaacggcgtatttaacaaagattatgacttcggttttggcaaggttcgtcaggtgaaagatttgcagatgggtctgctgatgtacttgggcaagccgaaataa

SEQ ID NO: 25 - DNA (cDNA), 465 bp; double-stranded, unknown
  CDS 1..465
  mat_peptide 1..465: /product= “bFGF”
atggcagcaggatcaataacaacattacccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagcttcaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcattgaaacgaactgggcagtataaacttggatccaaaacaggacctgggcagaaagctatactttttcttccaatgtctgctaagagc

SEQ ID NO: 26 - DNA (genomic) (A) NAME/KEY Gly4Ser with NcoI ends, 27 bp; single-stranded, linear
  CDS 3..26
ccatgggcggcggcggctctgccatgg

SEQ ID NO: 27 - DNA (genomic) (A) NAME/KEY (Gly4Ser)2 with NcoI ends, 42 bp; single-stranded, linear
  CDS 3..41
ccatgggcggcggcggctctggcggcggcggctctgccatgg

SEQ ID NO: 28 - DNA (genomic) (A) NAME/KEY (Ser4Gly)4 with NcoI ends, 75 bp; single-stranded, linear
  CDS 3..74
ccatggcctcgtcgtcgtcgggctcgtcgtcgtcgggctcgtcgtcgtcgggctcgtcgtcgtcgggcgccatgg

SEQ ID NO: 29 - DNA (genomic) (A) NAME/KEY (Ser4Gly)2, 45 bp; single-stranded, linear
  CDS 3..45
ccatggcctcgtcgtcgtcgggctcgtcgtcgtcgggcgccatgg

SEQ ID NO: 30 - protein, 8 aa
  CDS 1..8: /product= Flexible linker
AAPAAAPA

SEQ ID NO: 31 - protein, 23 aa
  /note= “Endosome-disruptive peptide INF”
GLFEAIEGFIENGWEGMIDGGGC

SEQ ID NO: 32 - protein, 24 aa
  /note= “Endosome-disruptive peptide INF”
GLFEAIEGFIENGWEGMIDGWYGC

SEQ ID NO: 33 - DNA (genomic), 30 bp; single-stranded, linear
  misc_recomb 6..11: /standard_name= “EcoRI Restriction Site”
  sig_peptide 12..30: /function= “N-terminal extension” /product= “Native sapor
ctgcagaattcgcatggatcctgcttcaat

SEQ ID NO: 34 - DNA (genomic), 30 bp; single-stranded, linear
  misc_recomb 6..11: /standard_name= ”EcoRI Restriction Site“
  terminator 23..25: /note= ”Anti-sense stop codon“
  mat_peptide 26..30: /note= ”Anti-sense to carboxyl terminus of mature peptide“
  anti-sense: YES
ctgcagaattcgcctcgtttgactactttg

SEQ ID NO: 35 - DNA (cDNA), 804 bp; double-stranded, unknown
  CDS 1..804
  misc_feature 1..804: /note= ”Nucleotide sequence corresponding to the clone M13 mp18-G1“
  mat_peptide 46..804: /product= ”Saporin“
gcatggatcctgcttcaattttcagcttggacaacaactgatgcggtcacatcaatcacattagatctagtaaatccgaccgcgggtcaatactcatcttttgtggataaaatccgaaacaacgtaaaggatccaaacctgaaatacggtggtaccgacatagccgtgataggcccaccttctaaagaaaaattccttagaattaatttccaaagttcccgaggaacggtctcacttggcctaaaacgcgataacttgtatgtggtcgcgtatcttgcaatggataacacgaatgttaatcgggcatattacttcagatcagaaattacttccgccgagttaaccgcccttttcccagaggccacaactgcaaatcagaaagctttagaatacacagaagattatcagtcgatcgaaaagaatgcccagataacacagggagataaatcaagaaaagaactcgggttggggatcgacttacttttgacgtccatggaagcagtgaacaagaaggcacgtgtggttaaaaacgaagctaggtttctgcttatcgctattcaaatgacagctgaggtagcacgatttcggtacattcaaaacttggtaactaagaacttccccaacaagttcgactcggataacaaggtgattcaatttgaagtcagctggcgtaagatttctacggcaatatacggagatgccaaaaacggcgtgtttaataaagattatgatttcgggtttggaaaagtgaggcaggtgaaggacttgcaaatgggactccttatgtatttgggcaaaccaaag

SEQ ID NO: 36 - DNA (cDNA), 804 bp; double-stranded, unknown
  CDS 1..804
  misc_feature 1..804: /note= ”Nucleotide sequence corresponding to the clone M13 mp18-G2“
  mat_peptide 46..804: /product= ”Saporin“
gcatggatcctgcttcaattttcagcttggacaacaactgatgcggtcacatcaatcacattagatctagtaaatccgactgcgggtcaatactcatcttttgtggataaaatccgaaacaacgtaaaggatccaaacctgaaatacggtggtaccgacatagccgtgataggcccaccttctaaagataaattccttagaattaatttccaaagttcccgaggaacggtctcacttggcctaaaacgcgataacttgtatgtggtcgcgtatcttgcaatggataacacgaatgttaatcgggcatattacttcaaatcagaaattacttccgccgagttaaccgcccttttcccagaggccacaactgcaaatcagaaagctttagaatacacagaagattatcagtcgatcgaaaagaatgcccagataacacagggagataaaagtagaaaagaactcgggttggggatcgacttacttttgacgttcatggaagcagtgaacaagaaggcacgtgtggttaaaaacgaagctaggtttctgcttatcgctattcaaatgacagctgaggtagcacgatttaggtacattcaaaacttggtaactaagaacttccccaacaagttcgactcggataacaaggtgattcaatttgaagtcagctggcgtaagatttctacggcaatatacggggatgccaaaaacggcgtgtttaataaagattatgatttcgggtttggaaaagtgaggcaggtgaaggacttgcaaatgggactccttatgtatttgggcaaaccaaag

SEQ ID NO: 37 - DNA (cDNA), 804 bp; double-stranded, unknown
  CDS 1..804
  misc_feature 1..804: /note= ”Nucleotide sequence corresponding to the clone M13 mp18-G7“
  mat_peptide 46..804: /product= ”Saporin“
gcatggatcctgcttcaattttcagcttggacaacaactgatgcggtcacatcaatcacattagatctagtaaatccgaccgcgggtcaatactcatcttttgtggataaaatccgaaacaacgtaaaggatccaaacctgaaatacggtggtaccgacatagccgtgataggcccaccttctaaagaaaaattccttagaattaatttccaaagttcccgaggaacggtctcacttggcctaaaacgcgataacttgtatgtggtcgcgtatcttgcaatggataacacgaatgttaatcgggcatattacttcagatcagaaattacttccgccgagttaaccgcccttttcccagaggccacaactgcaaatcagaaagctttagaatacacagaagattatcagtcgatcgaaaagaatgcccagataacacagggagataaatcaagaaaagaactcgggttggggatcgacttacttttgacgtccatggaagcagtgaacaagaaggcacgtgtggttaaaaacgaagctagattccttcttatcgctattcagatgacggctgaggcagcacgatttaggtacatacaaaacttggtaatcaagaactttcccaacaagttcaactcggaaaacaaagtgattcagtttgaggttaactggaaaaaaatttctacggcaatatacggggatgccaaaaacggcgtgtttaataaagattatgatttcgggtttggaaaagtgaggcaggtgaaggacttgcaaatgggactccttatgtatttgggcaaaccaaag

SEQ ID NO: 38 - DNA (cDNA), 804 bp; double-stranded, unknown
  CDS 1..804
  misc_feature 1..804: /note= ”Nucleotide sequence corresponding to the clone M13 mp18-G9“
  mat_peptide 46..804: /product= ”Saporin“
gcatggatcctgcttcaattttcagcttggacaacaactgatgcggtcacatcaatcacattagatctagtaaatccgaccgcgggtcaatactcatcttttgtggataaaatccgaaacaacgtaaaggatccaaacctgaaatacggtggtaccgacatagccgtgataggcccaccttctaaagaaaaattccttagaattaatttccaaagttcccgaggaacggtctcacttggcctaaaacgcgataacttgtatgtggtcgcgtatcttgcaatggataacacgaatgttaatcgggcatattacttcagatcagaaattacttccgccgagttaaccgcccttttcccagaggccacaactgcaaatcagaaagctttagaatacacagaagattatcagtcgattgaaaagaatgcccagataacacaaggagatcaaagtagaaaagaactcgggttggggattgacttactttcaacgtccatggaagcagtgaacaagaaggcacgtgtggttaaagacgaagctagattccttcttatcgctattcagatgacggctgaggcagcgcgatttaggtacatacaaaacttggtaatcaagaactttcccaacaagttcaactcggaaaacaaagtgattcagtttgaggttaactggaaaaaaatttctacggcaatatacggggatgccaaaaacggcgtgtttaataaagattatgatttcgggtttggaaaagtgaggcaggtgaaggacttgcaaatgggactccttatgtatttgggcaaaccaaag

SEQ ID NO: 39 - DNA, 30 bp; single-stranded, linear
  /note= ”Primer for SAP-6“
catatgtgtgtcacatcaatcacattagat

SEQ ID NO: 40 - DNA, 21 bp; single-stranded, linear
  /note= ”Primer for SAP-6“
caggtttggatcctttacgtt

SEQ ID NO: 41 - DNA, 66 bp; single-stranded, linear
  /note= ”Primer for Mammalian Codon Preferred Saporin“
cgtatcaggcggccgccgccatggtgacctccatcaccctggacctggtgaaccccaccgccggcc

SEQ ID NO: 42 - DNA, 69 bp; single-stranded, linear
  /note= ”Primer for Mammalian Codon Preferred Saporin“
ttggggtccttcacgttgttgcggatcttgtccacgaaggaggagtactggccggcggtggggttcacc

SEQ ID NO: 43 - DNA, 65 bp; single-stranded, linear
  /note= ”Primer for Mammalian Codon Preferred Saporin“
aacaacgtgaaggaccccaacctgaagtacggcggcaccgacatcgccgtgatcggccccccctc

SEQ ID NO: 44 - DNA, 65 bp; single-stranded, linear
  /note= ”Primer for Mammalian Codon Preferred Saporin“
gtgccgcgggaggactggaagttgatgcgcaggaacttctccttggagggggggccgatcacggc

SEQ ID NO: 45 - DNA, 75 bp; single-stranded, linear
  /note= ”Primer for Mammalian Codon Preferred Saporin“
ctcccgcggcaccgtgtccctgggcctgaagcgcgacaacctgtacgtggtggcctacctggccatggacaacac

SEQ ID NO: 46 - DNA, 77 bp; single-stranded, linear
  /note= ”Primer for Mammalian Codon Preferred Saporin“
gcggtcagctcggcggaggtgatctcggacttgaagtagtaggcgcggttcacgttggtgttgtccatggccaggta

SEQ ID NO: 47 - DNA, 78 bp; single-stranded, linear
  /note= ”Primer for Mammalian Codon Preferred Saporin“
gccgagctgaccgccctgttccctgaggccaccaccgccaaccagaaggccctggagtacaccgaggactaccagtcc

SEQ ID NO: 48 - DNA, 76 bp; single-stranded, linear
  /note= ”Primer for Mammalian Codon Preferred Saporin“
agcccgagctccttgcgggacttgtcgccctgggtgatctgggcgttcttctcgatggactggtagtcctcggtgt

SEQ ID NO: 49 - DNA, 74 bp; single-stranded, linear
  /note= ”Primer for Mammalian Codon Preferred Saporin“
tatagaattcctcgggctgggcatcgacctgctgctgaccttcatggaggccgtgaacaagaaggcccgcgtgg

SEQ ID NO: 50 - DNA, 68 bp; single-stranded, linear
  /note= ”Primer for Mammalian Codon Preferred Saporin“
cggcggtcatctggatggcgatcagcaggaagcgggcctcgttcttcaccacgcgggccttcttgttc

SEQ ID NO: 51 - DNA, 70 bp; single-stranded, linear
  /note= ”Primer for Mammalian Codon Preferred Saporin“
cgccatccagatgaccgccgaggtggcccgcttccgctacatccagaacctggtgaccaagaacttcccc

SEQ ID NO: 52 - DNA, 76 bp; single-stranded, linear
  /note= ”Primer for Mammalian Codon Preferred Saporin“
ggcggatcccagctgacctcgaactggatcaccttgttgtcggagtcgaacttgttggggaagttcttggtcacca

SEQ ID NO: 53 - DNA, 61 bp; single-stranded, linear
  /note= ”Primer for Mammalian Codon Preferred Saporin“
ccgggatccgtcagctggcgcaagatctccaccgccatctacggcgacgccaagaacggcg

SEQ ID NO: 54 - DNA, 64 bp; single-stranded, linear
  /note= ”Primer for Mammalian Codon Preferred Saporin“
gcaccttgccgaagccgaagtcgtagtccttgttgaacacgccgttcttggcgtcgccgtagat

SEQ ID NO: 55 - DNA, 58 bp; single-stranded, linear
  /note= ”Primer for Mammalian Codon Preferred Saporin“
ttcggcttcggcaaggtgcgccaggtgaaggacctgcagatgggcctgctgatgtacc

SEQ ID NO: 56 - DNA, 52 bp; single-stranded, linear
  /note= ”Primer for Mammalian Codon Preferred Saporin“
tgaacgtggcggccgcctacttgggcttgcccaggtacatcagcaggcccat

SEQ ID NO: 57 - DNA (genomic), 17 bp; single-stranded, linear
aggagtgtctgctaacc

SEQ ID NO: 58 - DNA (genomic), 24 bp; single-stranded, linear
ttctaaatcggttaccgatgactg

SEQ ID NO: 59 - DNA, 30 bp; single-stranded, linear
catatgtgtgagctactgtcgccaccgctc

SEQ ID NO: 60 - DNA, 30 bp; single-stranded, linear
ggatccgagcacctggtatatcggtggggg

SEQ ID NO: 61 - DNA (genomic), 59 bp; single-stranded, linear
  /product= bacteriophage lambda CII ribosome binding site
gtcgaccaagcttgggcatacattcaatcaattgttatctaaggaaatacttacatatg

SEQ ID NO: 62 - DNA (genomic), 60 bp; single-stranded, linear
  /product= trp promoter
gaattcccctgttgacaattaatcatcgaactagttaactagtacgcagcttggctgcag

SEQ ID NO: 63 - DNA (genomic), 19 bp; single-stranded, linear
  misc_recomb 11..16: /standard_name= ”Nco I restriction enzyme recognition site“
  mat_peptide 1..10: /product= ”Carboxy terminus of mature FGF protein“
gctaagagcgccatggaga

SEQ ID NO: 64 - DNA (cDNA), 21 bp; double-stranded, unknown
  CDS 1..12: /product= ”Carboxy terminus of wild type FGF“
  misc_recomb 13..18: /standard_name= ”Nco I restriction enzyme recognition site“
gctaagagctgaccatggaga

SEQ ID NO: 65 - DNA, 60 bp; single-stranded, linear
  /note= ”Primer for Protamine“
tacatgccatggccaggtacagatgctgtcgcagccagagccggagcagatattaccgcc

SEQ ID NO: 66 - DNA, 60 bp; single-stranded, linear
  /note= ”Primer for Protamine“
gcagctccgcctccttcgtctgcgacttctttgtctctggcggtaatatctgctccggct

SEQ ID NO: 67 - DNA, 60 bp; single-stranded, linear
  /note= ”Primer for Protamine“
gacgaaggaggcggagctgccagacacggaggagagccatgaggtgctgccgccccaggt

SEQ ID NO: 68 - DNA, 59 bp; single-stranded, linear
  /note= ”Primer for Protamine“
atatatcctaggttagtgtcttctacatctcggtctgtacctggggcggcagcacctca

SEQ ID NO: 69 - DNA (genomic) (A) NAME/KEY Cathepsin B linker, 36 bp; single-stranded, linear
  CDS 3..35
ccatggccctggccctggccctggccctggccatgg

SEQ ID NO: 70 - DNA (genomic) (A) NAME/KEY Cathepsin D linker, 51 bp; single-stranded, linear
  CDS 3..50
ccatgggccgatcgggcttcctgggcttcggcttcctgggcttcgccatgg

SEQ ID NO: 71 - DNA (genomic) (A) NAME/KEY ”Trypsin linker“, 96 bp; single-stranded, linear
  CDS 3..95
ccatgggccgatcgggcggtgggtgcgctggtaatagagtcagaagatcagtcggaagcagcctgtcttgcggtggtctcgacctgcaggccatgg

SEQ ID NO: 72 - DNA (cDNA), 18 bp; double-stranded, unknown
  CDS 1..18: /product=  Thrombin substrate linker
ctggtgccgcgcggcagc

SEQ ID NO: 73 - DNA (cDNA), 15 bp; double-stranded, unknown
  CDS 1..15: /product= Enterokinase substrate linker
gacgacgacgaccca

SEQ ID NO: 74 - DNA (cDNA), 12 bp; double-stranded, unknown
  CDS 1..12: /product= Factor Xa substrate
atcgaaggtcgt

SEQ ID NO: 75 - DNA (cDNA), 1260 bp; double-stranded, unknown
  CDS 1..1260
  mat_peptide 1..465: /product= ”bFGF“
  mat_peptide 466...501: /product= ”Cathepsin B linker“
  mat_peptide 502..1260: /product= ”Saporin“
atggcagcaggatcaataacaacattacccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagcttcaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcattgaaacgaactgggcagtataaacttggatccaaaacaggacctgggcagaaagctatactttttcttccaatgtctgctaagagcgccatggccctggccctggccctggccctggccatggtcacatcaatcacattagatctagtaaatccgaccgcgggtcaatactcatcttttgtggataaaatccgaaacaacgtaaaggatccaaacctgaaatacggtggtaccgacatagccgtgataggcccaccttctaaagaaaaattccttagaattaatttccaaagttcccgaggaacggtctcacttggcctaaaacgcgataacttgtatgtggtcgcgtatcttgcaatggataacacgaatgttaatcgggcatattacttcaaatcagaaattacttccgccgagttaaccgcccttttcccagaggccacaactgcaaatcagaaagctttagaatacacagaagattatcagtcgatcgaaaagaatgcccagataacacagggagataaaagtagaaaagaactcgggttggggatcgacttacttttgacgttcatggaagcagtgaacaagaaggcacgtgtggttaaaaacgaagctaggtttctgcttatcgctattcaaatgacagctgaggtagcacgatttaggtacattcaaaacttggtaactaagaacttccccaacaagttcgactcggataacaaggtgattcaatttgaagtcagctggcgtaagatttctacggcaatatacggggatgccaaaaacggcgtgtttaataaagattatgatttcgggtttggaaaagtgaggcaggtgaaggacttgcaaatgggactccttatgtatttgggcaaaccaaag

SEQ ID NO: 76 - DNA (cDNA), 1275 bp; double-stranded, unknown
  CDS 1..1275
  mat_peptide 1..465: /product= ”bFGF“
  mat_peptide 466...516: /product= ”Cathepsin D linker“
  mat_peptide 517..1275: /product= ”Saporin“
atggcagcaggatcaataacaacattacccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagcttcaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcattgaaacgaactgggcagtataaacttggatccaaaacaggacctgggcagaaagctatactttttcttccaatgtctgctaagagcgccatgggccgatcgggcttcctgggcttcggcttcctgggcttcgccatggtcacatcaatcacattagatctagtaaatccgaccgcgggtcaatactcatcttttgtggataaaatccgaaacaacgtaaaggatccaaacctgaaatacggtggtaccgacatagccgtgataggcccaccttctaaagaaaaattccttagaattaatttccaaagttcccgaggaacggtctcacttggcctaaaacgcgataacttgtatgtggtcgcgtatcttgcaatggataacacgaatgttaatcgggcatattacttcaaatcagaaattacttccgccgagttaaccgcccttttcccagaggccacaactgcaaatcagaaagctttagaatacacagaagattatcagtcgatcgaaaagaatgcccagataacacagggagataaaagtagaaaagaactcgggttggggatcgacttacttttgacgttcatggaagcagtgaacaagaaggcacgtgtggttaaaaacgaagctaggtttctgcttatcgctattcaaatgacagctgaggtagcacgatttaggtacattcaaaacttggtaactaagaacttccccaacaagttcgactcggataacaaggtgattcaatttgaagtcagctggcgtaagatttctacggcaatatacggggatgccaaaaacggcgtgtttaataaagattatgatttcgggtttggaaaagtgaggcaggtgaaggacttgcaaatgggactccttatgtatttgggcaaaccaaag

SEQ ID NO: 77 - DNA (cDNA), 1251 bp; double-stranded, unknown
  CDS 1..1251
  mat_peptide 1..465: /product= ”bFGF“
  mat_peptide 466..492: /product= ” Gly4Ser linker“
  mat_peptide 493..1251: /product= ”Saporin“
atggcagcaggatcaataacaacattacccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagcttcaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcattgaaacgaactgggcagtataaacttggatccaaaacaggacctgggcagaaagctatactttttcttccaatgtctgctaagagcgccatgggcggcggcggctctgccatggtcacatcaatcacattagatctagtaaatccgaccgcgggtcaatactcatcttttgtggataaaatccgaaacaacgtaaaggatccaaacctgaaatacggtggtaccgacatagccgtgataggcccaccttctaaagaaaaattccttagaattaatttccaaagttcccgaggaacggtctcacttggcctaaaacgcgataacttgtatgtggtcgcgtatcttgcaatggataacacgaatgttaatcgggcatattacttcaaatcagaaattacttccgccgagttaaccgcccttttcccagaggccacaactgcaaatcagaaagctttagaatacacagaagattatcagtcgatcgaaaagaatgcccagataacacagggagataaaagtagaaaagaactcgggttggggatcgacttacttttgacgttcatggaagcagtgaacaagaaggcacgtgtggttaaaaacgaagctaggtttctgcttatcgctattcaaatgacagctgaggtagcacgatttaggtacattcaaaacttggtaactaagaacttccccaacaagttcgactcggataacaaggtgattcaatttgaagtcagctggcgtaagatttctacggcaatatacggggatgccaaaaacggcgtgtttaataaagattatgatttcgggtttggaaaagtgaggcaggtgaaggacttgcaaatgggactccttatgtatttgggcaaaccaaag

SEQ ID NO: 78 - DNA (cDNA), 1266 bp; double-stranded, unknown
  CDS 1..1266
  mat_peptide 1..465: /product= ”bFGF“
  mat_peptide 466..507: /product= ” (Gly4Ser)2 linker“
  mat_peptide 508..1266: /product= ”Saporin“
atggcagcaggatcaataacaacattacccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagcttcaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcattgaaacgaactgggcagtataaacttggatccaaaacaggacctgggcagaaagctatactttttcttccaatgtctgctaagagcgccatgggcggcggcggctctggcggcggcggctctgccatggtcacatcaatcacattagatctagtaaatccgaccgcgggtcaatactcatcttttgtggataaaatccgaaacaacgtaaaggatccaaacctgaaatacggtggtaccgacatagccgtgataggcccaccttctaaagaaaaattccttagaattaatttccaaagttcccgaggaacggtctcacttggcctaaaacgcgataacttgtatgtggtcgcgtatcttgcaatggataacacgaatgttaatcgggcatattacttcaaatcagaaattacttccgccgagttaaccgcccttttcccagaggccacaactgcaaatcagaaagctttagaatacacagaagattatcagtcgatcgaaaagaatgcccagataacacagggagataaaagtagaaaagaactcgggttggggatcgacttacttttgacgttcatggaagcagtgaacaagaaggcacgtgtggttaaaaacgaagctaggtttctgcttatcgctattcaaatgacagctgaggtagcacgatttaggtacattcaaaacttggtaactaagaacttccccaacaagttcgactcggataacaaggtgattcaatttgaagtcagctggcgtaagatttctacggcaatatacggggatgccaaaaacggcgtgtttaataaagattatgatttcgggtttggaaaagtgaggcaggtgaaggacttgcaaatgggactccttatgtatttgggcaaaccaaag

SEQ ID NO: 79 - DNA (cDNA), 1320 bp; double-stranded, unknown
  CDS 1..1320
  mat_peptide 1..465: /product= ”bFGF“
  mat_peptide 466..561: /product= ”Trypsin linker“
  mat_peptide 562..1320: /product= ”Saporin“
atggcagcaggatcaataacaacattacccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagcttcaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcattgaaacgaactgggcagtataaacttggatccaaaacaggacctgggcagaaagctatactttttcttccaatgtctgctaagagcgccatgggccgatcgggcggtgggtgcgctggtaatagagtcagaagatcagtcggaagcagcctgtcttgcggtggtctcgacctgcaggccatggtcacatcaatcacattagatctagtaaatccgaccgcgggtcaatactcatcttttgtggataaaatccgaaacaacgtaaaggatccaaacctgaaatacggtggtaccgacatagccgtgataggcccaccttctaaagaaaaattccttagaattaatttccaaagttcccgaggaacggtctcacttggcctaaaacgcgataacttgtatgtggtcgcgtatcttgcaatggataacacgaatgttaatcgggcatattacttcaaatcagaaattacttccgccgagttaaccgcccttttcccagaggccacaactgcaaatcagaaagctttagaatacacagaagattatcagtcgatcgaaaagaatgcccagataacacagggagataaaagtagaaaagaactcgggttggggatcgacttacttttgacgttcatggaagcagtgaacaagaaggcacgtgtggttaaaaacgaagctaggtttctgcttatcgctattcaaatgacagctgaggtagcacgatttaggtacattcaaaacttggtaactaagaacttccccaacaagttcgactcggataacaaggtgattcaatttgaagtcagctggcgtaagatttctacggcaatatacggggatgccaaaaacggcgtgtttaataaagattatgatttcgggtttggaaaagtgaggcaggtgaaggacttgcaaatgggactccttatgtatttgggcaaaccaaag

SEQ ID NO: 80 - DNA (cDNA), 1299 bp; double-stranded, unknown
  CDS 1..1299
  mat_peptide 1..465: /product= ”bFGF“
  mat_peptide 466..540: /product= ”(Ser4Gly)4linker“
  mat_peptide 541..1299: /product= ”Saporin“
atggcagcaggatcaataacaacattacccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagcttcaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcattgaaacgaactgggcagtataaacttggatccaaaacaggacctgggcagaaagctatactttttcttccaatgtctgctaagagcgccatggcctcgtcgtcgtcgggctcgtcgtcgtcgggctcgtcgtcgtcgggctcgtcgtcgtcgggcgccatggtcacatcaatcacattagatctagtaaatccgaccgcgggtcaatactcatcttttgtggataaaatccgaaacaacgtaaaggatccaaacctgaaatacggtggtaccgacatagccgtgataggcccaccttctaaagaaaaattccttagaattaatttccaaagttcccgaggaacggtctcacttggcctaaaacgcgataacttgtatgtggtcgcgtatcttgcaatggataacacgaatgttaatcgggcatattacttcaaatcagaaattacttccgccgagttaaccgcccttttcccagaggccacaactgcaaatcagaaagctttagaatacacagaagattatcagtcgatcgaaaagaatgcccagataacacagggagataaaagtagaaaagaactcgggttggggatcgacttacttttgacgttcatggaagcagtgaacaagaaggcacgtgtggttaaaaacgaagctaggtttctgcttatcgctattcaaatgacagctgaggtagcacgatttaggtacattcaaaacttggtaactaagaacttccccaacaagttcgactcggataacaaggtgattcaatttgaagtcagctggcgtaagatttctacggcaatatacggggatgccaaaaacggcgtgtttaataaagattatgatttcgggtttggaaaagtgaggcaggtgaaggacttgcaaatgggactccttatgtatttgggcaaaccaaag

SEQ ID NO: 81 - DNA (cDNA), 1269 bp; double-stranded, unknown
  CDS 1..1269
  mat_peptide 1..465: /product= ”bFGF“
  mat_peptide 466..510: /product= ”(Ser4Gly)2 linker“
  mat_peptide 511..1269: /product= ”Saporin“
atggcagcaggatcaataacaacattacccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagcttcaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcattgaaacgaactgggcagtataaacttggatccaaaacaggacctgggcagaaagctatactttttcttccaatgtctgctaagagcgccatggcctcgtcgtcgtcgggctcgtcgtcgtcgggcgccatggtcacatcaatcacattagatctagtaaatccgaccgcgggtcaatactcatcttttgtggataaaatccgaaacaacgtaaaggatccaaacctgaaatacggtggtaccgacatagccgtgataggcccaccttctaaagaaaaattccttagaattaatttccaaagttcccgaggaacggtctcacttggcctaaaacgcgataacttgtatgtggtcgcgtatcttgcaatggataacacgaatgttaatcgggcatattacttcaaatcagaaattacttccgccgagttaaccgcccttttcccagaggccacaactgcaaatcagaaagctttagaatacacagaagattatcagtcgatcgaaaagaatgcccagataacacagggagataaaagtagaaaagaactcgggttggggatcgacttacttttgacgttcatggaagcagtgaacaagaaggcacgtgtggttaaaaacgaagctaggtttctgcttatcgctattcaaatgacagctgaggtagcacgatttaggtacattcaaaacttggtaactaagaacttccccaacaagttcgactcggataacaaggtgattcaatttgaagtcagctggcgtaagatttctacggcaatatacggggatgccaaaaacggcgtgtttaataaagattatgatttcgggtttggaaaagtgaggcaggtgaaggacttgcaaatgggactccttatgtatttgggcaaaccaaag

SEQ ID NO: 82 - DNA, 36 bp; single-stranded, linear
tatatagaattcgtagacaaagctaatgcaccaaaa

SEQ ID NO: 83 - DNA, 36 bp; single-stranded, linear
tatatactcgagcactgggtggtgttcagggaagct

SEQ ID NO: 84 - DNA, 700 bp; single-stranded, linear
gaattcgtagacaaagctaatgcaccaaaaaaatgaatgtagttatagtaatgctaacatccaaattcctctttgtaagacataggcctgtcaaccttgtctccatacttcaattcctatttccactcacctccctcaagaacttgatttataaacagtgtgcctaccataaaatcatcactccctctatgtatttatagacgactgaaggaatatctttcttctttgcatgctaccgtggtagaagggttttaaaagtccgtgctaggcagaggcagccctttctgcccctttctgttctcagtttattaggaaatggcctgaaattccagcatgatagcaagctggcatcctctgtggaatgtgcaaaccatgcctgcatctgcccattaccctagctcagtgtctctgggcatttctgcagttgttctgaaggcttggcgtgtttatctcccacaggcggctgaaccgcctcccgtttcatgagcagaccagtggaatgcagtggaagagacccaggcctccggccacccagattagagagttttgtgctgaggtccctatatggttgtgttagactgaacgacaggctcaagtctgtctttgctccttgtttgggaagcaagtgggaggagagcaggccaaggggctatataacccttcagctttcagcttccctgaacaccacccagtgctcgag

SEQ ID NO: 85 - DNA, 80 bp; single-stranded, linear
tatagaattcctgtgtctaacgggggtgtgtgctctccctcctctggcgaccatgaggaaacccccggcaggacaaggtg

SEQ ID NO: 86 - DNA, 76 bp; single-stranded, linear
cctgcccagtgactggcagatgagaagctccattgtcgccccagggagtatggggcacaggcgcctccttgggttg

SEQ ID NO: 87 - DNA, 76 bp; single-stranded, linear
atctgccagtcactgggcaggggctacgtgccagggaccatgctagttctctgcacaccttgtcctgccgggggtt

SEQ ID NO: 88 - DNA, 81 bp; single-stranded, linear
tataggatcctggactcagctgaggcccgcctgggcaccctggggctcccgggaggcagacaacccaaggaggcgcctgtg

SEQ ID NO: 89 - DNA, 73 bp; single-stranded, linear
tataggatccgggcctcagctgagtccaggcctcggggacagtccgtgcacgctcctggggctgggggcgggc

SEQ ID NO: 90 - DNA, 81 bp; single-stranded, linear
ttcatgagctcacgcctttccagagaaatcccttaatgccgccattctgctggtggcatatatagggagggctcggccttg

SEQ ID NO: 91 - DNA, 81 bp; single-stranded, linear
ggaaaggcgtgagctcatgaagaaggctgctcagtcagcagaaacgtggctgggacaagtgcccgcccccagccccaggag

SEQ ID NO: 92 - DNA, 88 bp; single-stranded, linear
tatatactcgagcggggacctggaggctggcaggagtcagcggggcctctggcagccagtgtggagccaaggccgagccctccctata

SEQ ID NO: 93 - DNA, 462 bp; single-stranded, linear
gaattcctgtgtctaacgggggtgtgtgctctccctcctctggcgaccatgaggaaacccccggcaggacaaggtgtgcagagaactagcatggtccctggcacgtagcccctgcccagtgactggcagatgagaagctccattgtcgccccagggagtatggggcacaggcgcctccttgggttgtctgcctcccgggagccccagggtgcccaggcgggcctcagctgagtccaggcctcggggacagtccgtgcacgctcctggggctgggggcgggcacttgtcccagccacgtttctgctgactgagcagccttcttcatgagctcacgcctttccagagaaatcccttaatgccgccattctgctggtggcatatatagggagggctcggccttggctccacactggctgccagaggccccgctgactcctgccagcctccaggtccccgctcgag

SEQ ID NO: 94 - DNA, 35 bp; single-stranded, linear
attaattatagatctcagctcttagcagacattgg

SEQ ID NO: 95 - DNA, 30 bp; single-stranded, linear
gcttgggcatacattcaatcaattgttatc

SEQ ID NO: 96 - DNA, 40 bp; single-stranded, linear
cgtaatatggtctcaatatgtaagtattgtagttattaga

SEQ ID NO: 97 - DNA, 39 bp; single-stranded, linear
cgtaatatggtctcaatatcaaggaaatacaccagttgg

SEQ ID NO: 98 - DNA, 35 bp; single-stranded, linear
cggatatggtctcagaatacaccagttggtatgtg

SEQ ID NO: 99 - DNA, 38 bp; single-stranded, linear
cgtaatatggtctcaattcccttgaccggtaagtattg

SEQ ID NO: 100 - DNA, 35 bp; single-stranded, linear
cgaatatggtctcagcaaccagttggtatgtggca

SEQ ID NO: 101 - DNA, 37 bp; single-stranded, linear
cgtaacatggtctcattgctttccttgaccggtaagt

SEQ ID NO: 102 - DNA, 37 bp; single-stranded, linear
gctattaggtctcagcatatgtggcattgaaacgaac

SEQ ID NO: 103 - DNA, 38 bp; single-stranded, linear
cgaattaggtctcaatgcactggtgtatttccttgacc